(19) United States
(12) Patent Application Publication
(10) Pub. No.: US 2004/0214832 A1
(43) Pub. Date: Oct. 28, 2004

(54) Piperazine derivative renin inhibitors

(76) Inventors: Cuiman Cai, Ann Arbor, MI (US); Emma Hazel Clay, Lowestoft (GB); Dennis M. Downing, Ann Arbor, MI (US); Jeremy John Edmunds, Ypsilanti, MI (US); Daniel Dale Holsworth, Ann Arbor, MI (US); Tingsheng Li, Edmonton (CA); Noel Aaron Powell, Dexter, MI (US)

Correspondence Address: WARNER-LAMBERT COMPANY, 2800 PLYMOUTH RD, ANN ARBOR, MI, 48105, US

(21) Appl. No.: 10/811,134

(22) Filed: Mar. 26, 2004

Related U.S. Application Data
(60) Provisional application No. 60/461,931, filed on Apr. 10, 2003.
(60) Provisional application No. 60/542,306, filed on Feb. 9, 2004.

(51) Int. Cl. A61K 31/495

(52) U.S. Cl. 514/255.02; 514/255.03; 544/384; 544/392

(57) ABSTRACT

Disclosed are piperazine derivatives, their manufacture and use as inhibitors of renin.

The present application claims priority under 35 USC section 119(e) to U.S. Provisional Application Serial No. 60/461,931, filed Apr. 10, 2003 and to U.S. Provisional Application Serial No. 60/542,306, filed Feb. 9, 2004.

### FIELD OF THE INVENTION

This invention relates to piperazine derivative useful as inhibitors of renin.

### BACKGROUND OF THE INVENTION

Renin is an endopeptidase (molecular weight about 40,000) produced and secreted by the juxtaglomerular cells of the kidney, which cleaves the naturally-occurring plasma glycoprotein, antiotensinogen. Renin cleaves angiotensinogen, its protein substrate, to split off the hemodynamically-inactive N-terminal decapeptide, angiotensin I, which is converted in the lungs, kidney or other tissue by angiotensin-converting enzyme to the potent pressor octapeptide, angiotensin II. Angiotensin II is known to be a potent pressor substance, i.e., a substance that is capable of inducing a significant increase in blood pressure, and is believed to act by causing the constriction of blood vessels and the release of the sodium-retaining hormone aldosterone from the adrenal gland. Thus, the renin-angiotensinogen system has been implicated as a causative factor in certain forms of hypertension and congestive heart failure.

Inhibitors of angiotensin I converting enzyme have proven useful in the modulation of the renin-angiotensin system. Consequently, specific inhibitors of the limiting enzymatic step that ultimately regulates angiotensin II production, the action of renin on its substrate, are sought as effective therapeutic agents in the treatment of hypertension, and congestive heart failure.

### SUMMARY OF THE INVENTION

Generally, the present invention relates to piperazine and piperazinone derivative rennin inhibitors. One embodiment is a compound of Formula I

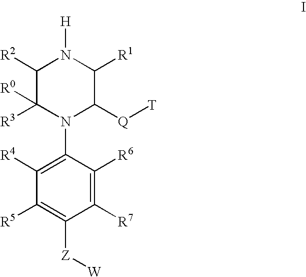

or a pharmaceutically acceptable salt thereof, where

R¹ and R² are independently hydrogen or unsubstituted C₁-C₃ alkyl;

R³ is hydrogen, oxo, or thioxo;

R⁰ is hydrogen or unsubstituted C₁-C₃ alkyl provided that when R³ is oxo or thioxo R⁰ is absent;

R⁴, R⁵, R⁶, and R⁷ are independently hydrogen, halogen, carboxyl, substituted or unsubstituted C₁-C₃ alkoxy, or substituted or unsubstituted C₁-C₃ alkyl;

Q is —(CH₂)₁₋₆—C(O)—O—(CH₂)₀₋₆—, —(CH₂)₁₋₆—O—C(O)—(CH₂)₀₋₆—, —(CH₂)₁₋₆—C(O)—NR⁸—(CH₂)₀₋₆—, —(CH₂)₁₋₆—NR⁹—C(O)—(CH₂)₀₋₆—, —(CH₂)₁₋₆—NR¹⁰—S(O)₂—(CH₂)₀₋₆—, —(CH₂)₁₋₆—S(O)₂—NR¹⁰—(CH₂)₀₋₆—, —(CH₂)₁₋₆—NR¹¹—C(O)—NR¹²—(CH₂)₀₋₆—, or —CH₂—(C₁-C₆ alkylene) where 1 to 3 nonadjacent methylene units of the alkylene group are replaced with O, NR¹³, S or a combination thereof;

T is substituted or unsubstituted aryl, substituted or unsubstituted heteroaryl, or substituted or unsubstituted C₁-C₁₂ alkyl;

W is absent, substituted or unsubstituted aryl, or substituted or unsubstituted heteroaryl;

Z is —(CH₂)₀₋₆-cycloalkylene-(CH₂)₀₋₆— where 0 to 6 nonadjacent methylene units are replaced with O, NR¹⁶, S or a combination thereof,

—(CH₂)₀₋₆-heterocycloalkylene-(CH₂)₀₋₆— where 0 to 6 nonadjacent methylene units are replaced with O, NR¹⁶, S or a combination thereof,

—(CH₂)₀₋₆-arylene-(CH₂)₀₋₆— where 0 to 6 nonadjacent methylene units are replaced with O, NR¹⁶, S or a combination thereof,

—(CH₂)₀₋₆-heteroarylene-(CH₂)₀₋₆— where 0 to 6 nonadjacent methylene units are replaced with O, NR¹⁶, S or a combination thereof,

—(CH₂)₀₋₆—C(O)—NR¹⁶—(CH₂)₀₋₆— where 0 to 6 nonadjacent methylene units are replaced with O, NR¹⁶, S or a combination thereof,

—(CH₂)₀₋₆—NR¹⁶—C(O)—(CH₂)₀₋₆— where 0 to 6 nonadjacent methylene units are replaced with O, NR¹⁶, S or a combination thereof,

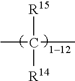

where 1 to 6 nonadjacent

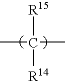

units are replaced with O, NR¹⁶,

S or a combination thereof, or

Z, when W is absent, is hydroxyl, substituted or unsubstituted C₁-C₁₂ alkyl where 1 to 6 nonadjacent methylene units are replaced with O,

NR¹⁶, S or a combination thereof, or —(CH₂)₀₋₆—C(O)—NR¹⁶—(CH₂)₀₋₅—CH₃ where 0 to 6 nonadjacent methylene units are replaced with O,

NR¹⁶, S or a combination thereof;

R⁸, R⁹, R¹⁰, R¹¹, and R¹² are independently hydrogen, substituted or unsubstituted C₁-C₃ alkoxy, or substituted or unsubstituted C₁-C₃ alkyl;

R¹³ and R¹⁶ are independently substituted or unsubstituted C₁-C₃ alkyl or hydrogen; and

R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl where 1 to 6 nonadjacent methylene units are replaced with O, or R¹⁴ and R¹⁵ together with the carbon to which they are attached form a 3- to 6-membered cycloalkylene or heterocycloalkylene ring.

Another embodiment is a pharmaceutical composition comprising a compound of Formula I admixed with a pharmaceutically acceptable carrier, diluent, or excipient.

Another embodiment is a method of inhibiting renin in a mammal comprising administering to the mammal in need thereof an effective amount of a compound of Formula I.

Other embodiments include methods of treating or preventing hypertension, congestive heart failure, stroke, myocardial infarction, glaucoma, or hyperaldosteronism in a mammal comprising administering to the mammal in need thereof an effective amount of a compound of Formula I.

Another embodiment is a method of providing end organ protection in a mammal comprising administering to the mammal in need thereof an effective amount of a compound of Formula I.

Yet another embodiment is a process for preparing a compound of claim 1 including the steps of:

a) acylation of a protected para-hydroxy aniline 1, where P¹ is an amine protecting group, to afford the intermediate 2 where R²⁰ is halo and R² is as defined in claim 1;

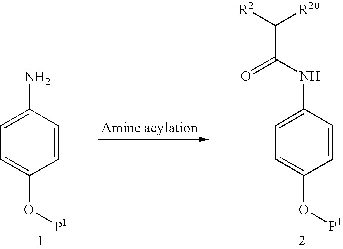

b) contacting 2 with a suitable amine to afford the intermediate 3, where P² is a suitable anime protecting group;

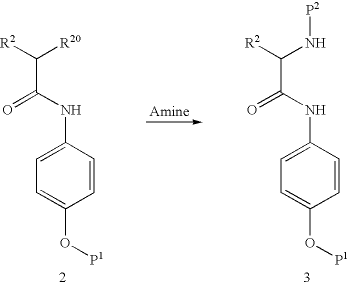

c) contacting 3 with a suitable epoxide to afford the intermediate 4, where R²¹ is halo and R¹ is as defined in claim 1;

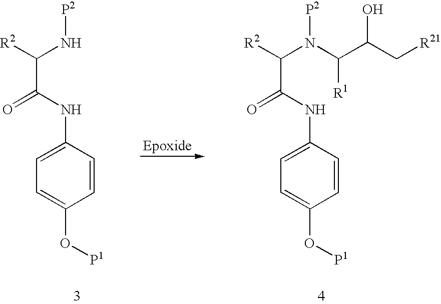

d) cyclization of 4 to afford 5;

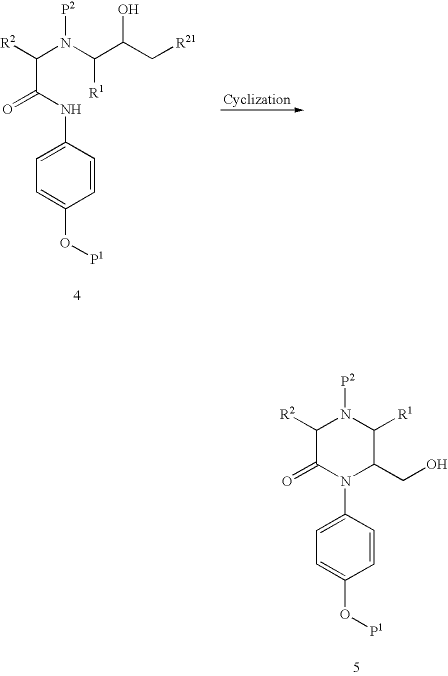

e) deprotection of 5 followed by protection of the piperazinone nitrogen with a suitable amine protecting group, P³, to afford 6;

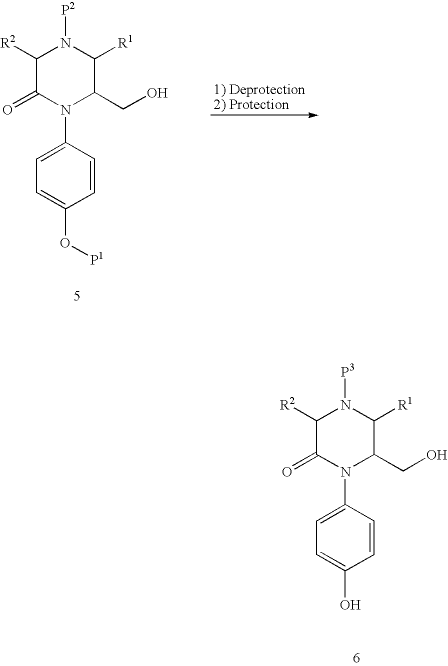

a suitable alkylating agent to afford 7 where R²², along with the oxygen at the 4-position of the phenyl ring, is equivalent to -Z-W as is defined above in Formula I;

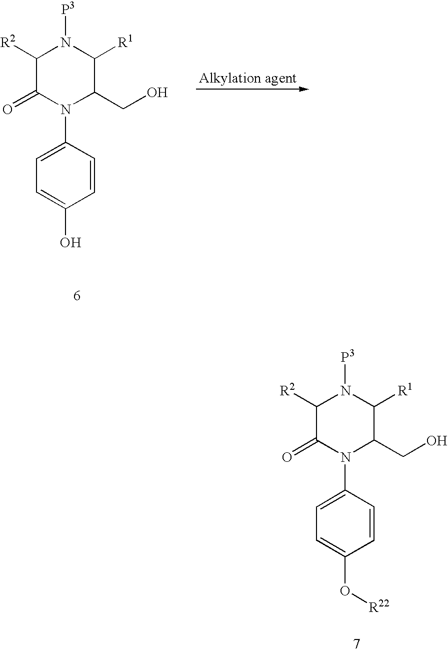

g) contacting 7 with an appropriate alcohol to afford 8, where R²³, along with the hydroxymethyl substituent of the piperazinone, is equivalent to -Q-T as is defined above in Formula I;

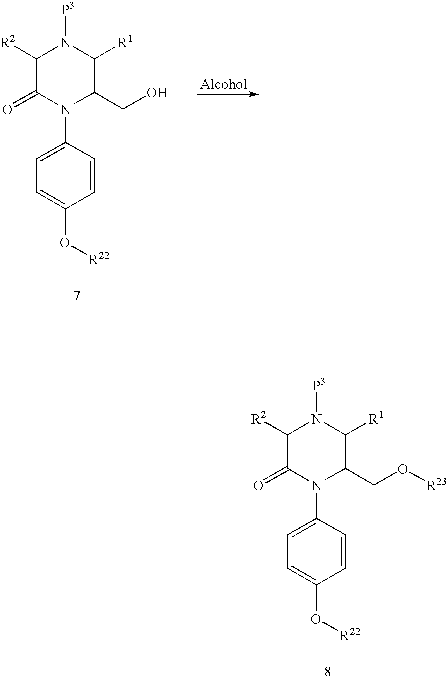

h) deprotection of 8 to afford 9

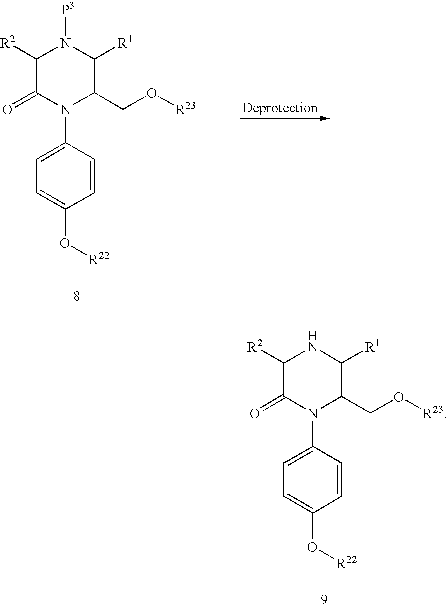

The above summary of the present invention is not intended to describe each disclosed embodiment or every implementation of the present invention. The detailed description which follows more particularly exemplifies these embodiments.

### DETAILED DESCRIPTION OF THE INVENTION

The present invention is believed to be applicable to inhibitors of renin. In particular, the present invention is directed to piperazine and piperazinone derivatives useful as inhibitors of renin. While the present invention is not so limited, an appreciation of various aspects of the invention will be gained through the following discussion and the examples provided below.

Alkyl, alkoxy, etc. denote both straight and branched groups; but reference to an individual radical such as “propyl” embraces only the straight chain radical, a branched chain isomer such as “isopropyl” being specifically referred to.

As used in this specification and the appended claims, the singular forms “a”, “an”, and “the” include plural referents unless the content clearly dictates otherwise. Thus, for example, reference to a composition containing “a compound” includes a mixture of two or more compounds.

The recitation of numerical ranges by endpoints includes all numbers subsumed within that range (e.g. 1 to 5 includes 1, 1.5, 2, 2.75, 3, 3.80, 4, and 5).

The term “halogen” or “halo” as used herein includes chlorine, fluorine, bromine, and iodine.

The term “oxo” as used herein refers to ═O.

The term “thioxo” as used herein refers to ═S.

The term “carboxyl” as used herein refers to

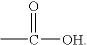

The term “hydroxy” or “hydroxyl” as used herein refers to —OH.

The term “methylene” as used herein refers to —CH₂—.

The term “alkyl” as used herein refers to a monovalent straight or branched hydrocarbon radical having 1 to 12 carbon atoms. Alkyl groups can be unsubstituted or substituted with one or more of the substituents selected from halogen, —OH, —NH₂, or —NH R′, where R′ is unsubstituted C₁-C₃ alkyl. Alkyl groups are assumed to be unsubstituted unless specifically denoted as substituted. Examples of alkyl groups include, but are not limited to, methyl, ethyl, n-propyl, isopropyl, n-butyl, sec-butyl, isobutyl, tert-butyl, n-pentyl, and n-hexyl. Examples of substituted alkyl groups include, but are not limited to, trifluoromethyl, hydroxymethyl, aminomethyl, and methylaminomethyl.

The term “lower” as used herein refers to a group having 1 to 3 carbon atoms. For example “lower alkyl” as used herein refers to a subset of alkyl which means a straight or branched hydrocarbon radical having from 1 to 3 carbon atoms and includes, for example, methyl, ethyl, n-propyl, and isopropyl.

The term “alkylene” as used herein refers to a divalent straight or branched chain hydrocarbon radical having 1 to 12 carbon atoms. Alkylene groups can be unsubstituted or substituted with one or more of the substituents selected from halogen, —OH, —NH₂, or —NH R″, where R″ is unsubstituted C₁-C₃ alkyl. Examples of “alkylene” as used herein include, but are not limited to, methylene, ethylene, propane-1,3-diyl, propane-1,2-diyl, butane-1,4-diyl, pentane-1,5-diyl, and hexane-1,6-diyl.

As used herein, “cycloalkyl” refers to an alicyclic hydrocarbon group having 3 to 8 carbon atoms. Examples of “cycloalkyl” as used herein include, but are not limited to, cyclopropyl, cyclobutyl, cyclopentyl, cyclohexyl, cycloheptyl, and cyclooctyl.

The term “cycloalkylene” as used herein refers to an alicyclic divalent hydrocarbon radical having 3 to 6 carbon atoms. Examples of “cycloalkylene” as used herein include, but are not limited to, cyclopropane-1,1-diyl, cyclopropane-1,2-diyl, cyclobutane-1,2-diyl, cyclopentane-1,1-diyl, cyclopentane-1,3-diyl, cyclohexane-1,1-diyl, cyclohexane-1,2-diyl, cyclohexane-1,3-diyl, cyclohexane-1,4-diyl, cycloheptane-1,4-diyl, and cyclooctane-1,5-diyl.

The term “heterocycloalkyl” as used herein refers to an alicyclic hydrocarbon group having 3 to 6 carbon atoms and containing one to three nonadjacent heteroatomic substitutions independently selected from S, O, and NH. Examples of “heterocycloalkyl” as used herein include, but are not limited to, tetrahydrofuryl, 1,4-dioxyl, 1,3-dioxyl, piperidinyl, pyrrolidinyl, morpholinyl, thiomorpholinyl, tetrahydropyranyl, tetrahydrothiopyranyl, tetrahydrothiophenyl, oxazolidinyl, Isoxazolidinyl, isothiazolidinyl, thiazolidinyl, [1,2]oxathiolanyl, [1,3]oxathiolanyl, [1,2]oxazinanyl, [1,3]oxazinanyl, [1,2]oxathianyl, [1,3]oxathianyl, and [1,4]oxathianyl. For heterocycles containing sulfur, the oxidized sulfur heterocycles containing SO or SO₂ groups are also included. Examples include the sulfoxide and sulfone forms of tetrahydrothiophene.

The term “heterocycloalkylene” as used herein refers to an alicyclic divalent hydrocarbon radical having 3 to 6 carbon atoms and containing one to three nonadjacent heteroatomic substitutions independently selected from S, O, and NH. Examples of “heterocycloalkylene” as used herein include, but are not limited to, tetrahydropyran-4,4-diyl, tetrahydropyran-2,3-diyl, tetrahydropyran-3,4-diyl, tetrahydropyran-2,6-diyl, tetrahydropyran-3,5-diyl, piperidine-4,4-diyl, piperidine-2,3-diyl, piperidine-3,4-diyl, piperidine-2,6-diyl, piperidine-3,5-diyl, tetrahydrothiopyran-4,4-diyl, tetrahydrothiopyran-2,3-diyl, tetrahydrothiopyran-3,4-diyl, tetrahydrothiopyran-2,6-diyl, tetrahydrothiopyran-3,5-diyl, tetrahydrofuran-3,3-diyl, tetrahydrofuran-2,3-diyl, tetrahydrofuran-3,4-diyl, tetrahydrofuran-2,5-diyl, pyrrolidine-3,3-diyl, pyrrolidine-2,3-diyl, pyrrolidine-3,4-diyl, pyrrolidine-2,5-diyl, tetrahydrothiophene-3,3-diyl, tetrahydrothiophene-2,3-diyl, tetrahydrothiophene-3,4-diyl, tetrahydrothiophene-2,5-diyl, morpholine-2,3-diyl, thiomorpholine-2,3-diyl, [1,4]oxathiane-2,3-diyl, oxazolidine-4,5-diyl, [1,3]oxathiolane-4,5-diyl, and thiazolidine-4,5-diyl.

The term “aryl” as used herein means monovalent unsaturated aromatic carbocyclic radicals having a single ring, such as phenyl, or multiple condensed rings, such as naphthyl or anthryl. Aryl groups may be unsubstituted or substituted with 1 to 5 substituents selected from —O(CH₂)₁₋₃CF₃, —NH₂, —OCF₃, —CO₂H, —SO₂(C₁-C₆alkyl), —SO₂NH₂, —SO₂NHR′ and —SO₂NR′R″, where R′ and R″ are as defined above, C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl.

Such an aryl ring may be optionally fused to one or more of another heterocycloalkyl ring(s), heteroaryl ring(s), or cycloalkyl rings. Examples of aryl groups include, but are not limited to, anthryl, naphthyl, phenyl, biphenyl, chromanyl, 2-oxo-4a,8a-dihydro-2H-chromenyl 1,2,3,4-tetrahydroquinolinyl, 2-oxo-1,2,3,4-tetrahydroquinolinyl, 3,4-dihydro-2H-benzo[1,4]oxazinyl, 3-oxo-3,4-dihydro-2H-benzo[1,4]oxazinyl, indanyl, 2,3-dihydroindolyl, 1,2,3,4-tetrahydroquinazolinyl, 2-oxo-1,2,3,4-tetrahydroquinazolinyl, 2,3-dihydrobenzoxazolyl, 1,2,3,4-tetrahydronaphthyl, 1,2,3,4-tetrahydroisoquinolinyl, 1,2,3,4-tetrahydroquinoxalinyl, 1,2,3,4-tetrahydro-cinnolinyl, 1,2,3,4-tetrahydro-phthalazinyl, 2,3-dihydroindolyl, 1,2,3,4-tetrahydroindolyl, Specific examples of those aryl groups disclosed immediately above include 2-oxo-1,2,3,4-tetrahydroquinolin-7-yl, 2-oxo-1,2,3,4-tetrahydroquinolin-6-yl, 4-oxo-1,2,3,4-tetrahydroquinolin-7-yl, 4-oxo-1,2,3,4-tetrahydroquinolin-6-yl, 3-oxo-3,4-dihydro-2H-benzo[1,4]oxazin-6-yl, 3-oxo-3,4-dihydro-2H-benzo[1,4]oxazin-7-yl, indan-6-yl, 2-oxo-1,2,3,4-tetrahydroquinazolin-7-yl, 2,3-dihydrobenzoxazol-5-yl, 2-oxo-4a,8a-dihydro-2H-chromen-7-yl, 2,3-dihydroindol-6-yl, 2-oxo-2,3-dihydroindol-6-yl, and 2,3-dihydro-isoindolyl. Examples of substituted 1,2,3,4-tetrahydroquinolinyl include, but are not limited to, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl and 1-(2-acetoxyethyl)-3,4-dihydro-2H-quinolin-7-yl.

Examples of substituted 3,4-dihydro-2H-benzo[1,4]oxazinyl include, but are not limited to, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, and 4-(2-methoxy-2-oxoethyl)-3-oxo-3,4-dihydro-2H-benzo[1,4]oxazin-6-yl.

Examples of substituted naphthyl include, but are not limited to, 6-methoxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxyethyl)-2-naphthyl, and 7-(2-acetoxyethyl)-2-naphthyl.

Examples of substituted phenyl include, but are not limited to, 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, and 4-acetylaminophenyl.

The term “arylene” as used herein refers to divalent unsaturated aromatic carbocyclic radicals having a single ring, such as phenylene, or multiple condensed rings, such as naphthylene or anthrylene. Arylene groups may be unsubstituted or substituted with those substituents enumerated for aryl. Examples of aryl groups include, but are not limited to, phenylene-1,2-diyl, phenylene-1,3-diyl, phenylene-1,4-diyl, naphthalene-2,7-diyl, naphthalene-2,6-diyl, anthracene-1,4-diyl, anthracene-2,6-diyl, and anthracene-2,7-diyl. Examples of substituted arylene groups include, but are not limited to, 2-fluoro-phenylene-1,3-diyl, 2-fluoro-phenylene-1,4-diyl, 2-chloro-phenylene-1,3-diyl, 2-chloro-phenylene-1,4-diyl, 2-methyl-phenylene-1,3-diyl, 2-methyl-phenylene-1,4-diyl, 2-trifluoromethyl-phenylene-1,3-diyl, and 2-trifluoromethyl-phenylene-1,4-diyl.

The term “heteroaryl” as used herein refers to monovalent aromatic cyclic or polycyclic ring systems having from 1 to 4 nonadjacent heteroatoms independently selected from N, O, and S. Heteroaryl groups may be unsubstituted or substituted with one or more groups enumerated for aryl. Examples of heteroaryl include, but are not limited to, thiopheneyl, furanyl, pyrrolyl, imidazolyl, pyrazolyl, thiazolyl, isothiazolyl, oxazolyl, isoxazolyl, triazolyl, tetrazolyl, pyridinyl, pyridazinyl, pyrazinyl, pyrimidinyl, quinolinyl, isoquinolinyl, indolyl, quinoxalinyl, benzo[b]thienyl, benzoxazolyl, benzimidazolyl, benzothiazolyl. Examples of substituted heteroaryl include, but are not limited to, 2-methyl-7-quinolinyl, 2-methyl-6-quinolinyl, 3-methyl-7-quinolinyl, 3-methyl-6-quinolinyl, 2-methoxy-6-quinolinyl, 2-methoxy-7-quinolinyl, 3-methoxy-6-quinolinyl, 3-methoxy-7-quinolinyl, 2-chloro-6-quinolinyl, 2-chloro-7-quinolinyl, 3-chloro-6-quinolinyl, 3-chloro-7-quinolinyl, 2-fluoro-6-quinolinyl, 2-fluoro-7-quinolinyl, 3-fluoro-6-quinolinyl, 3-fluoro-7-quinolinyl, 2-fluoromethyl-6-quinolinyl, 2-fluoromethyl-7-quinolinyl, 3-fluoromethyl-6-quinolinyl, 3-fluoromethyl-7-quinolinyl, 2-(3-hydroxypropyl)-7-quinolinyl, 2-(3-hydroxypropyl)-6-quinolinyl, 2-acetyl-6-quinolinyl, 2-acetyl-7-quinolinyl, 2-(4-thiazolylmethyl)-6-quinolinyl, 2-(4-thiazolylmethyl)-7-quinolinyl, 2-acetamidyl-7-quinolinyl, 2-acetamidyl-6-quinolinyl, 2-(2-acetoxyethyl)-7-quinolinyl, 2-(2-acetoxyethyl)-6-quinolinyl, 6-methoxy-2-pyrimidinyl, 5-methoxy-2-pyrimidinyl, 4-methoxy-2-pyrimidinyl, 5-chloro-2-pyridyl, 4-methoxy-2-pyridyl, 5-fluoro-2-pyridyl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, and 1-(2-methoxy-2-oxoethyl)-6-indolyl.

The term “heteroarylene” as used herein refers to divalent aromatic cyclic or polycyclic ring systems having from 1 to 4 heteroatoms independently selected from N, O, and S. Heterorylene groups may be unsubstituted or substituted with those substituents enumerated for aryl. Examples of heteroarylene groups include, but are not limited to, furan-2,5-diyl, thiophene-2,4-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, and pyrimidine-2,5-diyl.

The term “alkoxy” as used herein refers to —O-alkyl groups where “alkyl” is defined above.

An “effective amount” is an amount of a compound of the present invention that when administered to a patient ameliorates a symptom of disorders associated with renin activity such as hypertension and congestive heart failure. A therapeutically effective amount of a compound of the present invention can be easily determined by one skilled in the art by administering a quantity of a compound to a patient and observing the result. In addition, those skilled in the art are familiar with identifying patients having disorders associated with renin activity such as hypertension and congestive heart failure.

The term “treating” as used herein refers to the administration of a compound of Formula I, Formula II or pharmaceutically acceptable salts thereof that eliminates, alleviates, inhibits the progression of, or reverses progression of, in part or in whole, any one or more of the pathological hallmarks or symptoms of any one of the diseases and disorders being treated, including, but not limited to, hypertension, congestive heart failure, stroke, myocardial infarction, glaucoma, and hyperaldosteronism.

The term “preventing” as used herein refers to the prophylactic administration of a compound of Formula I, Formula II or pharmaceutically acceptable salts thereof to an asymptomatic patient at risk for the disease or disorder being prevented to inhibit the onset of an associated pathological hallmark or symptom, including, but not limited to, hypertension, congestive heart failure, stroke, myocardial infarction, glaucoma, and hyperaldosteronism. Additionally, once the onset of a pathological hallmark or symptom has begun, preventing means the prevention of further progression or reversal of progression, in part or in whole, of the pathological hallmark or symptom.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed.

The present invention provides compounds capable of inhibiting renin. Compounds of the present invention are described by Formula I:

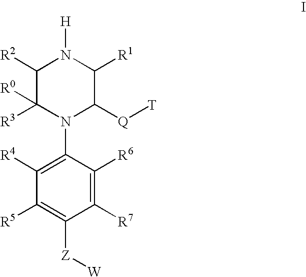

or a pharmaceutically acceptable salt thereof, where

R¹ and R² are independently hydrogen or unsubstituted C₁-C₃ alkyl;

R³ is hydrogen, oxo, or thioxo;

R⁰ is hydrogen or unsubstituted C₁-C₃ alkyl provided that when R³ is oxo or thioxo R⁰ is absent;

R⁴, R⁵, R⁶, and R⁷ are independently hydrogen, halogen, carboxyl, substituted or unsubstituted C₁-C₃ alkoxy, or substituted or unsubstituted C₁-C₃ alkyl;

Q is —(CH₂)₁₋₆—C(O)—O—(CH₂)₀₋₆—, —(CH₂)₁₋₆—O—C(O)—(CH₂)₀₋₆—, —(CH₂)₁₋₆—C(O)—NR⁸—(CH₂)₀₋₆—, —(CH₂)₁₋₆—NR⁹—C(O)—(CH₂)₀₋₆—, —(CH₂)₁₋₆—NR¹⁰—S(O)₂—(CH₂)₀₋₆—, —(CH₂)₁₋₆—S(O)₂—NR¹⁰—(CH₂)₀₋₆—, —(CH₂)₁₋₆—NR¹¹—C(O)—NR¹²—(CH₂)₀₋₆—, or —CH₂—(C₁-C₆ alkylene) where 1 to 3 nonadjacent methylene units of the alkylene group are replaced with O, NR¹³, S or a combination thereof;

T is substituted or unsubstituted aryl, substituted or unsubstituted heteroaryl, or substituted or unsubstituted C₁-C₁₂ alkyl;

W is absent, substituted or unsubstituted aryl, or substituted or unsubstituted heteroaryl;

Z is —(CH₂)₀₋₆-cycloalkylene-(CH₂)₀₋₆— where 0 to 6 nonadjacent methylene units are replaced with O, NR¹⁶, S or a combination thereof,

—(CH₂)₀₋₆-heterocycloalkylene-(CH₂)₀₋₆— where 0 to 6 nonadjacent methylene units are replaced with O, NR¹⁶, S or a combination thereof,

—(CH₂)₀₋₆-arylene-(CH₂)₀₋₆— where 0 to 6 nonadjacent methylene units are replaced with O, NR¹⁶, S or a combination thereof,

—(CH₂)₀₋₆-heteroarylene-(CH₂)₀₋₆— where 0 to 6 nonadjacent methylene units are replaced with O, NR¹⁶, S or a combination thereof,

—(CH₂)₀₋₆—C(O)—NR¹⁶—(CH₂)₀₋₆— where 0 to 6 nonadjacent methylene units are replaced with O, NR¹⁶, S or a combination thereof,

—(CH₂)₀₋₆—NR¹⁶—C(O)—(CH₂)₀₋₆— where 0 to 6 nonadjacent methylene units are replaced with O, NR¹⁶, S or a combination thereof,

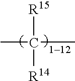

where 1 to 6 nonadjacent

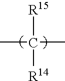

units are replaced with O, NR¹⁶,

S or a combination thereof, or

Z, when W is absent, is hydroxyl, substituted or unsubstituted C₁-C₁₂ alkyl where 1 to 6 nonadjacent methylene units are replaced with O,

NR¹⁶, S or a combination thereof, or —(CH₂)₀₋₆—C(O)—NR¹⁶—(CH₂)₀₋₅—CH₃ where 0 to 6 nonadjacent methylene units are replaced with O,

NR¹⁶, S or a combination thereof;

R⁸, R⁹, R¹⁰, R¹¹, and R¹² are independently hydrogen, substituted or unsubstituted C₁-C₃ alkoxy, or substituted or unsubstituted C₁-C₃ alkyl;

R¹³ and R¹⁶ are independently substituted or unsubstituted C₁-C₃ alkyl or hydrogen; and

R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl where 1 to 6 nonadjacent methylene units are replaced with O, or R¹⁴ and R¹⁵ together with the carbon to which they are attached form a 3- to 6-membered cycloalkylene or heterocycloalkylene ring.

Examples of compounds of Formula I include those where R¹ and R², are hydrogen and R³ is oxo.

Other examples of compounds of Formula I include those where R⁴, R⁵, R⁶, and R⁷ are independently hydrogen, halogen such as chlorine or fluorine, carboxyl, C₁-C₃ alkoxy such as methoxy, or C₁-C₃ alkyl such as methyl.

Other examples of compounds of Formula I include those where R⁴, R⁶, and R⁷ are hydrogen and R⁵ is chlorine, fluorine, carboxyl, methoxy or methyl

Other examples of compounds of Formula I include those where R⁴, R⁶, and R⁷ are hydrogen and R⁵ is chlorine, fluorine, carboxyl, methoxy or methyl.

Other examples of compounds of Formula I include those where Q is —(CH₂)₁₋₆—O—C(O)—(CH₂)₀₋₆—, or —CH₂—(C₁-C₆ alkylene) where 1 to 3 nonadjacent methylene units of the alkylene group are replaced with O, NR¹³, S or a combination thereof.

Additional examples of compounds of Formula I include those where Q is —CH₂—(C₁-C₆ alkylene) where 1 to 3 nonadjacent methylene units of the alkylene group are replaced with O or S.

Additional examples of compounds of Formula I include those where Q is —CH₂—O—, —CH₂—O—CH₂—CH₂—, —CH₂—O—CH₂—CH₂—CH₂—, —CH₂—S—, or —CH₂—O—C(O)—(CH₂)₀₋₆—.

Additional examples of compounds of Formula I include those where T is unsubstituted phenyl, naphthyl such as 2-naphthyl, biphenyl such as biphen-4-yl, 1,2,3,4-tetrahydroquinolinyl such as 1,2,3,4-tetrahydroquinolin-6-yl or 1,2,3,4-tetrahydroquinolin-7-yl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl.

Additional examples of compounds of Formula I include those where T is substituted phenyl, naphthyl, biphenyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

Additional examples of compounds of Formula I include those where T is phenyl substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof. For example compounds of Formula I where T is phenyl substituted from 1 to 5 times as stated above include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, and 4-acetylaminophenyl.

Additional examples of compounds of Formula I include those where T is biphenyl substituted from 1 to 9 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Additional examples of compounds of Formula I include those where T is naphthyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl substituted from 1 to 7 times with C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof. Examples of such compounds include 6-methoxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxyethyl)-2-naphthyl, 7-(2-acetoxyethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, and 1-(2-acetoxyethyl)-3,4-dihydro-2H-quinolin-7-yl.

Additional examples of compounds of Formula I include those where T is unsubstituted naphthyl, 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl. Further examples of compounds of Formula I include those where T is unsubstituted heteroaryl such as quinolinyl, indolyl, isoquinolinyl, pyridyl, pyrimidinyl, pyrazinyl, and quinoxalinyl. Examples of compounds of Formula I where T is unsubstituted heteroaryl include 2-quinolinyl, 6-quinolinyl, 7-quinolinyl, 6-isoquinolinyl, 2-pyridyl, 2-pyrimidinyl, 2-pyrazinyl, and 2-quinoxalinyl.

Further examples of compounds of Formula I include those where T is substituted heteroaryl such as substituted quinolinyl, indolyl, isoquinolinyl, pyridyl, pyrimidinyl, pyrazinyl, and quinoxalinyl. Examples of compounds of Formula I where T is substituted heteroaryl include quinolinyl, isoquinolinyl, or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl, or a combination thereof. Other examples include pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Further examples of compounds of Formula I include those where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl. Examples of compounds of Formula I where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl include those where the N-substituent is C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl. Additional N substituents include —(CH₂)₀₋₆—C(O)—O—(CH₂)₀₋₆-L, —(CH₂)₀₋₆—O—C(O)—(CH₂)₀₋₆-L, —(CH₂)₀₋₆—C(O)—NH—(CH₂)₀₋₆-L, —(CH₂)₀₋₆—NH—C(O)—(CH₂)₀₋₆-L, —(CH₂)₀₋₆—NH—S(O)₂—(CH₂)₀₋₆-L, —(CH₂)₀₋₆—S(O)₂—NH—(CH₂)₀₋₆-L, —(CH₂)₀₋₆—NH—C(O)—NH—(CH₂)₀₋₆-L, or —CH₂—(C₁-C₆ alkylene)-L where 1 to 3 nonadjacent methylene units of the alkylene group are replaced with O, NH, S or a combination thereof and where L is aryl, heteroaryl, or heterocycloalkyl.

Further examples of compounds of Formula I include those where Z is

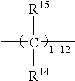

where 1 to 6 nonadjacent

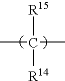

units are replaced with O.

Further examples of compounds of Formula I include those where R¹⁴ and R¹⁵ are hydrogen.

Further examples of compounds of Formula I include those where Z is

—(CH₂)₀₋₆—C(O)—NR¹⁶—(CH₂)₀₋₆— where 0 to 6 nonadjacent methylene units are replaced with O, NR¹⁶, S or a combination thereof; or

—(CH₂)₀₋₆—NR¹⁶—(C(O)—CH₂)₀₋₆— where 0 to 6 nonadjacent methylene units are replaced with O, NR¹⁶, S or a combination thereof; and

R¹⁶ is as defined above.

Further examples of compounds of Formula I include those where Z is —O—(CH₂)₂₋₃—O—(CH₂)₁₋₂— such as —O—(CH₂)₃—O—(CH₂)—, —O—(CH₂)₃₋₄—O—, O—(CH₂)₁₋₂—, —(CH₂)—O—(CH₂)₂₋₃—O—(CH₂)₀₋₁—, —C(O)—NR¹⁶—(CH₂)₂—, —C(O)—NR¹⁶—(CH₂)₂—O—, or —O—(CH₂)₃—S—(CH₂)₁—.

Further examples of compounds of Formula I include those where when W is absent, Z is hydroxyl, C₁-C₁₂ alkyl where 1 to 6 nonadjacent methylene units are replaced with O, or —(CH₂)₀₋₆—C(O)—NR¹⁶—(CH₂)₀₋₅—CH₃ where 0 to 6 nonadjacent methylene units are replaced with O.

Yet further examples of compounds of Formula I include those where W is unsubstituted or substituted phenyl. Examples of compounds of Formula I where W is substituted phenyl include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, and 4-acetylaminophenyl.

Yet further examples of compounds of Formula I include those where W is unsubstituted or substituted heteroaryl. Examples of compounds of Formula I where W is unsubstituted heteroaryl include indolyl such as 1H-Indol-3-yl.

Yet further examples of compounds of Formula I include those where Z is —O—(CH₂)₃—O—CH₂—, and W is 2-methoxyphenyl.

Yet further examples of compounds of Formula I include those where Q is —CH₂—O— or —CH₂—O—CH₂— and T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxyethyl)-3,4-dihydro-2H-quinolin-7-yl.

Still further examples of compounds of Formula I include those of Formula II:

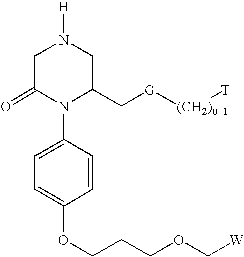

or a pharmaceutically acceptable salt thereof, where

G is O or S;

T is substituted or unsubstituted aryl, or substituted or unsubstituted heteroaryl;

W is substituted or unsubstituted aryl, or substituted or unsubstituted heteroaryl.

Other examples of compounds of Formula II include those where T is substituted aryl.

Other examples of compounds of Formula II include those where T is phenyl substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof. For example compounds of Formula I where T is phenyl substituted from 1 to 5 times as stated above include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, and 4-acetylaminophenyl.

Other examples of compounds of Formula II include those where T is substituted phenyl, naphthyl, biphenyl, 1,2,3,4-tetrahydroquinolinyl, 2-oxo-1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl. Examples of such compounds include 6-methoxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxyethyl)-2-naphthyl, 7-(2-acetoxyethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, and 1-(2-acetoxyethyl)-3,4-dihydro-2H-quinolin-7-yl.

Other examples of compounds of Formula II include those where T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Other examples of compounds of Formula II include those where T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

Additional examples of compounds of Formula II include those where T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Further examples of compounds of Formula II include those where T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Yet further examples of compounds of Formula II include those where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

Yet further examples of compounds of Formula II include those where T is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C—C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶ —C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl. Additional examples of compounds of Formula II include those where W is unsubstituted or substituted phenyl. Examples of compounds of Formula II where W is substituted phenyl include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, or 4-acetylaminophenyl.

Additional examples of compounds of Formula II include those where W is 2-methoxyphenyl.

Additional examples of compounds of Formula II include those where T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxyethyl)-3,4-dihydro-2H-quinolin-7-yl and W is 2-methoxyphenyl.

Representative compounds of Formula I include

(6S)-1-{4-[3-(2-methoxybenzyloxy)-propoxy]-phenyl}-6-(naphthalen-2-yloxymethyl)-piperazin-2-one;

(6R)-6-(3,4-dichlorobenzyloxymethyl)-1-{4-[3-(2-methoxybenzyloxy)-propoxy]-phenyl}-piperazin-2-one;

(6R)-6-(2-fluorobenzyloxymethyl)-1-{4-[3-(2-methoxybenzyloxy)-propoxy]-phenyl}-piperazin-2-one;

(6R)-6-(3,4-difluorobenzyloxymethyl)-1-{4-[3-(2-methoxybenzyloxy)-propoxy]-phenyl}-piperazin-2-one;

(6R)-6-(4-chlorobenzyloxymethyl)-1-{4-[3-(2-methoxybenzyloxy)-propoxy]-phenyl}-piperazin-2-one;

(6R)-6-(3-chlorobenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(4-methylbenzyloxymethyl)-piperazin-2-one;

(6R)-6-(4-fluorobenzyloxymethyl)-1-{4-[3-(2-methoxybenzyloxy)-propoxy]-phenyl}-piperazin-2-one;

(6R)-6-(3-methoxybenzyloxymethyl)-1-{4-[3-(2-methoxybenzyloxy)-propoxy]-phenyl}-piperazin-2-one;

(6R)-6-(2-methoxybenzyloxymethyl)-1-{4-[3-(2-methoxybenzyloxy)-propoxy]-phenyl}-piperazin-2-one;

(6R)-6-(3,5-difluorobenzyloxymethyl)-1-{4-[3-(2-methoxybenzyloxy)-propoxy]-phenyl}-piperazin-2-one;

(6R)-6-(4-methoxybenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(4-trifluoromethylbenzyloxymethyl)-piperazin-2-one;

(6R)-6-(2-chlorobenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(3-methylbenzyloxymethyl)-piperazin-2-one;

(6R)-6-(2,6-difluorobenzyloxymethyl)-1-{4-[3-(2-methoxybenzyloxy)-propoxy]-phenyl}-piperazin-2-one;

(6R)-6-(2,6-dichlorobenzyloxymethyl)-1-{4-[3-(2-methoxybenzyloxy)-propoxy]-phenyl}-piperazin-2-one;

(6R)-6-(3-fluorobenzyloxymethyl)-1-{4-[3-(2-methoxybenzyloxy)-propoxy]-phenyl}-piperazin-2-one;

(6R)-6-(4-fluoro-2-trifluoromethylbenzyloxymethyl)-1-{4-[3-(2-methoxybenzyloxy)-propoxy]-phenyl}-piperazin-2-one;

(6R)-6-(3,5-dichlorobenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(2-methylbenzyloxymethyl)-piperazin-2-one;

(6R)-6-(2-chloro-4-fluorobenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(pyridin-3-ylmethoxymethyl)-piperazin-2-one;

(6R)-6-(4-chloro-3-trifluoromethylbenzyloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(pyridin-4-ylmethoxymethyl)-piperazin-2-one;

(6R)-6-(4-fluoro-3-trifluoromethylbenzyloxymethyl)-1-{4-[3-(2-methoxybenzyloxy)-propoxy]-phenyl}-piperazin-2-one;

(6R)-6-(4-fluoro-3-methylbenzyloxymethyl)-1-{4-[3-(2-methoxybenzyloxy)-propoxy]-phenyl}-piperazin-2-one;

(6R)-4-(1-{4-[3-(2-methoxybenzyloxy)-propoxy]-phenyl}-6-oxopiperazin-2-ylmethoxymethyl)-benzonitrile;

(6R)-1-{4-[3-(2-methoxybenzyloxy)-propoxy]-phenyl}-6-(pyridin-2-ylmethoxymethyl)-piperazin-2-one;

(6R)-6-(4-bromobenzyloxymethyl)-1-{4-[3-(2-methoxybenzyloxy)-propoxy]-phenyl}-piperazin-2-one;

(2R)-1-{4-[3-(2-methoxybenzyloxy)-propoxy]-phenyl}-2-(naphthalen-2-yloxymethyl)-piperazine;

(2R)-2-(4-methoxybenzyloxymethyl)-1-{4-[3-(2-methoxy-benzyloxy)-propoxy]-phenyl}-piperazine;

(2R)-1-[4-(3-benzyloxypropoxy)-phenyl]-2-(4-methoxybenzyloxymethyl)-piperazine;

(2R)-1-(4-benzyloxyphenyl)-2-(naphthalen-2-yloxymethyl)-piperazine;

(2R)-1-(4-benzyloxyphenyl)-2-(4-methoxybenzyloxymethyl)-piperazine;

(6R)-1-{4-[3-(2-methoxybenzyloxy)-propoxy]-phenyl}-6-(naphthalen-2-yloxymethyl)-piperazin-2-one;

(2R)-1-[4-(3-benzyloxypropoxy)-phenyl]-2-(naphthalen-2-yloxymethyl)-piperazine;

(6R)-1-{3-fluoro-4-[3-(2-methoxybenzyloxy)-propoxy]-phenyl}-6-(naphthalen-2-yloxymethyl)-piperazin-2-one;

(2R)-1-{4-[3-(2-methoxybenzyloxy)-propoxy]-phenyl}-2-(5,6,7,8-tetrahydronaphthalen-2-yloxymethyl)-piperazine;

(6R)-1-{4-[3-(2-methoxybenzyloxy)-propoxy]-phenyl}-6-(quinolin-7-yloxymethyl)-piperazin-2-one;

(6R)-1-{4-[3-(2-methoxybenzyloxy)-propoxy]-phenyl}-6-(1,2,3,4-tetrahydroquinolin-7-yloxymethyl)-piperazin-2-one;

(6R)-1-{3,5-difluoro-4-[3-(2-methoxybenzyloxy)-propoxy]-phenyl}-6-(naphthalen-2-yloxymethyl)-piperazin-2-one;

(6R)-6-[1-(3-hydroxypropyl)-1,2,3,4-tetrahydroquinolin-7-yloxymethyl-1-{4-[3-(2-methoxybenzyloxy)-propoxy]-phenyl}-piperazin-2-one;

(6R)-6-benzyloxymethyl-1-{4-[3-(2-methoxybenzyloxy)-propoxy]-phenyl}-piperazin-2-one;

(6S)-6-(4-fluorobenzyloxymethyl)-1-{4-[3-(2-methoxybenzyloxy)-propoxy]-phenyl}-piperazin-2-one;

4-[(2R)-2-(naphthalen-2-yloxymethyl)-6-oxopiperazin-1-yl]-N-phenethylbenzamide;

(6R)-1-[4-methoxy-3-(3-methoxypropoxy)-phenyl]-6-(naphthalen-2-yloxymethyl)-piperazin-2-one;

N-(2-ethoxyethyl)-4-[(2R)-2-(naphthalen-2-yloxymethyl)-6-oxopiperazin-1-yl]-benzamide;

N-[2-(3-methoxyphenyl)-ethyl]-4-[(2R)-2-(naphthalen-2-yloxymethyl)-6-oxopiperazin-1-yl]-benzamide;

(6R)-6-(isoquinolin-7-yloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(quinolin-6-yloxymethyl)-piperazin-2-one;

4-[(2R)-2-(naphthalen-2-yloxymethyl)-6-oxopiperazin-1-yl]-N-(2-phenoxyethyl)-benzamide;

(6R)-6-(1-acetyl-1,2,3,4-tetrahydroquinolin-6-yloxymethyl)-1-{4-[3-(2-methoxybenzyloxy)-propoxy]-phenyl}-piperazin-2-one;

(6R)-1-{4-[3-(2-methoxybenzyloxy)-propoxy]-phenyl}-6-(1-thiazol-4-ylmethyl-1,2,3,4-tetrahydroquinolin-7-yloxymethyl)-piperazin-2-one;

2-[7-(1-{4-[3-(2-methoxybenzyloxy)-propoxy]-phenyl}-6-oxopiperazin-2R-ylmethoxy)-3,4-dihydro-2H-quinolin-1-yl]-acetamide;

(6R)-6-[1-(2-hydroxyethyl)-1,2,3,4-tetrahydroquinolin-7-yloxymethyl]-1-{4-[3-(2-methoxybenzyloxy)-propoxy]-phenyl}-piperazin-2-one;

naphthalene-2-carboxylic acid (2R)-1-{4-[3-(2-methoxy-benzyloxy)-propoxy]-phenyl}-6-oxo-piperazin-2-yl methyl ester;

4-methyl-benzoic acid (2R)-1-{4-[3-(2-methoxy-benzyloxy)-propoxy]-phenyl}-6-oxo-piperazin-2-yl methyl ester;

4-chloro-benzoic acid (2R)-1-{4-[3-(2-methoxy-benzyloxy)-propoxy]-phenyl}-6-oxo-piperazin-2-yl methyl ester;

benzoic acid (2R)-1-{4-[3-(2-methoxybenzyloxy)-propoxy]-phenyl}-6-oxopiperazin-(2R)-yl methyl ester;

(2R)-1-{4-[3-(4-chlorobenzyloxy)-propoxy]-phenyl}-2-(4-methoxybenzyloxymethyl)-piperazine;

(2R)-1-{4-[3-(3,4-dichlorobenzyloxy)-propoxy]-phenyl}-2-(4-methoxybenzyloxymethyl)-piperazine;

(2R)-1-{4-[3-(3-chlorobenzyloxy)-propoxy]-phenyl}-2-(4-methoxybenzyloxymethyl)-piperazine;

(2R)-2-(4-methoxybenzyloxymethyl)-1-{4-[3-(4-methoxybenzyloxy)propoxy]-phenyl}-piperazine;

(2R)-1-{4-[3-(2-chlorobenzyloxy)-propoxy]-phenyl}-2-(4-methoxybenzyloxymethyl)-piperazine;

(2R)-1-{4-[3-(3,5-difluorobenzyloxy)-propoxy]-phenyl}-2-(4-methoxybenzyloxymethyl)-piperazine;

(2R)-2-(4-methoxybenzyloxymethyl)-1-{4-[3-(4-methylbenzyloxy)propoxy]-phenyl}-piperazine;

(2R)-2-(4-methoxybenzyloxymethyl)-1-{4-[3-(3-methoxybenzyloxy)-propoxy]-phenyl}-piperazine;

(2R)-2-(4-methoxybenzyloxymethyl)-1-{4-[3-(2-methoxyphenoxy)-propoxymethyl]-phenyl}-piperazine;

(6R)-6-(4-fluorobenzyloxymethyl)-1-{4-[4-(2-methoxyphenoxy)-butoxy]-phenyl}-piperazin-2-one;

(2R)-1-{4-[2-(2-methoxybenzyloxy)-ethoxymethyl]-phenyl}-2-(4-methoxybenzyloxymethyl)-piperazine;

(6R)-1-{4-[4-(2-methoxyphenoxy)-butoxy]-phenyl}-6-(naphthalen-2-yloxymethyl)-piperazin-2-one;

(6R)-6-(4-fluorobenzyloxymethyl)-1-{4-[2-(2-methoxybenzyloxy)-ethoxymethyl]-phenyl}-piperazin-2-one;

(2R)-2-(4-methoxybenzyloxymethyl)-1-{4-[4-(2-methoxyphenoxy)-butoxy]-phenyl}-piperazine;

(6R)-6-(4-fluorobenzyloxymethyl)-1-{4-[3-(2-methoxyphenoxy)-propoxymethyl]-phenyl}-piperazin-2-one;

(6R)-1-{4-[3-(2-methoxybenzyloxy)-propoxy]-phenyl}-6-(quinolin-2-yloxymethyl)-piperazin-2-one;

(6R)-1-{4-[3-(2-methoxybenzyloxy)-propoxy]-phenyl}-6-(quinoxalin-2-yloxymethyl)-piperazin-2-one;

(6R)-1-{4-[3-(2-methoxybenzyloxy)-propoxy]-phenyl}-6-(pyrazin-2-yloxymethyl)-piperazin-2-one;

(6R)-1-{4-[3-(2-methoxybenzyloxy)-propoxy]-phenyl}-6-(pyridin-2-yloxymethyl)-piperazin-2-one;

(6R)-1-{4-[3-(2-methoxybenzyloxy)-propoxy]-phenyl}-6-(pyrimidin-2-yloxymethyl)-piperazin-2-one;

2-methoxy-N-([2R]-1-{4-[3-(2-methoxybenzyloxy)-propoxy]-phenyl}-6-oxo-piperazin-2-ylmethyl)-benzamide;

4-chloro-N-([2R]-1-{4-[3-(2-methoxy-benzyloxy)-propoxy]-phenyl}-6-oxo-piperazin-2-ylmethyl)-benzamide;

N-([2R]-1-{4-[3-(2-methoxy-benzyloxy)-propoxy]-phenyl}-6-oxo-piperazin-2-ylmethyl)-benzamide;

naphthalene-2-carboxylic acid ([2R]-1-{4-[3-(2-methoxy-benzyloxy)-propoxy]-phenyl}-6-oxo-piperazin-2-ylmethyl)-amide;

2-fluoro-N-([2R]-1-{4-[3-(2-methoxy-benzyloxy)-propoxy]-phenyl}-6-oxo-piperazin-2-ylmethyl)-benzamide;

(2R)-1-{4-[3-(2-fluorobenzyloxy)-propoxy]-phenyl}-2-(naphthalen-2-yloxymethyl)-piperazine;

(2R)-1-{4-[3-(2-ethoxybenzyloxy)-propoxy]-phenyl}-2-(naphthalen-2-yloxymethyl)-piperazine;

(2R)-1-{4-[3-(3-methoxybenzyloxy)-propoxy]-phenyl}-2-(naphthalen-2-yloxymethyl)-piperazine;

(2R)-1-{4-[3-(2-methoxybenzyloxy)-propoxy]-phenyl}-2-(naphthalen-2-ylmethoxymethyl)-piperazine;

(2R)-2-(biphenyl-3-ylmethoxymethyl)-1-{4-[3-(2-methoxybenzyloxy)-propoxy]-phenyl}-piperazine;

(6R)-6-(biphenyl-4-yloxymethyl)-1-(4-[3-(2-methoxybenzyloxy)-propoxy]-phenyl)-piperazin-2-one;

N-[4-([2R]-1-{4-[3-(2-methoxybenzyloxy)-propoxy]-phenyl}-6-oxopiperazin-2-ylmethoxy)-phenyl]-acetamide;

(2R)-1-{4-[2-(2-methoxybenzyloxy)-ethoxymethyl]-phenyl}-2-(naphthalene-2-yloxymethyl)-piperazine;

4-([2R]-1-{4-[3-(2-methoxy-benzyloxy)-propoxy]-phenyl}-6-oxo-piperazin-2-ylmethoxy)-N,N-dimethyl-benzamide;

2-[(5R)-3-(2-methoxybenzyloxy)-propoxy]-5-[2-(naphthalene-2-yloxymethyl)-6-oxopiperazin-1-yl]-benzoic acid methyl ester;

(2R)-1-(4-[3-(2-methoxyphenoxy)-propoxymethyl]-phenyl)-2-(naphthalene-2-yloxymethyl)-piperazine;

2-[(5R)-[3-(2-methoxybenzyloxy)-propoxy]-5-[2-(naphthalene-2-yloxymethyl)-6-oxopiperazin-1-yl]-benzoic acid;

(2R)-1-(4-methoxymethylphenyl)-2-(naphthalene-2-yloxymethyl)-piperazine;

(6R)-1-(3-chloro-4-[3-(2-methoxybenzyloxy)-propoxy]-phenyl)-6-(naphthalene-2-yloxymethyl)-piperazin-2-one;

(6R)-1-{4-[3-(2-methoxybenzylsulfanyl)-propoxy]-phenyl}-6-(naphthalene-2-yloxymethyl)-piperazin-2-one;

(2R)-1-{4-[3-(2-methoxybenzylsulfanyl)-propoxy]-phenyl}-2-(naphthalene-2-yloxymethyl)-piperazine;

(6R)-1-{4-[3-(2-methoxybenzyloxy)-propoxy]-3-methyl-phenyl}-6-(naphthalene-2-yloxymethyl)-piperazin-2-one;

(2R)-1-{4-{2-[2-(2-methoxyphenyl)-ethoxy]-ethoxy}-ethoxy}-phenyl)-2-(naphthalene-2-yloxymethyl)-piperazine;

(6R)-1-{4-[3-(2-methoxybenzyloxy)-propoxy]-phenyl}-6-(7-methoxynaphthalen-2-yloxymethyl)-piperazin-2-one;

(6R)-6-(biphenyl-3-yloxymethyl)-1-{4-[3-(2-methoxybenzyloxy)-propoxy]-phenyl}-piperazin-2-one;

(6R)-1-(3-methoxy-4-[3-(2-methoxybenzyloxy)-propoxy]-phenyl)-6-(naphthalene-2-yloxymethyl)-piperazin-2-one; or

(2R)-1-{4-[4-(2-methoxyphenoxy)-butoxy]-phenyl}-2-(naphthalene-2-yloxymethyl)-piperazine.

Additional representative compounds of Formula I include

[6-([2R]-1-{4-[3-(2-methoxybenzyloxy)-propoxy]-phenyl}-6-oxopiperazin-2-ylmethylsulfanyl)-3-oxo-2,3-dihydrobenzo[1,4]oxazin-4-yl]-acetic acid ethyl ester;

6-([2R]-1-{4-[3-(2-methoxybenzyloxy)-propoxy]-phenyl}-6-oxopiperazin-2-ylmethylsulfanyl)-4-(3-methoxypropyl)-4H-benzo[1,4]oxazin-3-one;

N-{2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl}-acetamide;

3-[7-([2R]-1-{4-[3-(2-methoxybenzyloxy)-propoxy]-phenyl}-6-oxopiperazin-2-ylmethoxy)-3,4-dihydro-2H-quinolin-1-yl]-propionic acid methyl ester;

acetic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester;

N-{2-[7-([2R]-1-{4-[3-(2-methoxybenzyloxy)-propoxy]-phenyl}-piperazin-2-ylmethoxy)-3,4-dihydro-2H-quinolin-1-yl]-ethyl}-acetamide;

3-[5-([2R]-1-{4-[3-(2-methoxybenzyloxy)-propoxy]-phenyl}-6-oxopiperazin-2-ylmethoxy)-indol-1-yl]-propionic acid methyl ester;

7-([2S]-1-{4-[3-(2-methoxybenzyloxy)-propoxy]-phenyl}-6-oxopiperazin-2-ylmethoxy)-1-(3-methoxypropyl)-3,4-dihydro-1H-quinolin-2-one;

(6R)-1-{4-[3-(2-methoxybenzyloxy)-propoxy]-phenyl}-6-[1-(3-methoxypropyl)-1,2,3,4-tetrahydroquinolin-7-yloxymethyl]-piperazin-2-one;

7-([2R] 1-{4-[3-(2-methoxybenzyloxy)-propoxy]-phenyl}-6-oxopiperazin-2-ylmethoxy)-1-(3-methoxypropyl)-3,4-dihydro-1H-quinolin-2-one;

[7-([2R]-1-{4-[3-(2-methoxybenzyloxy)-propoxy]-phenyl}-6-oxopiperazin-2-ylmethoxy)-2-oxo-3,4-dihydro-2H-quinolin-1-yl]-acetic acid methyl ester;

[7-([2R]-1-{4-[3-(2-methoxybenzyloxy)-propoxy]-phenyl}-6-oxopiperazin-2-ylmethoxy)-3,4-dihydro-2H-quinolin-1-yl]-acetic acid methyl ester;

N-{2-[5-([2R]-1-{4-[3-(2-methoxybenzyloxy)-propoxy]-phenyl}-6-oxopiperazin-2-ylmethoxy)-indol-1-yl]-ethyl}-acetamide;

N-{2-[7-([2R]-1-{4-[3-(2-fluorobenzyloxy)-propoxy]-phenyl}-6-oxopiperazin-2-ylmethoxy)-3,4-dihydro-2H-quinolin-1-yl]-ethyl}-acetamide;

[6-([2R]-1-{4-[3-(2-methoxybenzyloxy)-propoxy]-phenyl}-6-oxopiperazin-2-ylmethoxy)-indol-1-yl]-acetic acid ethyl ester;

[5-([2R]-1-{4-[3-(2-methoxybenzyloxy)-propoxy]-phenyl}-6-oxopiperazin-2-ylmethoxy)-2-methyl-indol-1-yl]-acetic acid methyl ester;

[6-([2R]-1-{4-[3-(2-methoxybenzyloxy)-propoxy]-phenyl}-6-oxopiperazin-2-ylmethoxy)-3-oxo-2,3-dihydrobenzo[1,4]oxazin-4-yl]-acetic acid methyl ester;

propionic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester;

3-[6-([2R]-1-{4-[3-(2-methoxybenzyloxy)-propoxy]-phenyl}-6-oxopiperazin-2-ylmethoxy)-2,3-dihydroindol-1-yl]-propionic acid methyl ester; and

[5-([2S]-1-{4-[3-(2-methoxybenzyloxy)-propoxy]-phenyl}-6-oxopiperazin-2-ylmethoxy)-indol-1-yl]-acetic acid methyl ester.

The compounds of Formula I have at least one asymmetric carbon atom, that being the piperazine or piperazinone carbon atom attached to the -Q-T moiety, and can exist in the form of optically pure enantiomers, racemates, diastereomer mixtures, diastereomeric racemates, or mixtures of diastereomeric racemates.

Processes and novel intermediates for preparing compounds of Formula I and II are provided as further embodiments of the invention and are illustrated by the following procedures in which the meanings of the generic radicals are as given above unless otherwise qualified. In some cases, protecting groups may have been used to allow synthetic manipulation of one functional group in the presence of other functional groups. It is therefore to be noted that, although not specifically noted in Schemes 1, 1a, 2, and 3the appropriate use and choice of protecting groups is well-known by one skilled in the art, and is not limited to the specific examples below. It is also to be understood that such groups not only serve to protect chemically reactive sites, but also to enhance solubility or otherwise change physical properties. A good general reference for protecting group preparation and deprotection is Greene, Theodora, Protective Groups in Organic Synthesis; Wiley: New York, USA, 1991.

Structures encompassed by Formula I where the phenyl ring attached to the piperazine ring is unsubstituted can be prepared as described in Scheme 1. The protected para-hydroxy aniline starting materials 1 are typically commercially available. For example, 4-benzyloxyaniline hydrochloride is commercially available from Aldrich Chemical Co., Milwaukee Wis. Thus, the amine of the protected para-hydroxy aniline 1, where P¹ is a suitable protecting group such as benzyl for example, is acylated to afford the intermediate 2, where R²⁰ is halo and R² is as defined above. Suitable acylating agents include alpha-halo acid chlorides such as chloroacetyl chloride and 2-chloropropionyl chloride for example. This acylation can be carried out in an art recognized solvent such as tetrahydrofuran for example. The intermediate 2 is then contacted with a suitable amine, such as benzylamine for example, resulting in amine substitution of the halide to afford the intermdiate 3, where P² is a suitable protecting group such as benzyl for example. This substitution can be carried out in an art recognized solvent such as tetrahydrofuran for example. The intermediate 3 is then contacted with a suitable epoxide to afford the intermediate 4, where R²¹ is halo and R¹ is as defined above. Suitable epoxides include epichlorohydrin, such as (S)-epichlorohydrin and (R)-epichlorohydrin. This epoxide amination can be carried out in an art recognized solvent such as an alcohol, for example methanol. The intermediate 4 is then cyclized in the presence of a base to afford the piperazinone intermediate 5. Suitable bases include those capable or deprotonating the aniline nitrogen and include the alkali metal hydroxides such as sodium hydroxide for example. This cyclization can be carried out in an art recognized solvent such as tetrahydrofuran, methanol, or a combination of the two. The protecting groups P¹ and P² are then removed from the piperazinone intermediate 5 using deprotection methods recognized in the art. The deprotection of intermediate 5 can be performed via hydrogenation using a suitable catalyst such as palladium on carbon for example. Deprotection of intermediate 5 is followed by protection of the piperazinone nitrogen with a suitable protecting group, P³, to afford the intermediate 6. Alternatively, the deprotection and protection of intermediate 5 can be performed simultaneously. Suitable P³ protecting groups include t-butyloxycarbonyl (BOC) for example. These deprotection and protection steps can be carried out in an art recognized solvent such as an alcohol, for example ethanol. The intermediate 6 is then alkylated with a suitable alkylating agent to afford the intermediate 7, where R²², along with the oxygen at the 4-position of the phenyl ring, is equivalent to -Z-W as is defined above in Formula I. Suitable alkylating agents include halo-R²², such as I—R²² and Cl—R²² for example, which can be prepared by those skilled in the art using known reagents and techniques. One example of a method for preparing suitable alkylating agents is described in Method E in the Examples section. Other examples of suitable alkylating agents include those where R²² is C₁-C₁₂ alkyl, benzyl, 4-trifluoromethylbenzyl, 3,4,5-trifluorobenzyl, 2-naphthylmethyl, 2-methoxybenzyloxypropyl, 3-methoxybenzyloxypropyl, 4-methoxybenzyloxypropyl, 2-fluorobenzyloxypropyl, benzyloxypropyl, 2-ethoxybenzyloxypropyl, 2-methoxybenzyloxyethyl, 2-methoxyphenoxybutyl, 2-methoxyphenoxypropyl, 3,5-difluorobenzyloxypropyl, 2-chlorobenzyloxypropyl, 3-chlorobenzyloxypropyl, 4-chlorobenzyloxypropyl, 3,4-dichlorobenzyloxypropyl, 4-phenylmethyl, 2-difluoromethoxybenzyl, 3-(2-fluorophenoxy)-benzyl, 2-(3-indolyl)ethyl, and 2-methoxybenzylthiopropyl. The alkylation of intermediate 6 can be carried out in an art recognized solvent such as acetonitrile for example. The intermediate 7 is then contacted with an appropriate alcohol to afford the desired ether 8, where R²³, along with the hydroxymethyl substituent of the piperazinone, is equivalent to -Q-T as is defined above in Formula I. Suitable alcohols can be prepared by those skilled in the art using known reagents and techniques. Suitable alcohols include 2-naphthol, 7-methoxy-2-naphthol, 2-hydroxymethylnaphthalene, 4-trifluoromethylbenzylalcohol, 7-hydroxy-1,2,3,4-tetrahydro-quinoline, 2-hydroxyquinoline, 7-hydroxymethylquinoline, benzylalcohol, 2-chlorobenzylalcohol, 3-chlorobenzylalcohol, 4-chlorobenzylalcohol, 2-fluorobenzylalcohol, 3-fluorobenzylalcohol, 4-fluorobenzylalcohol, 2-methylbenzylalcohol, 3-methylbenzylalcohol, 4-methylbenzylalcohol, 2-methoxybenzylalcohol, 3-methoxybenzylalcohol, 4-methoxybenzylalcohol, 3,4-dichlorobenzylalcohol, 3,4-difluorobenzylalcohol, 3,5-dichlorobenzylalcohol, 3,5-difluorobenzylalcohol, 7-hydroxymethyl-1,2,3,4-tetrahydro-quinoline, 1,2,3,4-tetrahydronaphth-7-ol, 6-isoquinolinol, 6-quinolinol, 7-quinolinol, 4-phenylbenzylalcohol, 3-phenylphenol, 4-hydroxy-N,N-dimethyl-benzamide, 2-hydroxypyridine, 2-hydroxypyrimidine, 2-hydroxypyrazine, 2-chloro-3-fluorobenzylalcohol, 1-(6-hydroxy-3,4-dihydro-2H-quinolin-1-yl)-ethanone, 1-thiazol-4-ylmethyl-1,2,3,4-tetrahydro-quinolin-7-ol, quinoxalin-2-ol, 4-fluoro-2-trifluoromethylbenzylalcohol, 2-(7-hydroxy-3,4-dihydro-2H-quinolin-1-yl)-acetamide, N-(4-hydroxymethyl-phenyl)-acetamide, and 1-(2-acetoxyethyl)-3,4-dihydro-2H-quinolin-7-ol. The conversion of intermediate 7 to intermediate 8 can take place using standard Mitsonobu conditions. Such conditions include carrying out the conversion in the presence of triphenylphophine and diisopropylazodicarboxylate in an art recognized solvent such as dichloromethane. Alternatively, the intermediate 7 can be converted to the desired ether 8 by contacting it with a benzyl bromide in the presence of sodium hydride and a phase transfer catalyst such as 18-crown-6 or 15-crown-5. An additional alternative for the preparation of ethers 8 involves the conversion of intermediate 7 to the triflate with trifluoromethanesulfonic anhydride, for example, followed by contact with an appropriate alcohol as defined above for the conversion to intermediate 8. The intermediate 8 is then deprotected to afford the final product 9 which corresponds to compounds of Formula I. Deprotection of intermediate 8 can be accomplished using deprotection methods recognized in the art. For example, the deprotection of intermediate 8 can be accomplished with acetyl chloride in an art recognized solvent such as methanol.

When piperazine and not piperazinones are desired (i.e. when R¹, R², and R³ are all hydrogen), intermediate 8 can be reduced to afford the desired piperazine prior to the final deprotection step. Such reductions are known to those of skill in the art and include the use of hydride reducing agents, such as lithium aluminum hydride, or other suitable reducing agents in an appropriate solvent such as tetrahydrofuran.

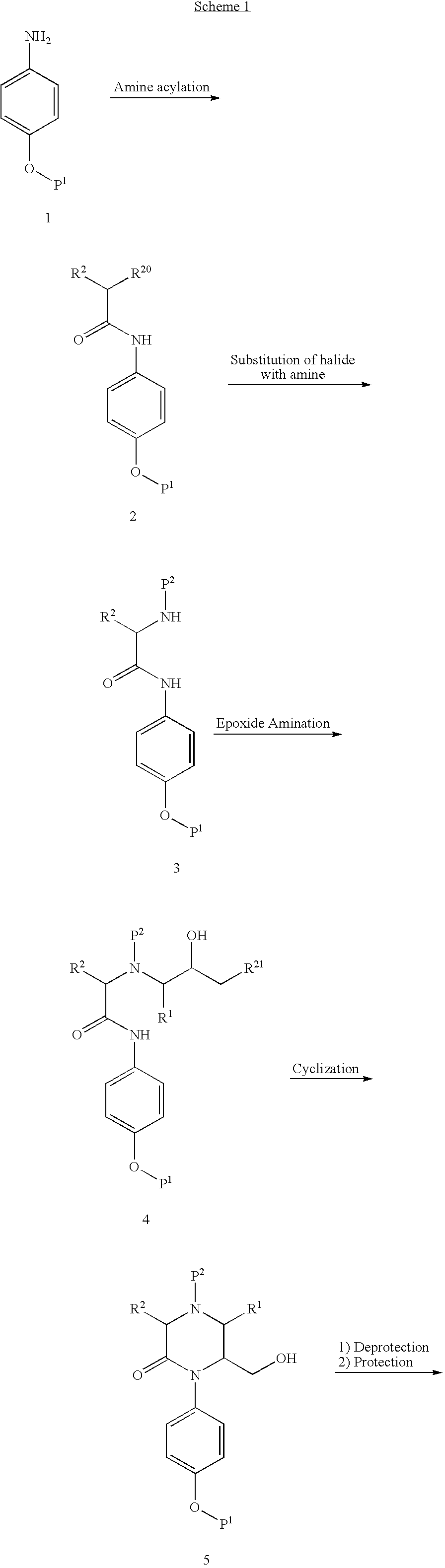

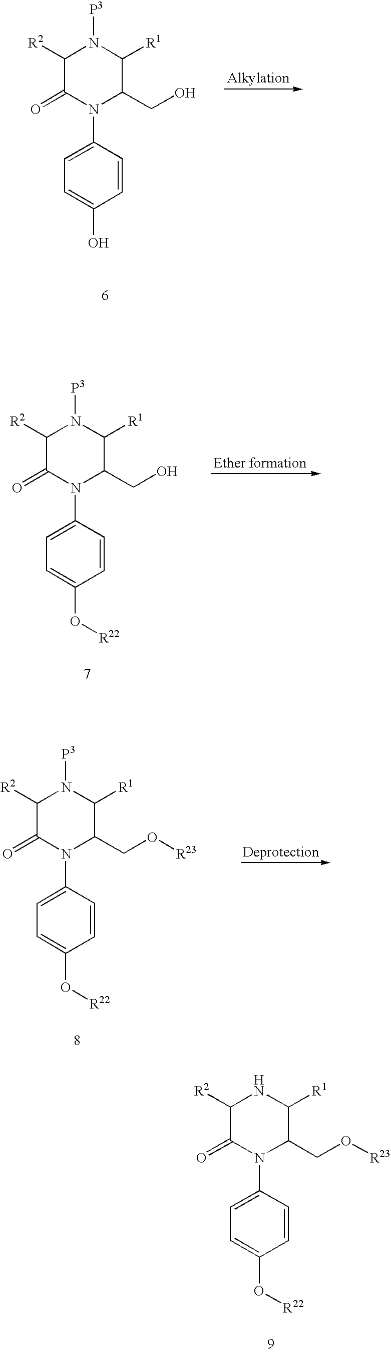

Structures encompassed by Formula I where an ester linkage is desired instead of an ether linkage can be prepared as described in Scheme 1a. The compound 7, prepared as stated in Scheme 1 and where R¹, R², P³, and R²² are as stated in Scheme 1, is then contacted with an appropriate acid chloride to afford the ester 7a where R^{23′}, along with the —CH₂—O—C(O)— moiety to which it is attached, is equivalent to -Q-T as is defined above in Formula I. Suitable acid chlorides include 2-naphthoyl chloride, benzoyl chloride, 4-chlorobenzoyl chloride, 3,4-dichlorobenzoyl chloride, 2-fluorobenzoyl chloride, 4-fluorobenzoyl chloride, 4-methylbenzoyl chloride, 2-methoxybenzoyl chloride, and 4-methoxybenzoyl chloride, for example. The intermediate ester 7a is then deprotected to afford the desired product 7b which corresponds to compounds of Formula I. For example, the deprotection of intermediate 7a can be accomplished with acetyl chloride in an art recognized solvent such as methanol.

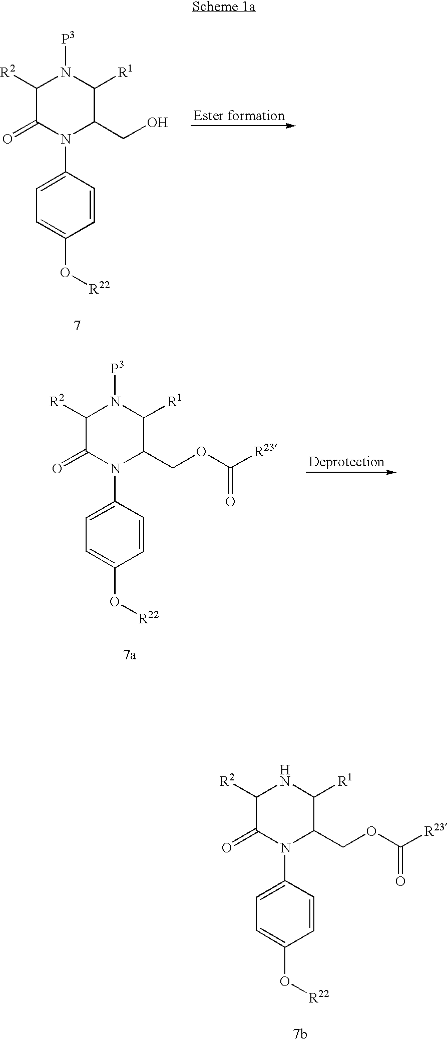

Structures encompassed by Formula I where the phenyl ring attached to the piperazine ring is substituted can be prepared as described in Scheme 2. The oxazolidine starting material 10, where P⁴ is a suitable amine protecting group such as t-butyloxycarbonyl (BOC) for example, is prepared and reduced to form the aldehyde 11 as disclosed in Org. Syn. 1992, 70, 18-28 and Org. Syn. 1992, 77, 64-77. Such reductions are known to those of skill in the art and include the use of hydride reducing agents, such as diisobutylaluminum hydride, or other suitable reducing agents at reduced temperatures from about −30° C. to about −80° C. in an appropriate solvent such as dichloromethane. The intermediate 11 is then aminated with an N-protected amino acid ester to afford 12, where P⁵ is a suitable amine protecting group, such as benzyl for example, and R¹ and R² are as describe above for Formula I. Suitable N-protected amino acid esters include N-protected glycine esters such as N-benzylglycine ethyl ester available from Aldrich Chemical Co. Milwaukee, Wis. The amination can be carried out in an art recognized solvent such as methanol and is typically carried out in the presence of a reducing agent such as sodium cyanoborohydride and an acid such as acetic acid for example. The intermediate 12 can then be cyclized to form the intermediate 13 upon treatment with acid, such as hydrochloric acid, at elevated temperatures from about 30° C. to about 80° C. in an appropriate solvent such as methanol. The protecting group P⁵ is then removed from the piperazinone intermediate 13 using deprotection methods recognized in the art. The deprotection of intermediate 13 can be performed via hydrogenation using a suitable catalyst such as palladium on carbon for example. Deprotection of intermediate 13 is followed by protection of the piperazinone nitrogen with a suitable protecting group, P⁶, to afford the intermediate 14. Suitable P⁶ protecting groups include t-butyloxycarbonyl (BOC) for example. These deprotection and protection steps can be carried out in an art recognized solvent such as an alcohol, for example ethanol. The intermediate 14 can be converted to the protected final product 18 through ether formation followed by amidation, path (a), or through amidation followed by ether formation, path (b). Regardless of order, the ether formation step can take place using standard Mitsonobu conditions. Such conditions include carrying contacting either intermediate 14 or 17 with an appropriate alcohol in the presence of triphenylphophine and diisopropylazodicarboxylate in an art recognized solvent such as dichloromethane to afford the desired ether 18, where R²⁴ is equivalent to -Q-T as is defined above in Formula I. Alternatively, the ether step can be accomplished by contacting either intermediate 14 or 17 with a benzyl bromide in the presence of sodium hydride and a phase transfer catalyst such as 18-crown-6 or 15-crown-5. An additional alternative for the preparation of ethers 16 and 18 involves the conversion of intermediates 14 or 17 to the triflate with trifluoromethanesulfonic anhydride, for example, followed by contact with an appropriate alcohol. Suitable alcohols can be prepared by those skilled in the art using known reagents and techniques. Suitable alcohols include 2-naphthol, 7-methoxy-2-naphthol, 2-hydroxymethylnaphthalene, 4-trifluoromethylbenzylalcohol, 7-hydroxy-1,2,3,4-tetrahydro-quinoline, 2-hydroxyquinoline, 7-hydroxymethylquinoline, benzylalcohol, 2-chlorobenzylalcohol, 3-chlorobenzylalcohol, 4-chlorobenzylalcohol, 2-fluorobenzylalcohol, 3-fluorobenzylalcohol, 4-fluorobenzylalcohol, 2-methylbenzylalcohol, 3-methylbenzylalcohol, 4-methylbenzylalcohol, 2-methoxybenzylalcohol, 3-methoxybenzylalcohol, 4-methoxybenzylalcohol, 3,4-dichlorobenzylalcohol, 3,4-difluorobenzylalcohol, 3,5-dichlorobenzylalcohol, 3,5-difluorobenzylalcohol, 7-hydroxymethyl-1,2,3,4-tetrahydro-quinoline, 1,2,3,4-tetrahydronaphth-7-ol, 6-isoquinolinol, 6-quinolinol, 7-quinolinol, 4-phenylbenzylalcohol, 3-phenylphenol, 4-hydroxy-N,N-dimethyl-benzamide, 2-hydroxypyridine, 2-hydroxypyrimidine, 2-hydroxypyrazine, 2-chloro-3-fluorobenzylalcohol, 1-(6-hydroxy-3,4-dihydro-2H-quinolin-1-yl)-ethanone, 1-thiazol-4-ylmethyl-1,2,3,4-tetrahydroquinolin-7-ol, quinoxalin-2-ol, 4-fluoro-2-trifluoromethylbenzylalcohol, 2-(7-hydroxy-3,4-dihydro-2H-quinolin-1-yl)-acetamide, N-(4-hydroxymethyl-phenyl)-acetamide, and 1-(2-acetoxyethyl)-3,4-dihydro-2H-quinolin-7-ol. Regardless of order, the amidation step includes contacting either intermediate 14 or 16 with an appropriate aryl halide 15 where R²⁵ is halo, such as iodo or bromo for example, R²⁶ is equivalent to -Z-W as defined above for Formula I, and R⁴, R⁵, R⁶, and R⁷ are as defined above for Formula I using conditions described by Klapars, A; Buchwald, S. J. Am. Chem. Soc. 2001, 123, 7727-7729 and Klapars, A; Buchwald, S. J. Am. Chem. Soc. 2002, 124, 7421-7428. Suitable aryl halides include 4-iodo-[(2-methoxybenzyloxy)-propoxy]-benzene, 2-fluoro-4-iodo-[(2-methoxybenzyloxy)-propoxy]-benzene, 4-iodo-2-methyl-[(2-methoxybenzyloxy)-propoxy]-benzene, 4-iodo-2-methoxy-[(2-methoxybenzyloxy)-propoxy]-benzene, 2,6-difluoro-4-iodo-[(2-methoxybenzyloxy)-propoxy]-benzene, 2-chloro-4-iodo-[(2-methoxybenzyloxy)-propoxy]-benzene, and 5-iodo-[(2-methoxybenzyloxy)-propoxy]-benzoic acid for example. The intermediate 18 is then deprotected to afford the final product 19 which corresponds to compounds of Formula I. Deprotection of intermediate 18 can be accomplished using deprotection methods recognized in the art. For example, the deprotection of intermediate 18 can be accomplished with acetyl chloride in an art recognized solvent such as methanol.

When piperazine and not piperazinones are desired (i.e. when R¹, R², and R³, are all hydrogen), intermediate 18 can be reduced to afford the desired piperazine prior to the final deprotection step. Such reductions are known to those of skill in the art and include the use of hydride reducing agents, such as lithium aluminum hydride, or other suitable reducing agents in an appropriate solvent such as tetrahydrofuran.

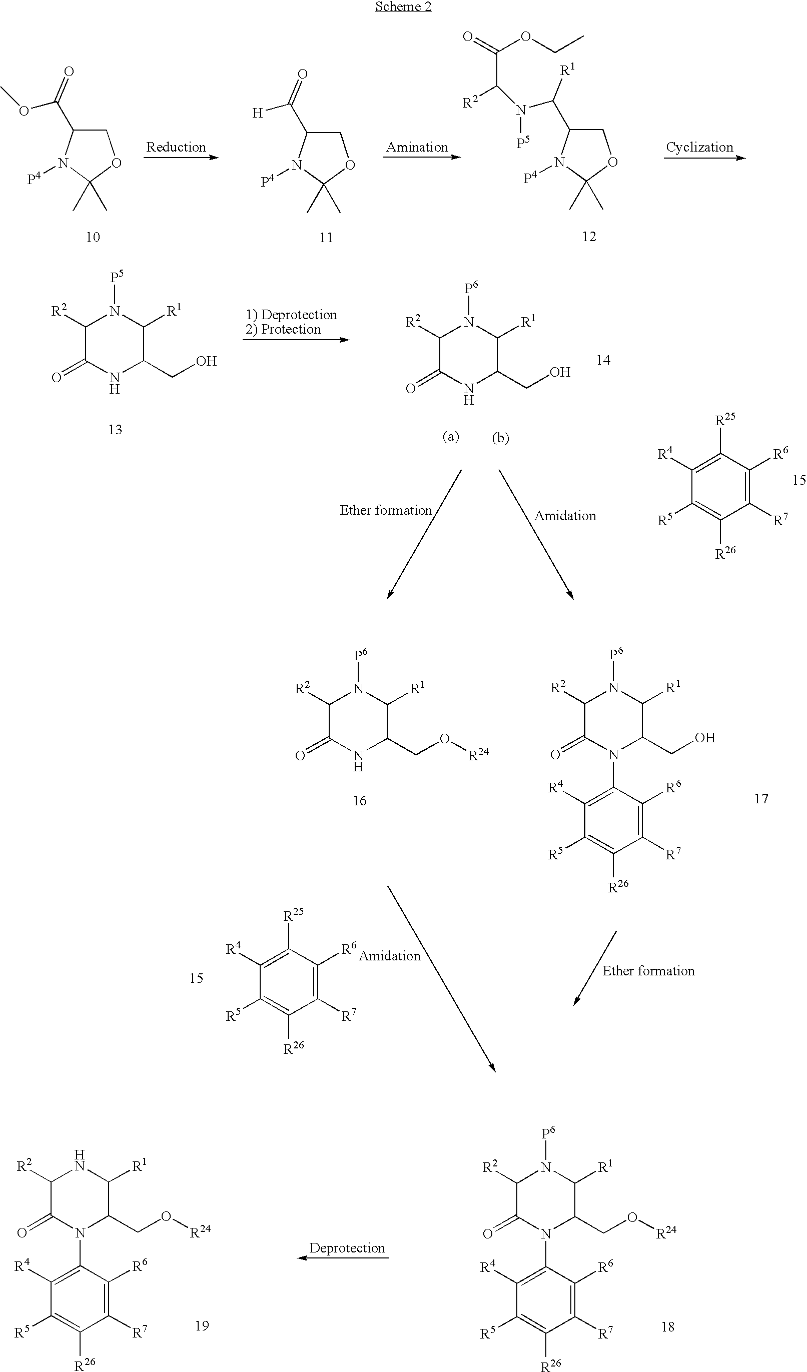

Structures encompassed by Formula I can also be prepared as described in Scheme 3. For example, compounds of Formula I that can be prepared as described in Scheme 3 include those that include an amide, urea or sulfonamide moiety. Such preparations start with the compound 7 prepared as previously described. Alternatively such preparations can start with the compound 17 as previously described. In step (i), the compound 7, or 17, is converted to the triflate intermediate 21. Conversion to the triflate can be accomplished by contacting 7 or 17 with trifluoromethanesulfonic anhydride in, for example, in an art recognized solvent such as dichloromethane, for example. In step (ii) the triflate intermediate 21 is then contacted with an azide such as sodium azide in an art recognized solvent such as N,N-dimethylformamide to afford the azido intermediate 22. The azido intermediate 22 is then reduced to the corresponding amine 23 in step (iii). Such reductions are known to those of skill in the art and include the use of Raney nickel, for example, in an appropriate solvent such as, tetrahydrofuran, for example. The amine intermediate 23 can then be converted to a variety of different compounds with amido, uredo or sulfonamido moieties depending on the electrophile employed. For example, in step (iv), the amine intermediate 23 can be contacted with an appropriate acid chloride in an art recognized solvent, such as dichloromethane for example, to afford the amide 24. Suitable acid chlorides include 2-naphthoyl chloride, benzoyl chloride, 4-chlorobenzoyl chloride, 3,4-dichlorobenzoyl chloride, 2-fluorobenzoyl chloride, 4-fluorobenzoyl chloride, 4-methylbenzoyl chloride, 2-methoxybenzoyl chloride, and 4-methoxybenzoyl chloride, for example. In another example, in step (v), the amine intermediate 23 can be contacted with an appropriate isocyanate in an art recognized solvent, such as dichloromethane for example, to afford the urea 25. Suitable isocyanates include 2-naphthylisocyanate, phenylisocyanate, 4-chloro-phenylisocyanate, 3,4-dichlorophenylisocyanate, 2-fluorophenylisocyanate, 4-fluorophenylisocyanate, 4-methylphenylisocyanate, 2-methoxyphenylisocyanate, and 4-methoxyphenylisocyanate, for example. In another example, in step (vi), the amine intermediate 23 can be contacted with an appropriate sulfonyl chloride in an art recognized solvent, such as dichloromethane for example, to afford the sulfonamide 26. Suitable sulfonyl chlorides include 2-naphthylsulfonyl chloride, phenylsulfonyl chloride, 4-chlorophenylsulfonyl chloride, 3,4-dichlorophenylsulfonyl chloride, 2-fluorophenylsulfonyl chloride, 4-fluorophenylsulfonyl chloride, 4-methylphenylsulfonyl chloride, 2-methoxyphenylsulfonyl chloride, and 4-methoxyphenylsulfonyl chloride, for example. The intermediates 24, 25, and 26 can then be deprotected to afford the final product corresponding to compounds of Formula I. Deprotection of intermediates 24, 25, and 26 can be accomplished using deprotection methods recognized in the art. For example, the deprotection of intermediates 24, 25, and 26 can be accomplished with acetyl chloride in an art recognized solvent such as methanol.

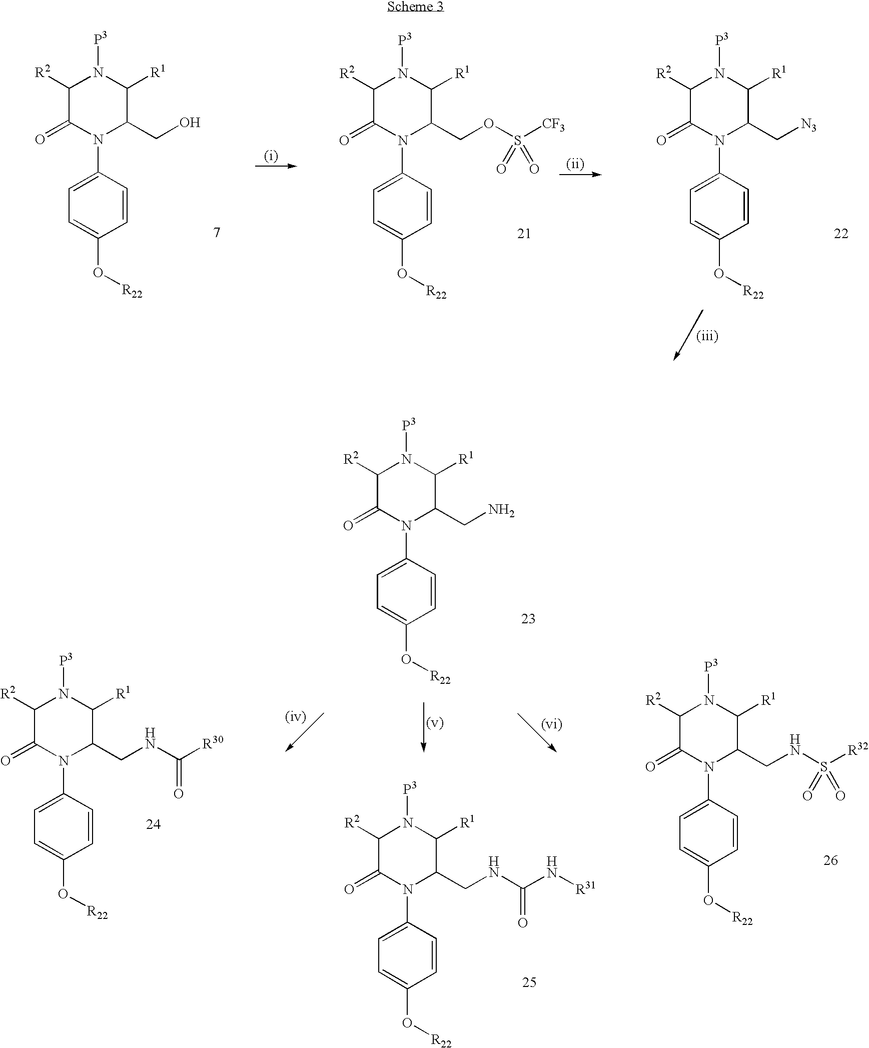

Other methods for making compounds of the invention or variations of the above Schemes are provided in the Examples section.

Not all compounds of the invention falling into a given class may be compatible with some of the reaction conditions described. Such restrictions are readily apparent to those skilled in the art of organic synthesis, and alternative methods must then be used.

Some of the compounds of Formulae I and II are capable of further forming pharmaceutically acceptable acid-addition and/or base salts. All of these forms are within the scope of the present invention. Thus, pharmaceutically acceptable acid addition salts of the compounds of Formulae I and II include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

In some situations, compounds of the invention may exist in isomeric form; for example, as tautomers, enantiomers, or diasteromers. Some compounds may exhibit polymorphism. All tautomers, enantiomers, and diasteromers are incorporated within the definition of the compounds of the invention. It is further to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

The compounds of Formulae I and II can be formulated as pharmaceutical compositions and administered to a mammalian host, such as a human patient in a variety of forms adapted to the chosen route of administration, i.e., orally or parenterally, by intravenous, intramuscular, or subcutaneous routes. Such pharmaceutical compositions can include a compound of Formula I and a pharmaceutically acceptable carrier and/or adjuvant.

The pharmaceutical compositions may comprise in addition one or more compounds useful for treating or preventing hypertension, congestive heart failure, and hyperaldosteronism. Examples for these additional compounds are angiotensin converting enzyme-inhibitors) ramipril, delapril, fosinopril, indolapril, moexipril, perindopril, pivopril, such as captopril, lisinopril, enalapril, quinapril, cilazapril; angiotensin-(I)-receptor antagonists, such as losartan, valsartan, candesartan, candesartan cilexetil, eprosartan, saprisartan, telmisartan, and irbesartan; diuretics, such as hydrochlorothiazide, chlorothiazide, acetazolamide, amiloride, bumetanide, benzthiazide, ethacrynic acid, furosemide, indacrinone, metolazone, spironolactone, triamterene, ticrynafen, chlorthalidone and mefruside; vasodilators, such as hydralazine, minoxidil, diazoxide, and nitroprusside; calcium channel blockers (antagonists), such as amrinone, bencyclane, diltiazem, fendiline, flunarizine, nicardipine, nimodipine, perhexilene, teludipine, verapamil, amlodipine besylate, amlodipine maleate, and gallopamil; nitrates, such as glyceroltrinitrates (nitroglycerin) and isosorbid-dinitrates; beta-receptor blockers, such as atenolol, bisoprolol, metopropol, nadolol, nebivolol, propranolol, timolol, labetalol, betaxolol, carteolol and dilevalol; and alpha-1 adrenoceptor antagonists, such as alfuzosin, doxazosin, prazosin and terazosin; and reserpin.

The pharmaceutical compositions may also comprise in addition one or more agents for reducing the risk of a cardiovascular disorder including anti-inflammatory agents, such as alclofenac, algestone acetonide, alpha arnylase, amcinafal, amcinafide, amfenac sodium, amiprilose hydrochloride, anakinra, anirolac, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cortodoxone, deflazacort, desonide, desoximetasone, dexamethasone dipropionate, diclofenac potassium, diclofenac sodium, diflumidone sodium, diflunisal, difluprednate, diftalone, drocinonide, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, ibufenac, ibuprofen, ibuprofen aluminum, ilonidap, indomethacin, indomethacin sodium, indoprofen, indoxole, intrazole, isoflupredone acetate, isoxepac, isoxicam, ketoprofen, lofemizole hydrochloride, lornoxicam, meclofenamate sodium, meclofenamic acid, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salsalate, salycilates, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tolmetin, tolmetin sodium, triclonide, triflumidate, zidometacin, zomepirac sodium; anti-thrombotic and/or fibrinolytic agents, such as plasminogen (to plasmin via interactions of prekallikrein, kininogens, Factors XII, XIIIa, plasminogen proactivator, and tissue plasminogen activator[TPA]) streptokinase, urokinase: anisoylated plasminogen-streptokinase activator complex; pro-urokinase, (Pro-UK); rTPA (alteplase or activase; r denotes recombinant), rPro-UK, abbokinase, eminase, sreptase anagrelide hydrochloride, bivalirudin, dalteparin sodium, danaparoid sodium, dazoxiben hydrochloride, efegatran sulfate, enoxaparin sodium, ifetroban, ifetroban sodium, tinzaparin sodium, retaplase, trifenagrel, warfarin, dextrans; anti-platelet agents, such as clopridogrel, sulfinpyrazone, aspirin; dipyridamole, clofibrate, pyridinol carbamate, PGE, glucagon, antiserotonin drugs, caffeine, theophyllin pentoxifyllin, ticlopidine, anagrelide; lipid reducing agents, such as gemfibrozil, cholystyramine, colestipol, nicotinic acid, probucol lovastatin, fluvastatin, simvastatin, atorvastatin, pravastatin, cirivastatin; and direct thrombin inhibitors, such as hirudin, hirugen, hirulog, agatroban, PPACK, and thrombin aptamers.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Generally, the concentration in a semi-solid or solid composition such as a gel or a powder will be about 0.1-5 wt-%, preferably about 0.5-2.5 wt-%.

Useful dosages of the compounds of Formulae I and II can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art.

Ideally, the active ingredient should be administered to achieve peak plasma concentrations of the active compound of from about 0.5 to about 75 μM, preferably, about 1 to 50 μM, most preferably, about 0.1 to about 5 μM. This may be achieved, for example, by the intravenous injection of a 0.05 to 5% solution of the active ingredient, optionally in saline, or orally administered as a bolus containing about 10-500 mg of the active ingredient. Desirable blood levels may be maintained by multiple oral dosing, or continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following examples illustrate the various embodiments of the present invention. Those skilled in the art will recognize many variations that are within the spirit of the present invention and scope of the claims.

### Biological Assays

The ability of a compound of the present invention to inhibit rennin is demonstrated using pharmacological models that are well known to the art, for example, using models such as the tests described below.

Determination of Renin IC₅₀ by tGFP FRET Assay

The tGFP FRET (fluorescence resonance energy transfer) assay utilizes a tandem GFP substrate (60 kDa, Discovery Technologies) containing nine amino acid recognition sequences for human renin flanked by two GFP proteins. The assay is used to determine the ability of a compound to act as an inhibitor of renin enzymatic activity by determination of that concentration of test compound that inhibits by 50% (IC₅₀) the ability of renin to cleave the tandem GFP substrate. The IC₅₀ values are determined over an 11-point curve at concentrations of 100 μM to 1 pM. Each compound concentration used to construct the curve was dependent on renin inhibitor potency. For example, subnanomolar IC₅₀ values were determined over an 11-point curve at concentrations of 10 μM to 1 pM. All other IC₅₀ values were determined over an 11-point curve at concentrations of 100 μM to 0.0065 μM. The concentrations were achieved by diluting a 9.1 nM stock of Human recombinant renin in the appropriate amount of buffer containing 50 mM HEPES, 1 mM EDTA, 1% PEG (8000 MW), 1 mM DTT, 0.1% BSA, pH 7.4 to achieve the final concentration of 50.4 μIU. The tGFP substrate stock solution of 43 μM was diluted with the appropriate amount of the above buffer to obtain the final concentration of 650 nM. In addition, 1 μl of the compound is diluted in dimethylsulfoxide (DMSO) to represent an eight-point log scale (5% final). The SR renin and compound are added to a 384 capacity plate by an automated robot (BIOMEK). The plate is incubated for 60 minutes; upon completion the tGFP substrate is added.

The IC50 is determined by monitoring the increase in absorbance at 432/432 nm excitation, 530/475 nm emission with a cutoff at 515/455 nm, in a fluorometric plate reader. The results of this evaluation are shown in Table 1.

TABLE 1

| Compound Name | IC₅₀ μM |
|---|---|
| (6S)-1-{4-[3-(2-methoxybenzyloxy)-propoxy]- | 0.066 |
| phenyl}-6-(naphthalen-2-yloxymethyl)- | |
| piperazin-2-one | |
| (6R)-6-(3,4-dichlorobenzyloxymethyl)-1-{4-[3- | 0.181 |
| (2-methoxybenzyloxy)-propoxy]-phenyl}- | |
| piperazin-2-one | |
| (6R)-6-(2-fluorobenzyloxymethyl)-1-{4-[3-(2- | 0.225 |
| methoxybenzyloxy)-propoxy]-phenyl}- | |
| piperazin-2-one | |
| (6R)-6-(3,4-difluorobenzyloxymethyl)-1-{4-[3- | 0.246 |
| (2-methoxybenzyloxy)-propoxy]-phenyl}- | |
| piperazin-2-one | |
| (6R)-6-(4-chlorobenzyloxymethyl)-1-{4-[3-(2- | 0.295 |
| methoxybenzyloxy)-propoxy]-phenyl}- | |
| piperazin-2-one | |
| (6R)-6-(3-chlorobenzyloxymethyl)-1-{4-[3-(2- | 0.325 |
| methoxybenzyloxy)-propoxy]-phenyl}- | |
| piperazin-2-one | |
| (6R)-1-{4-[3-(2-methoxybenzyloxy)-propoxy]- | 0.337 |
| phenyl}-6-(4-methylbenzyloxymethyl)- | |
| piperazin-2-one | |
| (6R)-6-(4-fluorobenzyloxymethyl)-1-{4-[3-(2- | 0.454 |
| methoxybenzyloxy)-propoxy]-phenyl}- | |
| piperazin-2-one | |
| (6R)-6-(3-methoxybenzyloxymethyl)-1-{4-[3-(2- | 0.612 |
| methoxybenzyloxy)-propoxy]-phenyl}- | |
| piperazin-2-one | |
| (6R)-6-(2-methoxybenzyloxymethyl)-1-{4-[3-(2- | 0.880 |
| methoxybenzyloxy)-propoxy]-phenyl}- | |
| piperazin-2-one | |
| (6R)-6-(3,5-difluorobenzyloxymethyl)-1-{4-[3- | 1.573 |
| (2-methoxybenzyloxy)-propoxy]-phenyl}- | |
| piperazin-2-one | |
| (6R)-6-(4-methoxybenzyloxymethyl)-1-{4-[3-(2- | 3.864 |
| methoxybenzyloxy)-propoxy]-phenyl}- | |
| piperazin-2-one | |
| (6R)-1-{4-[3-(2-methoxybenzyloxy)-propoxy]- | 0.068 |
| phenyl}-6-(4-trifluoromethylbenzyloxymethyl)- | |
| piperazin-2-one | |
| (6R)-6-(2-chlorobenzyloxymethyl)-1-{4-[3-(2- | 0.223 |
| methoxybenzyloxy)-propoxy]-phenyl}- | |
| piperazin-2-one | |
| (6R)-1-{4-[3-(2-methoxybenzyloxy)-propoxy]- | 0.372 |
| phenyl}-6-(3-methylbenzyloxymethyl)- | |
| piperazin-2-one | |
| (6R)-6-(2,6-difluorobenzyloxymethyl)-1-{4-[3- | >1 |
| (2-methoxybenzyloxy)-propoxy]-phenyl}- | |
| piperazin-2-one | |
| (6R)-6-(2,6-dichlorobenzyloxymethyl)-1-{4-[3- | >1 |
| (2-methoxybenzyloxy)-propoxy]-phenyl}- | |
| piperazin-2-one | |
| (6R)-6-(3-fluorobenzyloxymethyl)-1-{4-[3-(2- | >1 |
| methoxybenzyloxy)-propoxy]-phenyl}- | |
| piperazin-2-one | |
| (6R)-6-(4-fluoro-2- | >1 |
| trifluoromethylbenzyloxymethyl)-1-{4-[3-(2- | |
| methoxybenzyloxy)-propoxy]-phenyl}- | |
| piperazin-2-one | |
| (6R)-6-(3,5-dichlorobenzyloxymethyl)-1-{4-[3- | >1 |
| (2-methoxybenzyloxy)-propoxy]-phenyl}- | |
| piperazin-2-one | |
| (6R)-1-{4-[3-(2-methoxybenzyloxy)-propoxy]- | >1 |
| phenyl}-6-(2-methylbenzyloxymethyl)- | |
| piperazin-2-one | |
| (6R)-6-(2-chloro-4-fluorobenzyloxymethyl)-1- | >1 |
| {4-[3-(2-methoxybenzyloxy)-propoxy]-phenyl}- | |
| piperazin-2-one | |
| (6R)-1-{4-[3-(2-methoxybenzyloxy)-propoxy]- | 0.117 |
| phenyl}-6-(pyridin-3-ylmethoxymethyl)- | |
| piperazin-2-one | |
| (6R)-6-(4-chloro-3- | 0.107 |
| trifluoromethylbenzyloxymethyl)-1-{4-[3-(2- | |
| methoxybenzyloxy)-propoxy]-phenyl}- | |
| piperazin-2-one | |
| (6R)-1-{4-[3-(2-methoxybenzyloxy)-propoxy]- | >1 |
| phenyl}-6-(pyridin-4-ylmethoxymethyl)- | |
| piperazin-2-one | |
| (6R)-6-(4-fluoro-3- | 0.132 |
| trifluoromethylbenzyloxymethyl)-1-{4-[3-(2- | |
| methoxybenzyloxy)-propoxy]-phenyl}- | |
| piperazin-2-one | |
| (6R)-6-(4-fluoro-3-methylbenzyloxymethyl)-1- | >1 |
| {4-[3-(2-methoxybenzyloxy)-propoxy]-phenyl}- | |
| piperazin-2-one | |
| (6R)-4-(1-{4-[3-(2-methoxybenzyloxy)- | >1 |
| propoxy]-phenyl}-6-oxopiperazin-2- | |
| ylmethoxymethyl)-benzonitrile | |
| (6R)-1-{4-[3-(2-methoxybenzyloxy)-propoxy]- | >1 |
| phenyl}-6-(pyridin-2-ylmethoxymethyl)- | |
| piperazin-2-one | |
| (6R)-6-(4-bromobenzyloxymethyl)-1-{4-[3-(2- | >1 |
| methoxybenzyloxy)-propoxy]-phenyl}- | |
| piperazin-2-one | |
| acetic acid 2-[7-(1-{4-[3-(2-methoxybenzyloxy)- | 0.00042 |
| propoxy]-phenyl}-6-oxopiperazin-2(R)- | |
| ylmethoxy)-3,4-dihydro-2H-quinolin-1-yl]-ethyl | |
| ester | |
| (2R)-1-{4-[3-(2-methoxybenzyloxy)-propoxy]- | 0.153 |
| phenyl}-2-(naphthalen-2-yloxymethyl)- | |
| piperazine | |
| (2R)-2-(4-methoxybenzyloxymethyl)-1-{4-[3-(2- | 3.990 |
| methoxy-benzyloxy)-propoxy]-phenyl}- | |
| piperazine | |
| (2R)-1-[4-(3-benzyloxypropoxy)-phenyl]-2-(4- | 10.900 |
| methoxybenzyloxymethyl)-piperazine | |
| (2R)-1-(4-benzyloxyphenyl)-2-(naphthalen-2- | >1 |
| yloxymethyl)-piperazine | |
| (2R)-1-(4-benzyloxyphenyl)-2-(4- | >1 |
| methoxybenzyloxymethyl)-piperazine | |
| (6R)-1-{4-[3-(2-methoxybenzyloxy)-propoxy]- | .054 |
| phenyl}-6-(naphthalen-2-yloxymethyl)- | |
| piperazin-2-one | |
| (2R)-1-[4-(3-benzyloxypropoxy)-phenyl]-2- | 1.420 |
| (naphthalen-2-yloxymethyl)-piperazine | |
| (6R)-1-{3-fluoro-4-[3-(2-methoxybenzyloxy)- | 0.152 |
| propoxy]-phenyl}-6-(naphthalen-2- | |
| yloxymethyl)-piperazin-2-one | |
| (2R)-1-{4-[3-(2-methoxybenzyloxy)-propoxy]- | 0.440 |
| phenyl}-2-(5,6,7,8-tetrahydronaphthalen-2- | |
| yloxymethyl)-piperazine | |
| (6R)-1-{4-[3-(2-methoxybenzyloxy)-propoxy]- | 0.822 |
| phenyl}-6-(quinolin-7-yloxymethyl)-piperazin-2- | |
| one | |
| (6R)-1-{4-[3-(2-methoxybenzyloxy)-propoxy]- | 0.124 |
| phenyl}-6-(1,2,3,4-tetrahydroquinolin-7- | |
| yloxymethyl)-piperazin-2-one | |
| (6R)-1-{3,5-difluoro-4-[3-(2- | 0.883 |
| methoxybenzyloxy)-propoxy]-phenyl}-6- | |
| (naphthalen-2-yloxymethyl)-piperazin-2-one | |
| (6R)-6-[1-(3-hydroxypropyl)-1,2,3,4- | 0.037 |
| tetrahydroquinolin-7-yloxymethyl]-1-{4-[3-(2- | |
| methoxybenzyloxy)-propoxy]-phenyl}- | |
| piperazin-2-one | |
| (6R)-6-benzyloxymethyl-1-{4-[3-(2- | 7.014 |
| methoxybenzyloxy)-propoxy]-phenyl}- | |
| piperazin-2-one | |
| (6S)-6-(4-fluorobenzyloxymethyl)-1-{4-[3-(2- | 5.586 |
| methoxybenzyloxy)-propoxy]-phenyl}- | |
| piperazin-2-one | |
| 4-[(2R)-2-(naphthalen-2-yloxymethyl)-6- | >1 |
| oxopiperazin-1-yl]-N-phenethylbenzamide | |
| (6R)-1-[4-methoxy-3-(3-methoxypropoxy)- | 3.841 |
| phenyl]-6-(naphthalen-2-yloxymethyl)-piperazin- | |
| 2-one | |
| N-(2-ethoxyethyl)-4-[(2R)-2-(naphthalen-2- | >1 |
| yloxymethyl)-6-oxopiperazin-1-yl]-benzamide | |
| N-[2-(3-methoxyphenyl)-ethyl]-4-[(2R)-2- | >1 |
| (naphthalen-2-yloxymethyl)-6-oxopiperazin-1- | |
| yl]-benzamide | |
| (6R)-6-(isoquinolin-7-yloxymethyl)-1-{4-[3-(2- | 0.528 |
| methoxybenzyloxy)-propoxy]-phenyl}- | |
| piperazin-2-one | |
| (6R)-1-{4-[3-(2-methoxybenzyloxy)-propoxy]- | 3.450 |
| phenyl}-6-(quinolin-6-yloxymethyl)-piperazin-2- | |
| one | |
| 4-[(2R)-2-(naphthalen-2-yloxymethyl)-6- | >1 |
| oxopiperazin-1-yl]-N-(2-phenoxyethyl)- | |
| benzamide | |
| (6R)-6-(1-acetyl-1,2,3,4-tetrahydroquinolin-6- | >1 |
| yloxymethyl)-1-{4-[3-(2-methoxybenzyloxy)- | |
| propoxy]-phenyl}-piperazin-2-one | |
| (6R)-1-{4-[3-(2-methoxybenzyloxy)-propoxy]- | 0.379 |
| phenyl}-6-(1-thiazol-4-ylmethyl-1,2,3,4- | |
| tetrahydroquinolin-7-yloxymethyl)-piperazin-2- | |
| one | |
| 2-[7-(1-{4-[3-(2-methoxybenzyloxy)-propoxy]- | 0.435 |
| phenyl}-6-oxopiperazin-2R-ylmethoxy)-3,4- | |
| dihydro-2H-quinolin-1-yl]-acetamide | |
| (6R)-6-[1-(2-hydroxyethyl)-1,2,3,4- | 0.064 |
| tetrahydroquinolin-7-yloxymethyl]-1-{4-[3-(2- | |
| methoxybenzyloxy)-propoxy]-phenyl}- | |
| piperazin-2-one | |
| naphthalene-2-carboxylic acid (2R)-1-{4-[3-(2- | >1 |
| methoxy-benzyloxy)-propoxy]-phenyl}-6-oxo- | |
| piperazin-2-yl methyl ester | |
| 4-methyl-benzoic acid (2R)-1-{4-[3-(2-methoxy- | >1 |
| benzyloxy)-propoxy]-phenyl}-6-oxo-piperazin- | |
| 2-yl methyl ester | |
| 4-chloro-benzoic acid (2R)-1-{4-[3-(2-methoxy- | >1 |
| benzyloxy)-propoxy]-phenyl}-6-oxo-piperazin- | |
| 2-yl methyl ester | |
| benzoic acid (2R)-1-{4-[3-(2- | >1 |
| methoxybenzyloxy)-propoxy]-phenyl}-6- | |
| oxopiperazin-2-yl methyl ester | |
| (2R)-1-{4-[3-(4-chlorobenzyloxy)-propoxy]- | 43.700 |
| phenyl}-2-(4-methoxybenzyloxymethyl)- | |
| piperazine | |
| (2R)-1-{4-[3-(3,4-dichlorobenzyloxy)-propoxy]- | 11.500 |
| phenyl}-2-(4-methoxybenzyloxymethyl)- | |
| piperazine; | |
| (2R)-1-{4-[3-(3-chlorobenzyloxy)-propoxy]- | 5.040 |
| phenyl}-2-(4-methoxybenzyloxymethyl)- | |
| piperazine | |
| (2R)-2-(4-methoxybenzyloxymethyl)-1-{4-[3-(4- | >1 |
| methoxybenzyloxy)-propoxy]-phenyl}- | |
| piperazine; | |
| (2R)-1-{4-[3-(2-chlorobenzyloxy)-propoxy]- | 15.900 |
| phenyl}-2-(4-methoxybenzyloxymethyl)- | |
| piperazine; | |
| (2R)-1-{4-[3-(3,5-difluorobenzyloxy)-propoxy]- | 4.340 |
| phenyl}-2-(4-methoxybenzyloxymethyl)- | |
| piperazine | |
| (2R)-2-(4-methoxybenzyloxymethyl)-1-{4-[3-(4- | 60.200 |
| methylbenzyloxy)-propoxy]-phenyl}-piperazine | |
| (2R)-2-(4-methoxybenzyloxymethyl)-1-{4-[3-(3- | 15.600 |
| methoxybenzyloxy)-propoxy]-phenyl}- | |
| piperazine | |
| (2R)-2-(4-methoxybenzyloxymethyl)-1-{4-[3-(2- | >1 |
| methoxyphenoxy)-propoxymethyl]-phenyl}- | |
| piperazine | |
| (6R)-6-(4-fluorobenzyloxymethyl)-1-{4-[4-(2- | 3.840 |
| methoxyphenoxy)-butoxy]-phenyl}-piperazin-2- | |
| one | |
| (2R)-1-{4-[2-(2-methoxybenzyloxy)- | 30.500 |
| ethoxymethyl]-phenyl}-2-(4- | |
| methoxybenzyloxymethyl)-piperazine | |
| (6R)-1-{4-[4-(2-methoxyphenoxy)-butoxy]- | 1.729 |
| phenyl}-6-(naphthalen-2-yloxymethyl)- | |
| piperazin-2-one | |
| (6R)-6-(4-fluorobenzyloxymethyl)-1-{4-[2-(2- | 4.090 |
| methoxybenzyloxy)-ethoxymethyl]-phenyl}- | |
| piperazin-2-one | |
| (2R)-2-(4-methoxybenzyloxymethyl)-1-{4-[4-(2- | 42.000 |
| methoxyphenoxy)-butoxy]-phenyl}-piperazine | |
| (6R)-6-(4-fluorobenzyloxymethyl)-1-{4-[3-(2- | >1 |
| methoxyphenoxy)-propoxymethyl]-phenyl}- | |
| piperazin-2-one | |
| (6R)-1-{4-[3-(2-methoxybenzyloxy)-propoxy]- | 13.100 |
| phenyl}-6-(quinolin-2-yloxymethyl)-piperazin-2- | |
| one | |
| (6R)-1-{4-[3-(2-methoxybenzyloxy)-propoxy]- | 1.000 |
| phenyl}-6-(quinoxalin-2-yloxymethyl)- | |
| piperazin-2-one | |
| (6R)-1-{4-[3-(2-methoxybenzyloxy)-propoxy]- | 76.375 |
| phenyl}-6-(pyrazin-2-yloxymethyl)-piperazin-2- | |
| one | |
| (6R)-1-{4-[3-(2-methoxybenzyloxy)-propoxy]- | 20.933 |
| phenyl}-6-(pyridin-2-yloxymethyl)-piperazin-2- | |
| one | |
| (6R)-1-{4-[3-(2-methoxybenzyloxy)-propoxy]- | 33.857 |
| phenyl}-6-(pyrimidin-2-yloxymethyl)-piperazin- | |
| 2-one | |
| 2-methoxy-N-([2R]-1-{4-[3-(2-methoxy- | >1 |
| benzyloxy)-propoxy]-phenyl}-6-oxo-piperazin- | |
| 2-ylmethyl)-benzamide | |
| 4-chloro-N-([2R]-1-{4-[3-(2-methoxy- | >1 |
| benzyloxy)-propoxy]-phenyl}-6-oxo-piperazin- | |
| 2-ylmethyl)-benzamide | |
| N-([2R]-1-{4-[3-(2-methoxy-benzyloxy)- | >1 |
| propoxy]-phenyl}-6-oxo-piperazin-2-ylmethyl)- | |
| benzamide | |
| naphthalene-2-carboxylic acid ([2R]-1-{4-[3-(2- | >1 |
| methoxy-benzyloxy)-propoxy]-phenyl}-6-oxo- | |
| piperazin-2-ylmethyl)-amide | |
| 2-fluoro-N-([2R]-1-{4-[3-(2-methoxy- | >1 |
| benzyloxy)-propoxy]-phenyl}-6-oxo-piperazin- | |
| 2-ylmethyl)-benzamide | |
| (2R)-1-{4-[3-(2-fluorobenzyloxy)-propoxy]- | 0.597 |
| phenyl}-2-(naphthalen-2-yloxymethyl)- | |
| piperazine | |
| (2R)-1-{4-[3-(2-ethoxybenzyloxy)-propoxy]- | 1.980 |
| phenyl}-2-(naphthalen-2-yloxymethyl)- | |
| piperazine | |
| (2R)-1-{4-[3-(3-methoxybenzyloxy)-propoxy]- | 23.700 |
| phenyl}-2-(naphthalen-2-yloxymethyl)- | |
| piperazine | |
| (2R)-1-{4-[3-(2-methoxybenzyloxy)-propoxy]- | 1.700 |
| phenyl}-2-(naphthalen-2-ylmethoxymethyl)- | |
| piperazine | |
| (2R)-2-(biphenyl-3-ylmethoxymethyl)-1-{4-[3- | 1.020 |
| (2-methoxybenzyloxy)-propoxy]-phenyl}- | |
| piperazine | |
| (6R)-6-(biphenyl-4-yloxymethyl)-1-(4-[3-(2- | 2.519 |
| methoxybenzyloxy)-propoxy]-phenyl)-piperazin- | |
| 2-one | |
| N-[4-([2R]-1-{4-[3-(2-methoxy-benzyloxy)- | >1 |
| propoxy]-phenyl}-6-oxo-piperazin-2- | |
| ylmethoxy)-phenyl]-acetamide; | |
| (2R)-1-{4-[2-(2-methoxybenzyloxy)- | 2.549 |
| ethoxymethyl]-phenyl}-2-(naphthalene-2- | |
| yloxymethyl)-piperazine | |
| 4-([2R]-1-{4-[3-(2-methoxybenzyloxy)- | 4.050 |
| propoxy]-phenyl}-6-oxopiperazin-2-ylmethoxy)- | |
| N,N-dimethyl-benzamide | |
| 2-[(5R)-3-(2-methoxybenzyloxy)-propoxy]-5-[2- | >1 |
| (naphthalene-2-yloxymethyl)-6-oxopiperazin-1- | |
| yl]-benzoic acid methyl ester | |
| (2R)-1-(4-[3-(2-methoxyphenoxy)- | 26.255 |
| propoxymethyl]-phenyl)-2-(naphthalene-2- | |
| yloxymethyl)-piperazine | |
| 2-[(5R)-3-(2-methoxybenzyloxy)-propoxy]-5-[2- | >1 |
| (naphthalene-2-yloxymethyl)-6-oxopiperazin-1- | |
| yl]-benzoic acid | |
| (2R)-1-(4-methoxymethylphenyl)-2- | >1 |
| (naphthalene-2-yloxymethyl)-piperazine; | |
| (6R)-1-(3-chloro-4-[3-(2-methoxybenzyloxy)- | >1 |
| propoxy]-phenyl)-6-(naphthalene-2- | |
| yloxymethyl)-piperazin-2-one | |
| (6R)-1-{4-[3-(2-methoxybenzylsulfanyl)- | >1 |
| propoxy]-phenyl}-6-(naphthalene-2- | |
| yloxymethyl)-piperazin-2-one | |
| (2R)-1-{4-[3-(2-methoxybenzylsulfanyl)- | >1 |
| propoxy]-phenyl}-2-(naphthalene-2- | |
| yloxymethyl)-piperazine | |
| (6R)-1-{4-[3-(2-methoxybenzyloxy)-propoxy]-3- | 0.167 |
| methyl-phenyl}-6-(naphthalene-2-yloxymethyl)- | |
| piperazin-2-one | |
| (2R)-1-{4-{2-[2-(2-methoxyphenyl)-ethoxy]- | 1.070 |
| ethoxy}-ethoxy}-phenyl)-2-(naphthalene-2- | |
| yloxymethyl)-piperazine | |
| (6R)-1-{4-[3-(2-methoxybenzyloxy)-propoxy]- | 0.225 |
| phenyl}-6-(7-methoxynaphthalen-2- | |
| yloxymethyl)-piperazin-2-one | |
| (6R)-6-(biphenyl-3-yloxymethyl)-1-{4-[3-(2- | 1.513 |
| methoxybenzyloxy)-propoxy]-phenyl}- | |
| piperazin-2-one | |
| (6R)-1-(3-methoxy-4-[3-(2-methoxybenzyloxy)- | 0.537 |
| propoxy]-phenyl)-6-(naphthalene-2- | |
| yloxymethyl)-piperazin-2-one | |
| (2R)-1-{4-[4-(2-methoxyphenoxy)-butoxy]- | 1.360 |
| phenyl}-2-(naphthalene-2-yloxymethyl)- | |
| piperazine | |
| [6-([2R]-1-{4-[3-(2-methoxybenzyloxy)- | 0.00017 |
| propoxy]-phenyl}-6-oxopiperazin-2- | |
| ylmethylsulfanyl)-3-oxo-2,3- | |
| dihydrobenzo[1,4]oxazin-4-yl]-acetic acid ethyl | |
| ester | |
| 6-([2R]-1-{4-[3-(2-methoxybenzyloxy)- | 0.00018 |
| propoxy]-phenyl}-6-oxopiperazin-2- | |
| ylmethylsulfanyl)-4-(3-methoxypropyl)-4H- | |
| benzo[1,4]oxazin-3-one | |
| N-{2-[7-([2R]-1-{4-[3-(2-methoxybenzyloxy)- | 0.00035 |
| propoxy]-phenyl}-6-oxopiperazin-2-ylmethoxy)- | |
| 3,4-dihydro-2H-quinolin-1-yl]-ethyl}-acetamide | |
| 3-[7-([2R]-1-{4-[3-(2-methoxybenzyloxy)- | 0.00036 |
| propoxy]-phenyl}-6-oxopiperazin-2-ylmethoxy)- | |
| 3,4-dihydro-2H-quinolin-1-yl]-propionic acid | |
| methyl ester | |
| N-{2-[7-([2R]-1-{4-[3-(2-methoxybenzyloxy)- | 0.00053 |
| propoxy]-phenyl}-piperazin-2-ylmethoxy)-3,4- | |
| dihydro-2H-quinolin-1-yl]-ethyl}-acetamide | |
| 3-[5-([2R]-1-{4-[3-(2-methoxybenzyloxy)- | 0.00162 |
| propoxy]-phenyl}-6-oxopiperazin-2-ylmethoxy)- | |
| indol-1-yl]-propionic acid methyl ester | |
| 7-([2S]-1-{4-[3-(2-methoxybenzyloxy)- | 0.00170 |
| propoxy]-phenyl}-6-oxopiperazin-2-ylmethoxy)- | |
| 1-(3-methoxypropyl)-3,4-dihydro-1H-quinolin-2- | |
| one | |
| (6R)-1-{4-[3-(2-methoxybenzyloxy)-propoxy]- | 0.00317 |
| phenyl}-6-[1-(3-methoxypropyl)-1,2,3,4- | |
| tetrahydroquinolin-7-yloxymethyl]-piperazin-2- | |
| one | |
| 7-([2R]-1-{4-[3-(2-methoxybenzyloxy)- | 0.00391 |
| propoxy]-phenyl}-6-oxopiperazin-2-ylmethoxy)- | |
| 1-(3-methoxypropyl)-3,4-dihydro-1H-quinolin-2- | |
| one | |
| [7-([2R]-1-{4-[3-(2-methoxybenzyloxy)- | 0.00400 |
| propoxy]-phenyl}-6-oxopiperazin-2-ylmethoxy)- | |
| 2-oxo-3,4-dihydro-2H-quinolin-1-yl]-acetic acid | |
| methyl ester | |
| [7-([2R]-1-{4-[3-(2-methoxybenzyloxy)- | 0.00409 |
| propoxy]-phenyl}-6-oxopiperazin-2-ylmethoxy)- | |
| 3,4-dihydro-2H-quinolin-1-yl]-acetic acid methyl | |
| ester | |
| N-{2-[5-([2R]-1-{4-[3-(2-methoxybenzyloxy)- | 0.00466 |
| propoxy]-phenyl}-6-oxopiperazin-2-ylmethoxy)- | |
| indol-1-yl]-ethyl}-acetamide | |
| N-{2-[7-([2R]-1-{4-[3-(2-fluorobenzyloxy)- | 0.00500 |
| propoxy]-phenyl}-6-oxopiperazin-2-ylmethoxy)- | |
| 3,4-dihydro-2H-quinolin-1-yl]-ethyl}-acetamide | |
| [6-([2R]-1-{4-[3-(2-methoxybenzyloxy)- | 0.00600 |
| propoxy]-phenyl}-6-oxopiperazin-2-ylmethoxy)- | |
| indol-1-yl]-acetic acid ethyl ester | |
| [5-([2R]-1-{4-[3-(2-methoxybenzyloxy)- | 0.00600 |
| propoxy]-phenyl}-6-oxopiperazin-2-ylmethoxy)- | |
| 2-methyl-indol-1-yl]-acetic acid methyl ester | |
| [6-([2R]-1-{4-[3-(2-methoxybenzyloxy)- | 0.00683 |
| propoxy]-phenyl}-6-oxopiperazin-2-ylmethoxy)- | |
| 3-oxo-2,3-dihydrobenzo[1,4]oxazin-4-yl]-acetic | |
| acid methyl ester | |
| propionic acid 2-[7-([2R]-1-{4-[3-(2- | 0.00900 |
| methoxybenzyloxy)-propoxy]-phenyl}-6- | |
| oxopiperazin-2-ylmethoxy)-3,4-dihydro-2H- | |
| quinolin-1-yl]-ethyl ester | |
| 3-[6-([2R]-1-{4-[3-(2-methoxybenzyloxy)- | 0.00959 |
| propoxy]-phenyl}-6-oxopiperazin-2-ylmethoxy)- | |
| 2,3-dihydroindol-1-yl]-propionic acid methyl | |
| ester | |
| [5-([2S]-1-{4-[3-(2-methoxybenzyloxy)- | 0.01000 |
| propoxy]-phenyl}-6-oxopiperazin-2-ylmethoxy)- | |
| indol-1-yl]-acetic acid methyl ester | |

The foregoing biological tests establish that the compounds of the present invention are potent inhibitors of renin. Accordingly, the compounds of the present invention are useful in pharmaceutical formulations for preventing and treating disorders in which rennin plays a significant pathological role. Such disorders include hypertension and congestive heart failure, end organ protection, stroke, myocardial infarction, glaucoma and hyperaldosteronism.

To further assist in understanding the present invention, the following non-limiting examples of such renin inhibitory compounds are provided. The following examples, of course, should not be construed as specifically limiting the present invention, variations presently known or later developed, which would be within the purview of one skilled in the art and considered to fall within the scope of the present invention as described herein. Preferred synthetic routes for intermediates involved in the synthesis as well as the resulting renin inhibitory compounds of the present invention follow.

### Preparation Methods

The compounds disclosed in the following Examples may be prepared following the procedures described in the following exemplary methods.

Method A: Preparation of (3R)-3-Hydroxymethyl-4-(4-[3-(2-Methoxybenzyloxy)-propoxy]-phenyl)-5-oxopiperazine-1-carboxylic acid tert-butyl ester (Scheme 4)

(i) 4-Benzyloxyaniline hydrochloride (50 g, 0.21 mol) was dissolved in 750 mL of THF. Solid potassium carbonate (44 g, 0.32 mol) was added and the mixture stirred at room temperature for 15 min. Chloroacetyl chloride (26.35 g, 0.23 mol) was added via syringe at room temperature and the reaction mixture stirred at room temperature for 6 h. Diethyl ether was added (300 mL) and the mixture filtered through a pad of celite. The solution was concentrated under reduced pressure to a brown solid. The solid was washed with hexanes to afford 54.85 g (94%) of N-(4-benzyloxyphenyl)-2-chloroacetamide as a brown solid. MS: m/z 276.1 (M+1).

(ii) N-(4-Benzyloxyphenyl)-2-chloroacetamide (54 g, 0.20 mol) was dissolved in THF (600 mL). Benzylamine (46.17 g, 0.43 mol) was added via syringe and the reaction mixture heated to 98° C. for 18 h. The reaction mixture was cooled to room temperature and filtered. The mother liquor was evaporated under reduced pressure to obtain a brown solid. The solid was washed with copious amounts of diethyl ether to obtain 63 g (93%) of 2-benzylamino-N-(4-benzyloxyphenyl)-acetamide as a beige solid. MS: m/z 375 (M+1).

Alternatively, 2-benzylamino-N-(4-benzyloxyphenyl)-acetamide was prepared from 4-Benzyloxyaniline hydrochloride as demonstrated in (ia) and (iia) below.

(ia) 4-Benzyloxyaniline hydrochloride (4.68 g, 19.8 mmole) was suspended in anhydrous dichloromethane (66 mL). Diisopropylethylamine (8.64 mL, 49.6 mmole), 4-(N,N-dimethylamino)pyridine (240 mg, 2.0 mmole), N,N′-diisopropylcarbodiimide (3.88 mL, 24.8 mmole), and (N-Benzyl-N-tert-butoxycarbonyl)glycine (5.79 g, 21.8 mmole) were added sequentially. The resulting solution was stirred at room temperature for 18 h, diluted with dichloromethane, washed with water, aqueous saturated sodium bicarbonate, aqueous 10% citric acid, and water. The organic layer was dried over anhydrous magnesium sulfate, filtered, and concentrated under reduced pressure. The residue was purified by flash column chromatography (silica gel, gradient: 10% ethyl acetate/hexanes to 30% ethyl acetate/hexanes) to yield 8.23 g (93%) of benzyl-[(4-benzyloxyphenylcarbamoyl)-methyl]-carbamic acid tert-butyl ester as a orange viscous oil. MS: m/z 447.2 (M+1).

(iia) Benzyl-[(4-benzyloxyphenylcarbamoyl)-methyl]-carbamic acid tert-butyl ester (8.20 g, 18.4 mmole) was dissolved in methanol (90 mL). A 6 N hydrochloric acid solution in water (9.2 mL) was added, and the mixture was heated to reflux for 18 h. After cooling to room temperature, the white precipitate was collected and rinsed with ice-cold methanol. The solid was slurried in methanol (200 mL) and carefully neutralized by the addition of aqueous saturated sodium bicarbonate. The aqueous layer was extracted with ethyl acetate (3×). The combined organic layers were washed with water (100 mL), dried over anhydrous magnesium sulfate, filtered, and concentrated to afford 4.32 g (68%) of 2-benzylamino-N-(4-benzyloxyphenyl)-acetamide as a white solid that was used without further purification. MS: m/z 375.2 (M+1).

(iii) 2-Benzylamino-N-(4-benzyloxyphenyl)-acetamide (63 g, 0.18 mol) was dissolved in methanol (1 L). Magnesium sulfate was added (26 g) followed by dichloromethane (0.35 L). S-Epichlorohydrin (42.07 g, 0.45 mol) was added via syringe. Once addition was complete the reaction mixture was heated to 35° C. and stirred for 72 h. The reaction mixture was filtered through a pad of celite. The pad was washed with ethyl acetate and organic fractions combined. The organic layer was concentrated under reduced pressure to yield 79.8 g (100%) of 2-{benzyl-[(2S)-3-chloro-2-hydroxypropyl]-amino}-N-(4-benzyloxyphenyl)-acetamide as a dark oil. MS: m/z 439.1 (M+1).

(iv) 2-{Benzyl-[(2S)-3-chloro-2-hydroxypropyl]-amino}-N-(4-benzyloxyphenyl)-acetamide (79.8 g, 0.18 mol) was dissolved in a 1:1 mixture of methanol and tetrahydrofuran (2 L). A 5% solution of aqueous sodium hydroxide solution (1 L) was added dropwise and the mixture stirred for 18 h at room temperature. The reaction mixture was poured into brine (2 L) and extracted with ethyl acetate (3×1 L). The organic layer was washed with brine (2×500 mL) and dried over anhydrous MgSO₄. The mixture was filtered and the organic layer concentrated under reduced pressure. The resulting beige solid (80 g) was recrystallized from hot ethyl acetate and ethanol to yield 31 g of (6R)-4-benzyl-1-(4-benzyloxyphenyl)-6-hydroxymethylpiperazin-2-one (compound 4). A second crop yielded 15 g. The remaining mixture was purified by flash column chromatography (silica gel, gradient: 50% ethyl acetate/hexanes to 75% ethyl acetate/hexanes) to yield 10 g of product. Total amount of (6R)-4-benzyl-1-(4-benzyloxyphenyl)-6-hydroxymethylpiperazin-2-one isolated was 56 g (77%). MS: m/z 403.1 (M+1).

(v) (6R)-4-Benzyl-1-(4-benzyloxyphenyl)-6-hydroxymethylpiperazin-2-one (17 g, 42 mmol) was dissolved in mixture of methanol and tetrahydrofuran (1:1, 300 mL). Di-tert-butyl carbonate (11.1 g, 50 mmol) was added followed by palladium hydroxide (2 g, 20% on carbon). The reaction mixture was exposed to an atmosphere of hydrogen at 55 psi for 17 h. The reaction mixture was filtered through a pad of celite and the solvent concentrated under reduced pressure. The resultant oil was purified by flash column chromatography (silica gel, gradient: 50% ethyl acetate/hexanes to 100% ethyl acetate) to yield 12.65 g (93%) of (3R)-3-hydroxymethyl-4-(4-hydroxy-phenyl)-5-oxopiperazine-1-carboxylic acid tert-butyl ester as a white solid. MS: m/z 403.1 (M+1).

(vi) (3R)-3-Hydroxymethyl-4-(4-hydroxyphenyl)-5-oxopiperazine-1-carboxylic acid tert-butyl ester (5 g, 15.5 mmol) was dissolved in acetonitrile (30 mL). 1-(3-iodopropoxymethyl)-2-methoxybenzene (5.70 g, 18.6 mmol) and potassium carbonate (3.22 g, 23.3 mmol) were added at room temperature. The reaction mixture was heated to reflux for 18 h. The reaction mixture was cooled to room temperature and water (100 mL) added. The mixture was extracted with ethyl acetate (200 mL). The organic layer was washed with brine (2×100 mL), dried over anhydrous magnesium sulfate, filtered and concentrated under reduced pressure to obtain an oil. The oil was purified by flash column chromatography (silica gel, gradient: 50 ethyl acetate/hexanes to 100% ethyl acetate) to afford 6.94 g (89%) of (3R)-3-hydroxymethyl-4-(4-[3-(2-methoxybenzyloxy)-propoxy]-phenyl)-5-oxopiperazine-1-carboxylic acid tert-butyl ester. MS: m/z 501.3 (M+1).

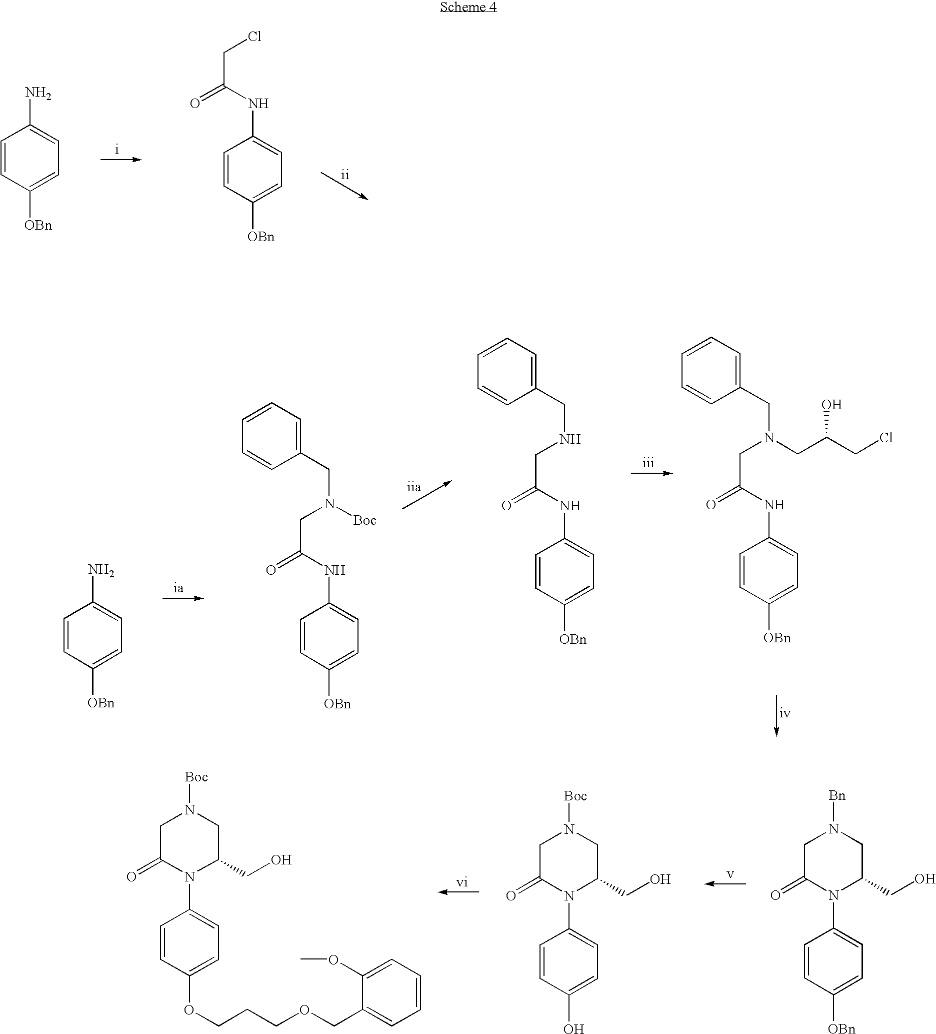

Method B: Formation of Aryl Ethers as Exemplified by 1-{4-[3-(2-Methoxybenzyloxy)-propoxy]-phenyl}-6-(naphthalen-2-yloxymethyl)-piperazin-2-one (Scheme 5)

(i) A N₂-flushed, round bottom flask, was charged with (3R)-3-hydroxymethyl-4-(4-[3-(2-methoxybenzyloxy)-propoxy]-phenyl)-5-oxopiperazine-1-carboxylic acid tert-butyl ester (0.500 g, 0.999 mmol), 2-naphthol (216 mg, 1.498 mmol), and dichloromethane (15 mL). Triphenylphosphine supported on polystyrene (0.975 g, 1.598 mmol, 1.64 mmol/g) was added at room temperature, and the resulting slurry was stirred for 10 minutes. The reaction mixture was cooled in an ice bath, and diisopropyl azodicarboxylate (0.236 ml, 1.199 mmol) was added dropwise over a two minute period. The orange-red reaction mixture was stirred for 18 h, slowly warming to room temperature. The reaction mixture was filtered, and the resin rinsed with dichloromethane (3×). The crude product was purified by flash column chromatography (silica gel, gradient: 20% ethyl acetate/hexanes to 50% ethyl acetatethexanes) to afford 0.507 g (81%) of (3R)-4-(4-[3-(2-methoxybenzyloxy)-propoxy)-phenyl])-3-(naphthalene-2-yloxymethyl)-5-oxopiperazine-1-carboxylic acid tert-butyl ester as a white solid. [α]_{D}=+43.5 (c=2.6, CHCl₃); MS: m/z 627.1 (M+1).

(ii) (3R)-4-(4-[3-(2-Methoxybenzyloxy)-propoxy)-phenyl])-3-(naphthalene-2-yloxymethyl)-5-oxopiperazine-1-carboxylic acid tert-butyl ester (55 mg, 87.7 mmol) was dissolved in methanol (2 mL) under nitrogen and cooled to 0° C. Acetyl chloride (68.9 mg, 0.877 mmol) was added dropwise. The nitrogen inlet needle was removed, and the reaction mixture allowed to warm to room temperature over a period of 18 h. The reaction mixture was quenched with aqueous saturated sodium bicarbonate. The mixture was extracted with ethyl acetate (3×10 mL). The organic layer was dried over anhydrous magnesium sulfate, filtered and concentrated under reduced pressure. The resultant solid was purified by flash column chromatography (silica gel, gradient: 100% dichloromethane then 95/5 dichloromethane/methanol to 90/10 dichloromethane/methanol) to afford 36.1 mg (78.1%) of 1-{4-[3-(2-methoxybenzyloxy)-propoxy]-phenyl}-6-(naphthalen-2-yloxymethyl)-piperazin-2-one as a semi-solid. MS: m/z 527.1 (M+1).

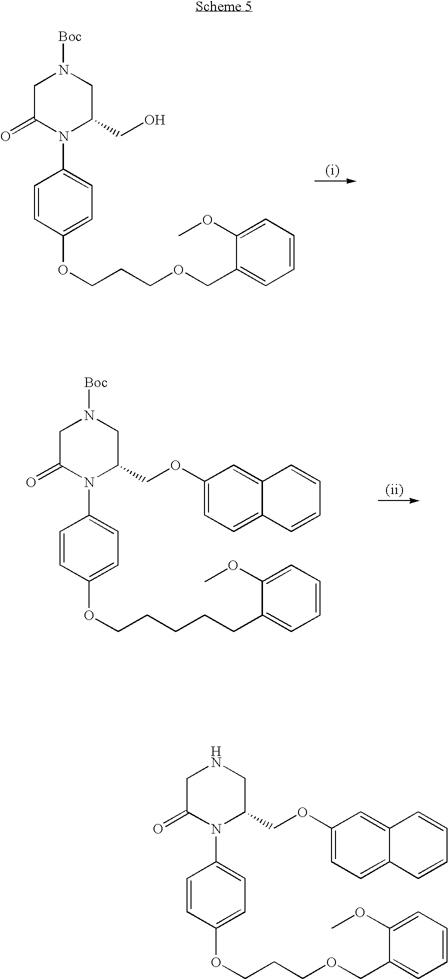

Method C: Formation of Ethers as Exemplified by (6R)-6-benzyloxymethyl-1-{4-[3-(2-methoxybenzyloxy)-propoxy]-phenyl}-piperazin-2-one (Scheme 6)

(i) (3R)-3-Hydroxymethyl-4-(4-[3-(2-methoxybenzyloxy)-propoxy]-phenyl)-5-oxopiperazine-1-carboxylic acid tert-butyl ester (150 mg, 0.300 mmole) was dissolved in anhydrous DMF (2 mL), and cooled in an ice bath. Sodium hydride (9 mg, 0.36 mmole), 95%) was added in a single portion, and the resulting white suspension was stirred at 0° C. for 20 min. 15-Crown-5 (12 μL, 0.06 mmole) and benzyl bromide (53 μL, 0.45 mmole) were added. The ice bath was removed, and the reaction mixture was stirred at room temperature for 1 h. The clear solution was diluted with H₂O and brine (1:1), and washed with ethyl acetate (3×). The combined organics were washed with brine, dried over anhydrous magnesium sulfate (MgSO₄), filtered, and concentrated under reduced pressure. Purification by flash column chromatography (silica gel, gradient: 20% ethyl acetate/hexanes to 50% ethyl acetate/hexanes) yielded 142 mg, 80%) of (3R)-3-benzyloxymethyl-4-{4-[3-(2-methoxybenzyloxy)-propoxy]-phenyl}-5-oxo-piperazine-1-carboxylic acid tert-butyl ester as a clear glassy oil. MS: m/z 591.1 (M+1).

(ii) (3R)-3-Benzyloxymethyl-4-{4-[3-(2-methoxybenzyloxy)-propoxy]-phenyl}-5-oxo-piperazine-1-carboxylic acid tert-butyl ester (288 mg, 0.488 mmole) was dissolved in 15 mL of anhydrous methanol and cooled in an ice bath. Acetyl chloride (0.35 mL, 4.88 mmole) was added neat via syringe in a dropwise fashion over 2-3 minutes. The reaction mixture was stirred for 18 h, while being allowed to gradually warm to room temperature, and then quenched with aqueous saturated sodium bicarbonate (NaHCO₃). The aqueous layer was washed with ethyl acetate (3×). The combined organic layers were washed with brine, dried over anhydrous MgSO₄, filtered, and concentrated under reduced pressure. The crude residue was purified by flash column chromatography (silica gel, gradient: 100% dichloromethane, then 95:5 dichloromethane:methanol to 90:10 dichloromethane:methanol) to afford 195 mg (81%) of (6R)-6-benzyloxymethyl-1-{4-[3-(2-methoxybenzyloxy)-propoxy]-phenyl}-piperazin-2-one as a clear solid gum. MS: m/z 491.2 (M+1).

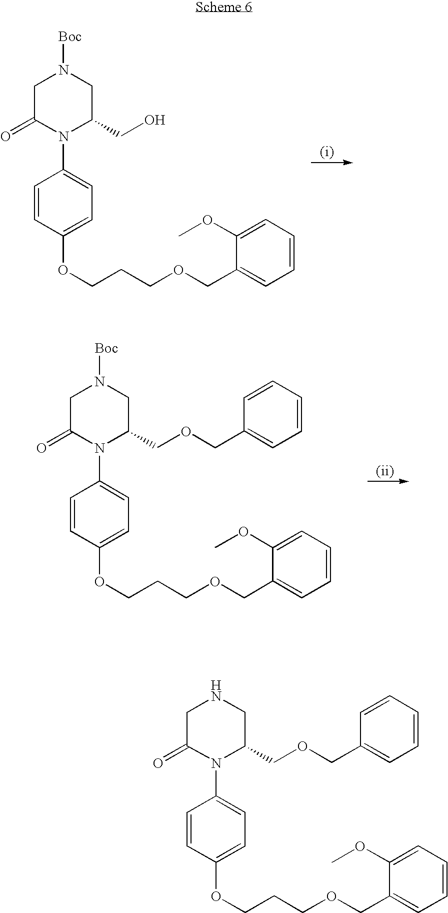

Method D: Formation of Esters as Exemplified by Naphthalene-2-carboxylic acid (2R)-1-(4-[3-(2-methoxy-benzyloxy)-propoxyl]-phenyl)-6-oxopiperazin-2-ylmethyl ester (Scheme 7)

(i) (3R)-3-Hydroxymethyl-4-(4-[3-(2-methoxybenzyloxy)-propoxy]-phenyl)-5-oxo-piperazine-1-carboxylic acid tert-butyl ester (0.200 g, 0.399 mmol) was dissolved in dichloromethane (5 mL) at room temperature. 2-Napthoyl chloride (83.8 mg, 0.44 mmol) followed by triethylamine (60.6 mg, 0.60 mmol) were added and the mixture stirred at room temperature for 18 h. The reaction mixture was diluted with ethyl acetate (20 mL) and layers separated. The organic layer was washed with water (1×20 mL), dried over anhydrous magnesium sulfate, filtered and concentrated under reduced pressure. The crude compound was purified by flash column chromatography (silica gel, gradient: 40 ethyl acetate/hexanes to 75 ethyl acetate/hexanes) to afford 0.201 g (76.8%) of (3R)-4-(4-[3-(2-methoxybenzyloxy)-propoxy]-phenyl)-3-(naphthalene-2-carbonyloxymethyl)-5-oxopiperazine-1-carboxylic acid tert-butyl ester. MS: m/z 655.2 (M+1).

(ii) (3R)-4-(4-[3-(2-Methoxybenzyloxy)-propoxy]-phenyl)-3-(naphthalene-2-carbonyloxymethyl)-5-oxopiperazine-1-carboxylic acid tert-butyl ester (0.16 g, 0.24 mmol) was dissolved in methanol (2 mL) under nitrogen and cooled to 0° C. Acetyl chloride (0.191 g, 2.44 mmol) was added dropwise. The nitrogen inlet needle was removed and the reaction mixture allowed to warm to room temperature over a period of 18 h. The reaction mixture was quenched with saturated sodium bicarbonate. The mixture was extracted with ethyl acetate (3×10 mL). The organic layer was dried over anhydrous magnesium sulfate, filtered and concentrated under reduced pressure. The resultant solid was purified by column chromatography (silica gel, gradient: 100% dichloromethane then 95/5 dichloromethane/methanol to 90/10 dichloromethane/methanol) to afford 17 mg (12.5%) of naphthalene-2-carboxylic acid (2R)-1-(4-[3-(2-methoxy-benzyloxy)-propoxyl]-phenyl)-6-oxopiperazin-2-ylmethyl ester. MS: m/z 555.2 (M+1).

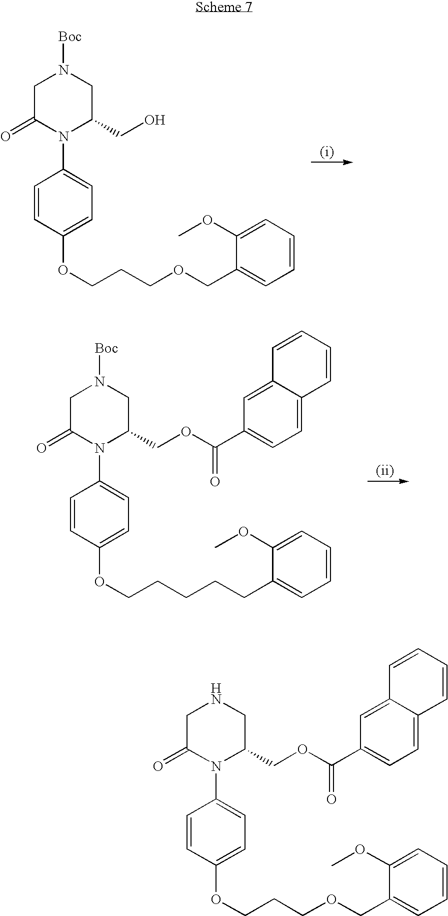

Method E: Formation of Alkylating Agents as Exemplified by 1-(3-Iodopropoxymethyl)-2-methoxybenzene (Scheme 8)

(i) 2-Methoxybenzaldehyde (30 g, 0.22 mol), propane-1,3-diol (18.44 g, 0.24 mol) and benzene (300 mL) were added to a round bottom flask equipped with a Dean-Stark trap. The reaction mixture was heated to reflux for 5 h and then cooled to room temperature. The mixture was diluted with ethyl acetate (300 mL) and layers separated. The organic layer was washed with water (1×300 mL), 1N HCl (1×100 mL), saturated sodium bicarbonate (1×100 mL) and brine (2×100 mL). The organic layer was dried over magnesium sulfate, filtered and concentrated under reduced pressure to obtain 41 g of a yellow solid. The solid was recrystallized from hexanes to obtain 38.31 g (89%) of 2-(2-methoxyphenyl)-[1,3]-dioxane (compound 2). MS: m/z 195.1 (M+1).

(ii) 2-(2-Methoxyphenyl)-[1,3]-dioxane (38.3 g, 0.197 mol) was dissolved in toluene (300 mL) under nitrogen. The mixture was cooled to 0° C. and diisobutylaluminum hydride (61.70 g, 0.433 mol) added slowly. Once the addition was complete, the reaction mixture was allowed to stir for 18 h, slowly warming to room temperature. Ethyl acetate (150 mL) was added to quench excess diisobutylaluminum hydride. A solution of 10% potassium sodium tartrate (800 mL) was added and the mixture stirred for 3 h. Once all salts were dissolved, the layers were separated. The aqueous layer was washed with ethyl acetate (2×400 mL). To the aqueous layer was added 10% sodium hydroxide solution (150 mL) to further break up aluminum salts. The aqueous layer was further extracted with ethyl acetate (2×150 mL). The organic layers were all combined, washed with brine (2×150 mL), dried over anhydrous magnesium sulfate, filtered and concentrated under reduced pressure to afford 37.92 g (98%) of 3-(2-methoxybenzyloxy)-propan-1-ol (compound 3) as a yellow oil. ¹H NMR (400 MHz, CDCl₃) δ 7.28 (dd, J=7.4, 1.1 Hz, 1H), 7.23 (d, J=9.0 Hz, 1H), 6.91 (t, J=7.4 Hz, 1H), 6.84 (d, J=8.3 Hz, 1H), 4.52 (s, 2H), 3.80 (s, 3H), 3.75 (q, J=5.5 Hz, 2H), 3.68 (t, J=5.6 Hz, 2H), 2.61 (t, J=5.6 Hz, 1H), 1.83 (quintet, J=5.6 Hz, 2H).

(iii) 3-(2-Methoxybenzyloxy)-propan-1-ol (37.9 g, 0.193 mol) was dissolved in dichloromethane (300 mL). Pyridine (16.80 g, 0.212 mol), 4-dimethylamino-pyridine (2.35 g, 0.019 mol), and 4-toluenesulfonyl chloride (40.50 g, 0.212 mol) were added at room temperature. The reaction mixture was heated to reflux for 24 h. The mixture was cooled to room temperature and diluted with dichloromethane (400 mL). The layers were separated and the organic layer washed with water (2×200 μL), 1N HCl (2×200 mL), dried over anhydrous magnesium sulfate, filtered and concentrated under reduced pressure to obtain 50 g of solid. The compound was purified by flash column chromatography (15-25% ethyl acetate/hexane mixture) to yield 18.28 g (27%) of toluene-4-sulfonic acid 3-(2-methoxybenzyloxy)-propyl ester as a colorless oil. ¹H NMR (400 MHz, CDCl₃) δ 7.78 (d, J=8.3 Hz, 2H), 7.30 (d, J=8.5 Hz, 2H), 7.26 (dd, J=7.6, 2.0 Hz, 1H), 7.23 (d, J=6.8 Hz, 1H), 6.91 (ddd, J=7.4, 7.4, 1.0 Hz, 1H), 6.85 (d, J=8.3 Hz, 1H), 4.43 (s, 2H), 4.17 (t, J=6.2 Hz, 2H), 3.81 (s, 3H), 3.52 (t, J=6.0 Hz, 2H), 2.40 (s, 3H), 1.94 (quintet, J=6.1 Hz, 2H).

(iv) Toluene-4-sulfonic acid 3-(2-methoxybenzyloxy)-propyl ester (18.22 g, 0.051 mol) was dissolved in acetone (100 mL) under nitrogen. Lithium iodide (10.44 g, 0.077 mol) was added and the mixture heated to reflux for 1 h, cooled to room temperature and filtered through a pad of celite. The celite was washed with acetone and combined with the mother liquor. The organic layer was concentrated under reduced pressure and re-dissolved in dichloromethane. The organic layer was washed with water (2×100 mL), 10% NaS₂O₃ (2×100 mL), brine (2×100 mL), dried over anhydrous magnesium sulfate, filtered and concentrated under reduced pressure to yield 17.03 g (100%) of 1-(3-iodopropoxymethyl)-2-methoxybenzene as a yellow oil that is contaminated with small amount of acetone present. The compound was used without further purification. ¹H NMR (400 MHz, CDCl₃) δ 7.31 (ddd, J=7.3, 1.0, 0.9 Hz, 1H), 7.23 (ddd, J=8.2, 8.2, 1.6 Hz, 1H), 6.91 (ddd, J=7.4, 7.4, 1.0 Hz, 1H), 6.83 (d, J=8.3 Hz, 1H), 4.52 (s, 2H), 3.80 (s, 3H), 3.54 (t, J=5.7 Hz, 2H), 3.28 (t, J=6.8 Hz, 2H), 2.07 (quintet, J=5.9 Hz, 2H).

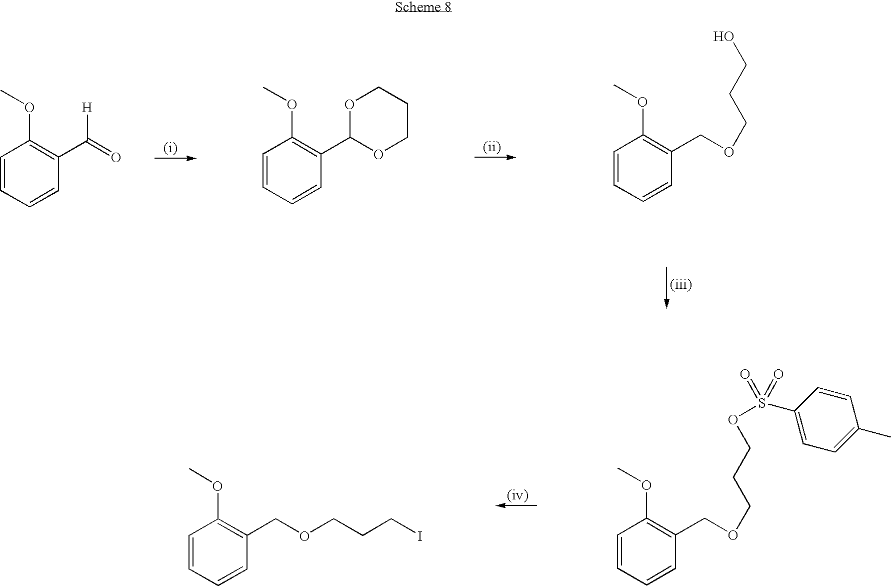

Method F: Preparation of (3R)-3-Hydroxymethyl-5-oxopiperazine-1-carboxylic acid tert-Butyl Ester (Scheme 9)

(i) A 3-necked, 1000 mL round-bottom flask fitted with a N₂ inlet adapter, magnetic stir bar, drying tube, temperature guage, and a septa was charged with methyl (S)-(−)-3-(tert-butoxycarbonyl)-2,2-dimethyl-4-oxazolidine-carboxylate (15.42 g, 59.46 mmole) and 120 mL of anhydrous toluene. The solution was cooled to −78° C. in a dry ice/acetone bath. A solution of diisobutylaluminum hydride in toluene (69.5 mL, 104.1 mmole) was cooled to −78° C. in a separate dry ice/acetone bath and added to the ester solution under N₂ pressure via a steel cannula over a period of 30 min. The rate of addition was adjusted to prevent the reaction mixture from warming above −70° C. After addition was complete, the mixture was stirred at −78° C. for an additional 30 minutes. Excess hydride was quenched by the dropwise addition of 20 mL of pre-chilled (−78° C.) methanol, again keeping the reaction temperature below −70° C. The resulting white slurry was poured into 500 mL of ice-cold 1 N HCl. The aqueous layer was extracted with ethyl acetate (3×300 mL). The combined organic layers were washed with 300 mL 1 N HCl, and brine, dried over anhydrous magnesium sulfate, filtered, and concentrated under reduced pressure to yield (S)-4-formyl-2,2-dimethyl-3-oxazolidinecarboxylic acid tert-butyl ester (14.65 g) as a yellow oil. The residue was dissolved in 200 mL of anhydrous methanol, and the flask was flushed with N₂. N-Benzylglycine ethyl ester (23.0 g, 118.9 mmole) and acetic acid (6.8 mL, 118.9 mmole) were added, and the reaction mixture was cooled in an ice bath. A solution of sodium cyanoborohydride in tetrahydrofuran (100 mL, 100 mmole) was added via a cannula under positive N₂ pressure. The reaction mixture was stirred at room temperature for 18 h. A large excess of solid K₂CO₃ was added until gas evolution ceased. The slurry was concentrated almost to dryness under reduced pressure and the residue was dissolved in 300 mL of dichloromethane. The organic layer was washed with 300 mL of 1:1:1 water/saturated NaHCO₃/brine. The aqueous layer was extracted with ethyl acetate (2×200 mL). The combined organic layers were dried over anhydrous magnesium sulfate, filtered, and concentrated under reduced pressure. Purification of the residue by flash column chromatography (silica gel, gradient: 15% ethyl acetate/hexane to 30% ethyl acetate/hexane) gave 16.83 g (70%) of (S)-4-[(benzylethoxycarbonylmethylamino)-methyl]-2,2-dimethyl-3-oxazolidinecarboxylic acid tert-butyl ester as a clear viscous oil. MS: m/z 407.3 (M+1).

(ii) (S)-4-[(Benzylethoxycarbonylmethylamino)-methyl]-2,2-dimethyl-3-oxazolidinecarboxylic acid tert-butyl ester (16.83 g, 41.41 mmole) was dissolved in 200 mL methanol. An aqueous 6 N HCl solution (20 mL) was added, and the reaction mixture was heated to reflux for 18 h. After cooling to room temperature, the solution was made basic by the addition of saturated aqueous NaHCO₃ and concentrated to one quarter volume under reduced pressure. The resulting slurry was diluted with 300 m]L of 1:1H₂O/brine and extracted with dichloromethane (2×300 mL). The combined organic layers were dried over anhydrous magnesium sulfate, filtered, and concentrated under reduced pressure. Purification of the crude product by flash column chromatography (silica gel, 93/7 dichloromethane/methanol then 90/10 dichloromethane/methanol) gave 6.27 g (69%) of (6R)-4-benzyl-6-hydroxymethyl-piperazin-2-one as a pale yellow solid. [α]_{D}=−34.0 (c=11.4, CDCl₃); MS: m/z 221.0 (M+1).

(iii) 4-Benzyl-6-hydroxymethylpiperazin-2-one (5.57 g, 25.3 mmole) was dissolved in 50 mL of absolute ethanol. Di-tert-butyl carbonate (6.7 g, 30.7 mmole) and 0.50 g of 20% palladium/carbon were added, and the resulting black slurry was shaken under a 50 psi hydrogen atmosphere for 6 hr, periodically maintaining the 50 psi hydrogen pressure. The slurry was filtered through a celite pad, which was rinsed with additional ethanol. The combined filtrates were concentrated under reduced pressure. The crude product was purified by flash column chromatography (silica gel, 100% dichloromethane, then step gradient to 90/10 dichloromethane/methanol) to afford 5.76 g (99%) of (3R)-3-hydroxymethyl-5-oxopiperazine-1-carboxylic acid tert-butyl ester as a white semi-solid. MS: m/z 231.1 (M+1).

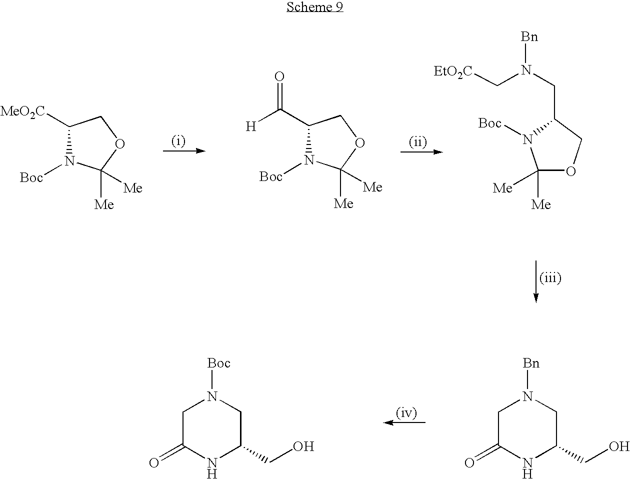

Method G: Preparation of (2R)-1-{4-[3-(2-Methoxybenzyloxy)-propoxy]-phenyl}-2-(naphthalen-2-yloxymethyl)-piperazine (Scheme 10)

(i) A 100 mL round bottom flask was charged with 4-bromophenol (1.87 g, 10.78 mmole), 1-(3-iodopropoxymethyl)-2-methoxybenzene (3.00 g, 9.80 mmole), acetonitrile (20 mL), and potassium carbonate (1.63 g, 11.8 mmole). The resulting slurry was heated to reflux for 18 h. After cooling to room temperature, the mixture was filtered through a celite pad, which was rinsed with additional acetonitrile. The combined filtrates were concentrated under reduce pressure. The residue was dissolved in ethyl acetate and washed with aqueous 10% sodium hydroxide and brine. The organic layer was dried over anhydrous magnesium sulfate, filtered, and concentrated under reduced pressure. Purification by flash column chromatography (silica gel, gradient: 5% ethyl acetate/hexanes to 10% ethyl acetate/hexanes) afforded 1-bromo-4-(3-(2-methoxybenzyloxy)-propyloxy)benzene (2.45 g, 71%) as a clear oil. ¹H NMR (400 MHz, CDCl₃) δ 7.37 (d, J=9.0 Hz, 2H), 7.29-7.25 (m, 2H), 6.94 (t, J=7.4 Hz, 1H), 6.86 (d, J=8.3 Hz, 1H), 6.78 (d, J=9.0 Hz, 2H), 4.57 (s, 2H), 4.07 (t, J=6.2 Hz, 2H), 3.81 (s, 3H), 3.70 (t, J=6.1 Hz, 2H), 2.10 (quintet, J=6.1 Hz, 2H).

(ii) A 50 mL round bottom flask was charged with 1-bromo-4-(3-(2-methoxybenzyloxy)-propyloxy)benzene (1.72 g, 4.88 mmole) and (3R)-3-(naphthalen-2-yloxymethyl)-piperazine-1-carboxylic acid tert-butyl ester (836 mg, 2.44 mmole). The mixture was dissolved in 10 mL of anhydrous toluene and concentrated under reduced pressure to remove residual water. After the flask was equipped with a magnetic stir bar and a reflux condenser, the flask was purged and back-filled with N₂ twice. Palladium bis(tri-tert-butylphosphine) (125 mg, 0.24 mmole), potassium tert-butoxide (301 mg, 2.69 mmole) were added and the flask was again purged and back-filled with N₂. Anhydrous tetrahydrofuran (10 mL) was added, and the resulting orange slurry was heated in a 75° C. oil bath for 18 h. After cooling to room temperature, the mixture was diluted with diethyl ether and filtered through a celite pad, rinsing with diethyl ether. The combined filtrates were concentrated under reduced pressure and purified by flash column chromatography (silica gel, gradient: 10% ethyl acetate/hexanes gradient to 20% ethyl acetate hexanes, then 70/28/2 ethyl acetate/hexanes/methanol) to give (3R)-4-{4-[3-(2-methoxybenzyloxy)-propoxy]-phenyl}-3-(naphthalen-2-yloxymethyl)-piperazine-1-carboxylic acid tert-butyl ester (530 mg, 35%) as a yellow oil. MS: m/z 613.4 (M+1).

(iii) (3R)-4-{4-[3-(2-Methoxybenzyloxy)-propoxy]-phenyl}-3-(naphthalen-2-yloxymethyl)-piperazine-1-carboxylic acid tert-butyl ester (530 mg, 0.864 mmole) was suspended in anhydrous methanol (15 mL) under a N₂ atmosphere, and cooled in an ice bath. Acetyl chloride (0.62 mL, 8.64 mmole) was added dropwise over a 2 min interval. The N₂ inlet needle was removed and the resulting green solution was stirred for 12 h, allowing the ice bath to warm to room temperature. Excess acid was quenched by the addition of an aqueous saturated sodium bicarbonate solution. The aqueous layer was extracted with ethyl acetate (3×50 mL). The combined organic layers were dried over anhydrous magnesium sulfate, filtered, and concentrated under reduced pressure. Purification by flash column chromatography (silica gel, 95/5 dichloromethane/methanol then 90/50 dichloromethane/methanol) provided (2R)-1-{4-[3-(2-methoxybenzyloxy)-propoxy]-phenyl}-2-(naphthalen-2-yloxymethyl)-piperazine (393 mg, 89%) as a brown viscous oil. [α]_{D}=+89.4 (c=1.7, CH₂Cl₂); MS: m/z 513.3 (M+1).

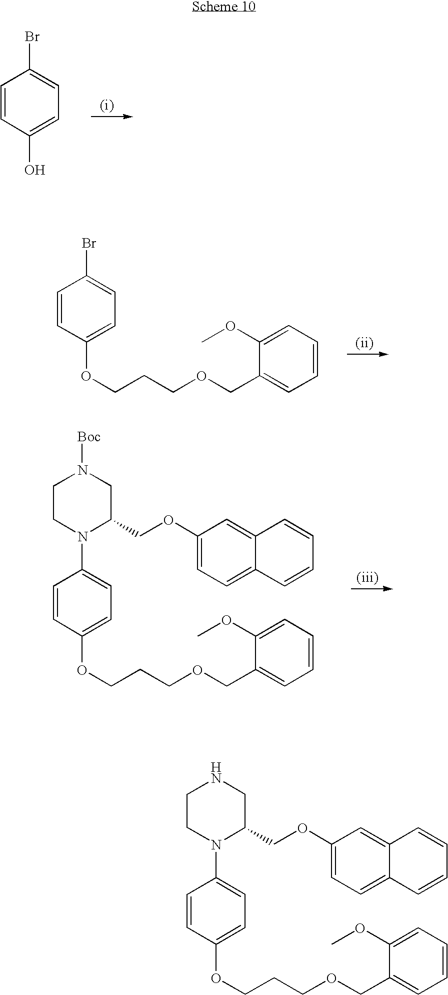

Method H: Preparation of (3R)-4-(4-hydroxyphenyl)-3-(4-methoxybenzyloxymethyl)-piperazine-1-carboxylic acid tert-Butyl Ester (Scheme 11)

(i) N-(tert-Butoxycarbonyl)-L-serine (1.00 g, 4.87 mmole) was dissolved in 10 mL anhydrous dimethylformamide under a N2 atmosphere. The reaction mixture was cooled in an ice bath. Sodium hydride (0.257 g, 10.71 mmole, 95%) was added in portions. The resulting slurry was stirred at 0° C. until gas evolution ceased. 4-Methoxybenzyl chloride (0.73 mL, 5.36 mmole) was added dropwise neat via syringe. The ice bath was removed, and the white suspension was stirred at room temperature for 18 h. The mixture was diluted with water and washed with ethyl acetate (2×). The aqueous layer was acidified to pH 3.5 by the addition of 3 N hydrochloric acid, and extracted with ethyl acetate (3×). The combined organic layers were concentrated under reduced pressure. The residue was redissolved in 50 mL 1:1 tert-butyl methyl ether/hexanes, washed with H₂O (2×) and brine, and dried over anhydrous MgSO₄ to yield N-(tert-butoxycarbonyl)-O-(4-methoxybenzyl)-L-serine (987 mg, 62%) as a yellow viscous oil. MS: m/z 324.2 (M−1, AP−).

(ii) N-(tert-Butoxycarbonyl)-O-(4-methoxybenzyl)-L-serine (987 mg, 3.03 mmole) was dissolved in 10 mL of anhydrous tetrahydrofuran. HBTU (1.27 g, 3.34 mmole), i-Pr₂NEt (0.80 mL, 4.55 mmole), and N-benzylglycine ethyl ester (0.68 mL, 3.64 mmole) were added, and the resulting white suspension was stirred at room temperature for 18 hr. The reaction mixture was diluted with 50 mL of dichloromethane and washed with aqueous 10% citric acid and saturated NaHCO₃. The organic layer was dried over MgSO₄ and concentrated under reduced pressure. Purification by flash column chromatography (silica gel, gradient: 20% ethyl acetate/hexanes to 30% ethyl acetate/hexanes) afforded {benzyl-[(S)-2-tert-butoxycarbonylamino-3-(4-methoxybenzyloxy)-propionyl]-amino}-acetic acid ethyl ester (1.40 g, 92%) as a clear viscous oil. MS: m/z 501.3 (M+H).

(iii) {Benzyl-[(S)-2-tert-butoxycarbonylamino-3-(4-methoxybenzyloxy)-propionyl]-amino}-acetic acid ethyl ester (1.40 g, 2.79 mmole) was dissolved in 20 mL of methanol and cooled in an ice bath. Acetyl chloride (1.0 mL, 13.94 mmole) was added dropwise. The N₂ inlet needle was removed and the reaction mixture was allowed to gradually warn to room temperature over 18 h. Excess acid was quenched by the addition of aqueous saturated NaHCO₃, and the solvent volume was reduced by half under reduced pressure. The residue was portioned between water and ethyl acetate (3×). The combined organics were dried over MgSO₄, filtered, and concentrated under reduced pressure. Purification by flash column chromatography (silica gel, gradient: 85/13/2 ethyl acetate/hexanes/methanol to 98/2 ethyl acetate/methanol) provided 551 mg (56%) of (3S)-1-benzyl-3-(4-methoxybenzyloxymethyl)-piperazine-2,5-dione as a white solid. MS: m/z 355.2 (M+H).

(iv) (3S)-1-Benzyl-3-(4-methoxybenzyloxymethyl)-piperazine-2,5-dione (550 mg, 1.55 mmole) was dissolved in 6 mL of anhydrous tetrahydrofuran. Lithium aluminum hydride (147 mg, 3.88 mmole) was added in portions. The resulting black suspension was refluxed for 3 hr. After cooling to room temperature, 10 mL of ethyl acetate was carefully added, followed by 20 mL of aqueous 10% potassium sodium tartrate. The biphasic mixture was stirred vigorously for 30 min to dissolve aluminum salts. The aqueous layer was extracted with ethyl acetate (3×25 mL). The combined organics were dried over MgSO₄, filtered, and concentrated under reduced pressure. Purification by flash column chromatography (silica gel, gradient: 95/5/0.2 ethyl acetate/methanol/NH₄OH to 90/10/0.2 ethyl acetate/methanol/NH₄OH) afforded 498 mg (98%) of (3R)-1-benzyl-3-(4-methoxybenzyloxymethyl)-piperazine as a yellow semi-solid. MS: m/z 327.2 (M+H).

(v) An oven-dried reaction tube was cooled under vacuum, back-filled with N₂, and charged with (3R)-1-benzyl-3-(4-methoxybenzyloxymethyl)-piperazine (300 mg, 0.92 mmole), 4-benzyloxy-1-bromobenzene (290 mg, 1.10 mmole), sodium tert-butoxide (120 mg, 1.30 mmole), and palladium bis(tri-tert-butylphosphine) (24 mg, 0.05 mmole). The tube was purged and back-filled with N₂. Anhydrous toluene (2 mL) was added and the dark orange solution was heated in a 60° C. oil bath for 18 h. After cooling to room temperature, the mixture was diluted with water and extracted with ethyl acetate (2×). The combined organics were dried over MgSO₄, filtered, and concentrated under reduced pressure. The residue was purified by flash column chromatography (silica gel, gradient: 10% ethyl acetate/hexanes to 20% ethyl acetate/hexanes) to yield 449 mg (96%) of (2R)-4-benzyl-1-(4-benzyloxyphenyl)-2-(4-methoxybenzyloxymethyl)-piperazine as a light orange solid. MS: m/z 509.3 (M+H).

(vi)(2R)-4-Benzyl-1-(4-benzyloxyphenyl)-2-(4-methoxybenzyloxymethyl)-piperazine (9.30 mmole) was dissolved in 100 mL of tetrahydrofuran and 10 mL of absolute ethanol, and treated with di-tert-butyl dicarbonate (5.98 g, 27.4 mmole) and 20% Pd(OH)₂/C (1.0 g). The resulting black suspension was shaken under a 50 psi hydrogen atmosphere for 18 h. The mixture was filtered through a celite pad and concentrated under reduced pressure. Purification by flash column chromatography (silica gel, gradient: 20% ethyl acetate/hexanes to 40% ethyl acetate/hexanes) gave 7.31 g (93%) of (3R)-4-(4-hydroxyphenyl)-3-(4-methoxybenzyloxymethyl)-piperazine-1-carboxylic acid tert-butyl ester as a white solid. MS: m/z 429.2 (M+H).

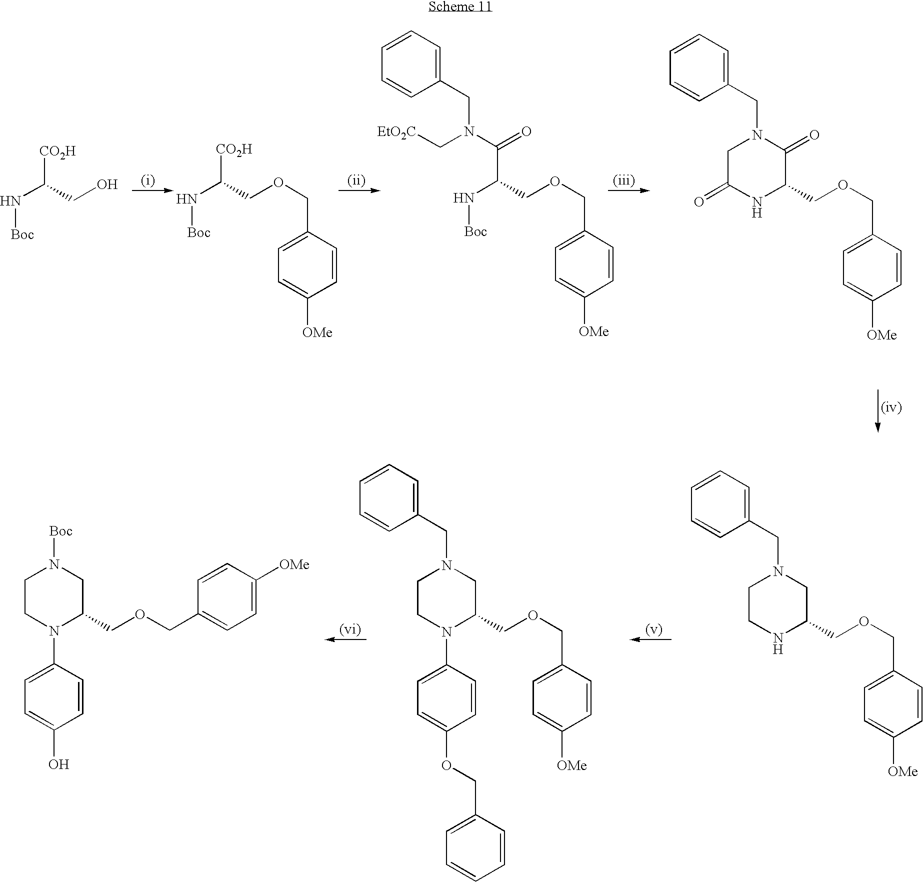

Method I: Preparation of (3R)-3-(Naphthalen-2-yloxymethyl)-piperazine-1-carboxylic acid tert-Butyl Ester (Scheme 12)

(i) A oven-dried, 25 mL round bottom flask was cooled under a N₂ stream and charged with (3R)-3-hydroxymethyl-5-oxopiperazine-1-carboxylic acid tert-butyl ester (100 mg, 0.434 mmole), 2-naphthol (94 mg, 0.65 mmole), and 5 mL anhydrous dichloromethane. Polymer-supported triphenylphosphine (383 mg, 0.521 mmole, 1.36 mmole/g loading) was added, and the resulting slurry was stirred at room temperature for 20 min. The mixture was cooled in an ice bath, and diisopropyl azodicarboxylate (141 mg, 0.695 mmole) was added dropwise via a syringe. The yellow slurry was stirred overnight, allowing to warm to room temperature. The resin was collected on a medium frit, rinsing with dichloromethane (3×4 mL). The combined filtrates were washed with aqueous 1 N HCl and 10% sodium hydroxide, dried over anhydrous magnesium sulfate, filtered, and concentrated under reduced pressure. Purification by flash column chromatography (silica gel, gradient: 60% ethyl acetate/hexanes to 100% ethyl acetate) afforded (3R)-3-(naphthalen-2-yloxymethyl)-5-oxopiperazine-1-carboxylic acid tert-butyl ester (76.3 mg, 49%) as a clear viscous oil. MS: m/z 257.0 (M-C₅H₉O₂, AP+), 355.2 (M−H, AP−).

(ii) (3R)-3-(Naphthalen-2-yloxymethyl)-5-oxopiperazine-1-carboxylic acid tert-butyl ester (1.55 g, 4.34 mmole) was dissolved in 40 mL anhydrous THF under a N₂ atmosphere. A 2.0 M solution of borane dimethyl sulfide complex in THF (6.5 mL, 13.0 mmole) was added via syringe, and the reaction mixture was heated in a 50° C. oil bath for 2 h. After cooling to room temperature, excess hydride was quenched by the careful addition of methanol. The mixture was concentrated under reduced pressure, and then redissolved in 75 mL of methanol. An aqueous solution of 10% potassium sodium tartrate (50 mL) was added, and the resulting slurry was heated at reflux for 2 h and then stirred at room temperature for 18 h. The mixture was concentrated under reduced pressure and partitioned between water and ethyl acetate (3×). The combined organic layers were washed with brine, dried over anhydrous magnesium sulfate, filtered, and concentrated under reduced pressure. Purification by flash column chromatography (silica gel, 65/33/2 ethyl acetate/hexanes/methanol then 70/28/2 acetate/hexanes/methanol) provided (3R)-3-(naphthalen-2-yloxymethyl)-piperazine-1-carboxylic acid tert-butyl ester (1.149 g, 77%) as a clear glassy solid. MS: m/z 343.2 (M+1).

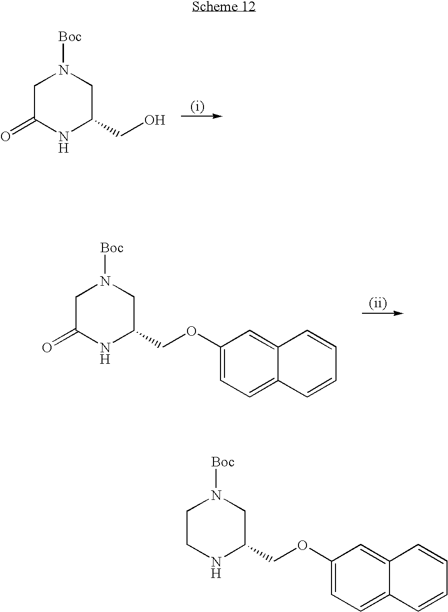

Method J: Alkylation of 4-Hydroxyphenyl piperazinones (Scheme 13)

(i) (3R)-3-Hydroxymethyl-4-(4-hydroxy-phenyl)-5-oxo-piperazine-1-carboxylic acid tert-butyl ester (3.0 g, 9.3 mmol) was dissolved in acetonitrile (50 mL), and cesium carbonate (6.06 g, 18.61 mmol) and benzyl bromide (2.03 g, 11.63 mmol) were added. The suspension was heated to reflux for 2 h. The mixture was concentrated under reduced pressure and purified by column chromatography (silica gel, 1:1 hexanes/ethyl acetate) to afford (3R)-4-(4-benzyloxyphenyl)-3-hydroxymethyl-5-oxopiperazine-1-carboxylic acid tert-butyl ester (2 g, 52.1%). MS: m/z 413.2 (M+1).

(ii) (3R)-4-(4-Benzyloxyphenyl)-3-hydroxymethyl-5-oxo-piperazine-1-carboxylic acid tert-butyl ester (1.5 g, 3.64 mmol) was dissolved in dichloromethane (3 mL) and triethylamine (1.1 g, 10.9 mmol) added. The mixture was cooled to −78° C. and trifluoromethanesulfonic anhydride (2 g, 7.2 mmol) was added dropwise via syringe. The mixture was kept at −78° C. for 20 minutes and then warmed to 0° C. for 30 minutes. Water (5 mL) and dichloromethane (15 mL) were added, and the mixture was warmed to room temperature. The layers were separated, and the organic layer was dried over anhydrous magnesium sulfate, filtered, and concentrated under reduced pressure to yield (5R)-4-(4-benyloxy-phenyl)-3-oxo-5-trifluoromethanesulfonyloxymethyl-piperazine-1-carboxylic acid tert-butyl ester (1.80 g, 90.9%). The compound was used without further purification. MS: m/z 545.54 (M+1).

(iii) (5R)-4-(4-Benyloxyphenyl)-3-oxo-5-trifluoromethanesulfonyloxymethyl-piperazine-1-carboxylic acid tert-butyl ester (1.80 g, 3.31 mmol) was dissolved in acetonitrile. Cesium carbonate (1.29 g, 3.97 mmol) and 2-naphthol (0.47 g, 3.3 mmol) were added, and the mixture was heated to reflux for 1 h. The mixture was concentrated under reduced pressure and re-dissolved in ethyl acetate (50 mL). The organic layer was washed with water (2×50 mL), dried over anhydrous MgSO₄, filtered and concentrated under reduced pressure. The residue was purified by flash column chromatography (silica gel, gradient: 0% ethyl acetate/hexanes to 100% ethyl acetate) to afford (3R)-4-(4-benzyloxyphenyl)-3-(naphthalene-2-yloxymethyl)-5-oxopiperazine-1-carboxylic acid tert-butyl ester (1.2 g, 67.4%). MS: m/z 539.64 (M+1).

(iv) (3R)-4-(4-Benzyloxyphenyl)-3-(naphthalene-2-yloxymethyl)-5-oxopiperazine-1-carboxylic acid tert-butyl ester (0.65 g, 1.21 mmol) was dissolved in a mixture of THF and methanol (1:1, 16 mL) and treated with 20% palladium on carbon (0.1 g). The resulting black suspension was shaken under a 50 psi atmosphere of hydrogen gas for 6 h. The mixture was filtered through a pad of celite, and then concentrated under reduced pressure. Purification by flash column chromatography (silica gel, 0% ethyl acetate/hexanes to 100% ethyl acetate) afforded (3R)-4-(4-hydroxy-phenyl)-3-(naphthalene-2-ylmethoxy)-5-oxopiperazine-1-carboxylic acid tert-butyl ester (0.36 g, 66.5%). MS: m/z 448.2 (M+1).

(v) (3R)-4-(4-Hydroxyphenyl)-3-(naphthalene-2-yloxymethyl)-5-oxopiperazine-1-carboxylic acid tert-butyl ester (0.72 g, 1.61 mmol) was dissolved in acetonitrile (100 mL). Potassium carbonate (0.66 g, 4.8 mmol) and 3-bromo-1-propanol (0.56 g, 4 mmol) were added. The mixture was heated to reflux for 18 h. The mixture was cooled to room temperature, filtered through a pad of celite, concentrated under reduced pressure, and purified by flash column chromatography (silica gel, gradient: 0% ethyl acetate/hexanes to 100% ethyl acetate) to afford (3R)-4-[4-(3-hydroxypropoxy)-phenyl]-3-(naphthalene-2-yloxymethyl)-5-oxopiperazine-1-carboxylic acid tert-butyl ester (0.45 g, 55.5%) as a white solid. MS: m/z 507.24 (M+1).

(vi) To a suspension of sodium hydride (0.024 g, 0.59 mmol, 60% suspension in mineral oil) in N,N-dimethylformamide (2 mL) was added at room temperature, a solution of (3R)-4-[4-(3-hydroxypropoxy)-phenyl]-3(naphthalene-2-yloxymethyl)-5-oxopiperazine-1-carboxylic acid tert-butyl ester (0.0.075 g, 0.15 mmol) in N,N-dimethylformamide (1 mL). 15-Crown-S (1 drop) was added to the mixture, and the mixture was stirred for 1 h before 2-fluorobenzyl bromide (0.035 g, 0.18 mmol) was added. The mixture was heated to 60° C. for 2 h. The mixture as cooled to room temperature and concentrated under reduced pressure. The resultant residue was purified by flash column chromatography (silica gel, 0% ethyl acetate/hexanes to 100% ethyl acetate) to afford (3R)-4-(4-[3-(2-fluorobenzyloxy)-propoxy]-phenyl)-3-(naphthalene-2-yloxymethyl)-5-oxopiperazine-1-carboxylic acid tert-butyl ester (0.03 g, 33%). MS: m/z 615.28 (M+1).

(vii) (3R)-4-(4-[3-(2-Fluorobenzyloxy)-propoxy]-phenyl)-3-(naphthalene-2-yloxymethyl)-5-oxopiperazine-1-carboxylic acid tert-butyl ester (0.03 g, 0.049 mmol) was dissolved in methanol (2 mL) under nitrogen and cooled to 0° C. Acetyl chloride (0.019 g, 0.24 mmol) was added dropwise. The nitrogen inlet needle was removed, and the reaction mixture was allowed to warm to room temperature over a period of 18 h. The reaction mixture was quenched with saturated sodium bicarbonate. The mixture was extracted with ethyl acetate (3×10 mL). The organic layer was dried over anhydrous magnesium sulfate, filtered and concentrated under reduced pressure. The resultant solid was purified by flash column chromatography (silica gel, gradient: 100% dichloromethane then 95/5 dichloromethane/methanol to 90/10 dichloromethane/methanol) to afford 15 mg (59.7%) of (6R)-1-(4-[3-(2-fluoro-benzyloxy)-propoxy]-phenyl)-6-(naphthalene-2-yloxymethyl)-piperazin-2-one. MS: m/z 515.2 (M+1).

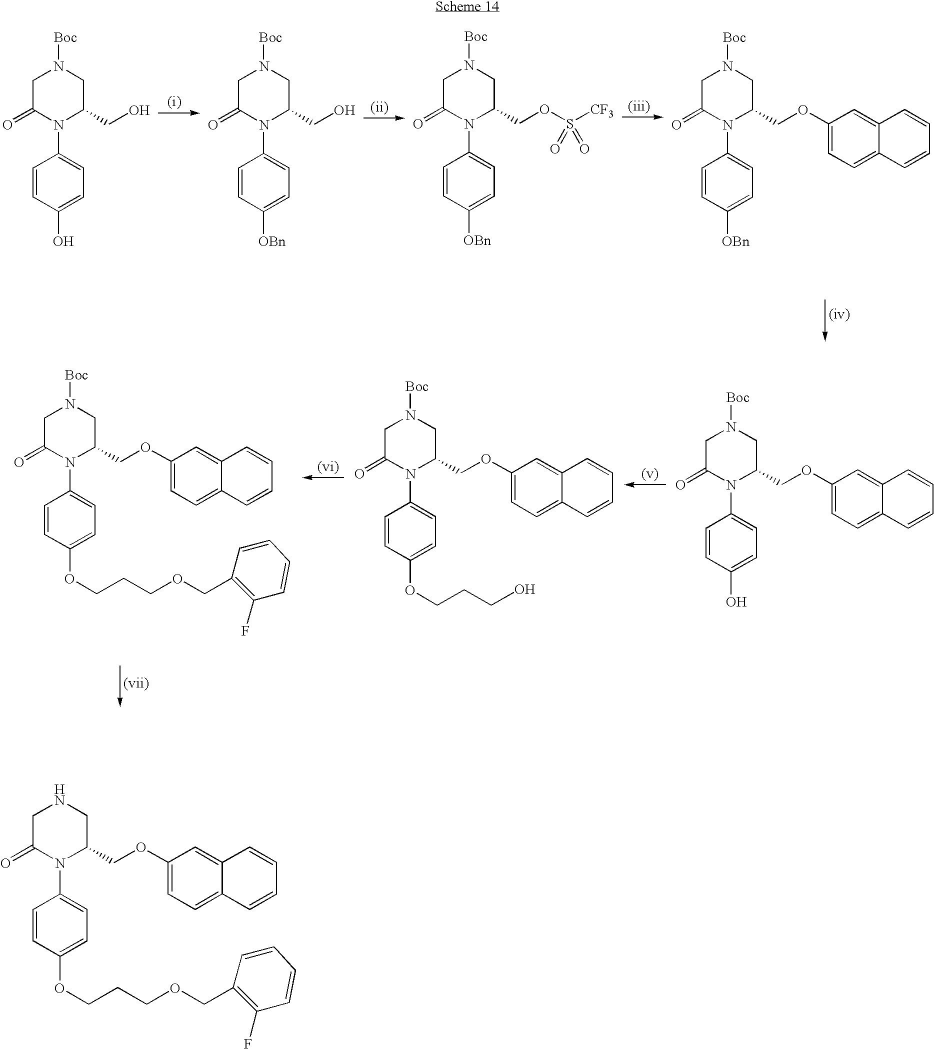

Method K: Preparation of (3S)-3-Aminomethyl-4-(4-[3-(2-methoxy-benzyloxy)-propoxy]-phenyl)-5-oxopiperazine-1-carboxylic acid tert-Butyl Ester (Scheme 15)

(i) (3R)-3-Hydroxymethyl-4-(4-[3-(2-methoxybenzyloxy)-propoxy]-phenyl)-5-oxo-piperazine-1-carboxylic acid tert-butyl ester (2.13 g, 4.26 mmol) was dissolved in dichloromethane (25 mL) and triethylamine (1.29 g, 12.77 mmol) was added. The reaction mixture was cooled to −78° C. in a dry ice/acetone bath under nitrogen and trifluoromethanesulfonic anhydride (2.40 g, 8.51 mmol) added via syringe. The cold bath was removed, and mixture was stirred for 10 minutes while warming to room temperature. Thin layer chromatography analysis (1:1 ethyl acetate/hexanes) indicated reaction was complete within 15 minutes. The mixture was diluted with dichloromethane and layers separated. The organic layer was washed with water (3×25 mL), dried over anhydrous magnesium sulfate, filtered, and concentrated under reduced pressure to afford (5R)-4-(4-[3-(2-methoxybenzyloxy)-propoxy]-phenyl)-3-oxo-5-trifluoromethanesulfonyloxymethyl-piperazine-1-carboxylic acid tert-butyl ester (2.65 g, 98.44%) as an orange oil. Product was used without further purification. MS: m/z 633.1 (M+1).

(ii) (5R)-4-(4-[3-(2-Methoxybenzyloxy)-propoxy]-phenyl)-3-oxo-5-trifluoromethanesulfonyloxymethyl-piperazine-1-carboxylic acid tert-butyl ester (1.9 g, 3 mmol) was dissolved in N,N-dimethylformamide (8 mL), and sodium azide (0.29 g, 4.5 mmol) was added. The reaction mixture was stirred at room temperature for 18 h. The mixture was diluted with ethyl acetate (30 mL) and water added. The layers were separated and the organic layer washed with brine (2×20 mL), dried over anhydrous magnesium sulfate, filtered and concentrated under reduced pressure to yield a golden oil. The oil was purified by flash column chromatography (silica gel, 45% ethyl acetate/hexanes) to afford 0.552 g (35%) of (3R)-3-azidomethyl-4(4-[3-(2-methoxybenzyloxy)-propoxy]-phenyl)-5-oxopiperazine-1-carboxylic acid tert-butyl ester. MS: m/z 526.2 (M+1).

(iii) (3R)-3-Azidomethyl-4(4-[3-(2-methoxybenzyloxy)-propoxy]-phenyl)-5-oxo-piperazine-1-carboxylic acid tert-butyl ester (0.51 g, 0.97 mmol) was dissolved in tetrahydrofuran (50 mL) and Raney nickel added (0.2 g). The mixture was shaken under an atmosphere of hydrogen (4295 psi/mole) for 16 h. The mixture was filtered through a pad of celite and concentrated under reduced pressure to afford 0.471 g (96.9%) of (3S)-3-aminomethyl-4-(4-[3-(2-methoxy-benzyloxy)-propoxy]-phenyl)-5-oxopiperazine-1-carboxylic acid tert-butyl ester. The compound was used without further purification. MS: m/z 500.2 (M+1).

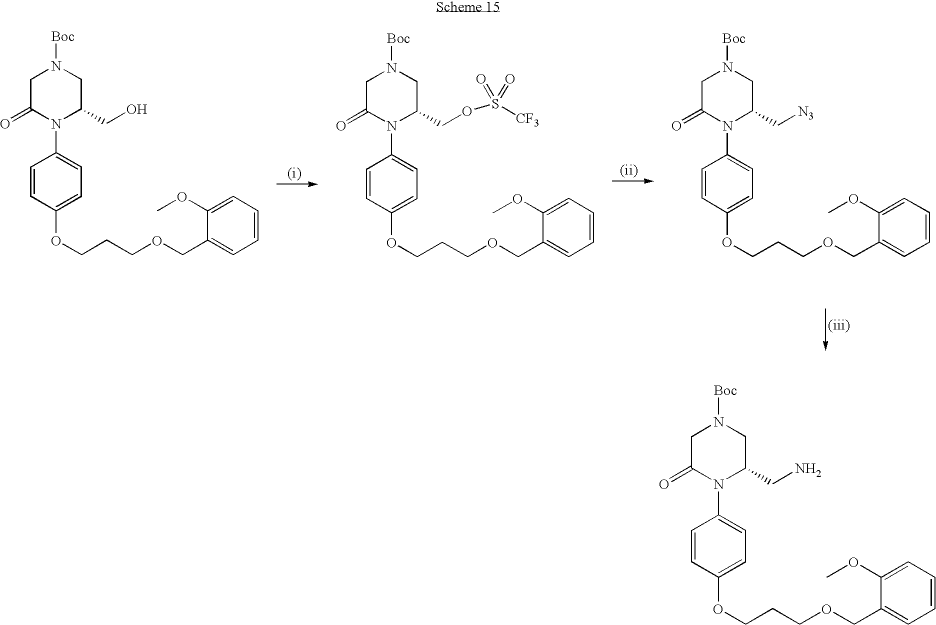

Method L: Preparation of Amides (Scheme 16)

(i) (3S)-3-Aminomethyl-4-(4-[3-(2-methoxybenzyloxy)-propoxy]-phenyl)-5-oxo-piperazine-1-carboxylic acid tert-butyl ester (0.1 g, 0.2 mmol) was dissolved in dichloromethane (5 mL) at room temperature. 2-Methoxybenzoyl chloride (41 mg, 0.24 mmol) followed by triethylamine (40.5 mg, 0.4 mmol) were added, and the mixture stirred at room temperature for 18 h. The reaction mixture was diluted with ethyl acetate (20 mL) and layers separated. The organic layer was washed with water (1×20 mL), dried over anhydrous magnesium sulfate, filtered and concentrated under reduced pressure. The crude compound was purified by flash column chromatography (silica gel, gradient: 40% ethyl acetate/hexanes to 75% ethyl acetate/hexanes) to afford 0.126 g (99%) of (3S)-3-[(2-methoxybenzoylamino)-methyl]-4-(4-[3-(2-methoxybenzyloxy)-propoxy]-phenyl)-5-oxopiperazine-1-carboxylic acid tert-butyl ester. MS: m/z 634.2 (M+1)

(ii) (3S)-3-[(2-Methoxybenzoylamino)-methyl]-4-(4-[3-(2-methoxybenzyloxy)-propoxy]-phenyl)-5-oxopiperazine-1-carboxylic acid tert-butyl ester (0.126 g, 0.2 mmol) was dissolved in methanol (2 mL) under nitrogen and cooled to 0° C. Acetyl chloride (0.191 g, 2.44 mmol) was added dropwise. The nitrogen inlet needle was removed, and the reaction mixture was allowed to warm to room temperature over a period of 18 h. The reaction mixture was quenched with saturated sodium bicarbonate. The mixture was extracted with ethyl acetate (3×10 mL). The organic layer was dried over anhydrous magnesium sulfate, filtered and concentrated under reduced pressure. The resultant solid was purified by flash column chromatography (silica gel, gradient: 100% dichloromethane then 95/5 dichloromethane/methanol to 90/10 dichloromethane/methanol) to afford 41 mg (38%) of 2-methoxy-N-([2R]-1-(4-[3-(2-methoxybenzyloxy)-propoxy]-phenyl)-6-oxopiperazin-2-ylmethyl)-benzamide. MS: m/z 534.2 (M+1).

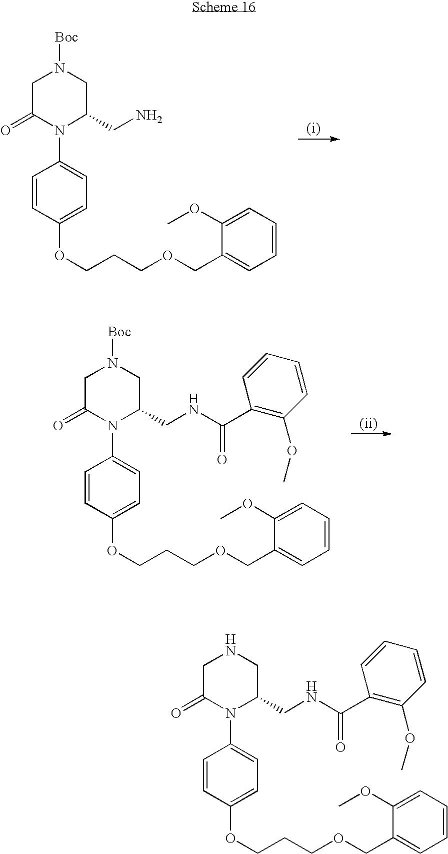

Method M: Preparation of Ureas (Scheme 17)

(i) (3S)-3-Aminomethyl-4-(4-[3-(2-methoxybenzyloxy)-propoxy]-phenyl)-5-oxo-piperazine-1-carboxylic acid tert-butyl ester (0.1 g, 0.2 mmol) was dissolved in dichloromethane (2 mL). 3,4-Dichlorophenylisocyanate (0.045 g 0.24 mmol) was added and the mixture stirred at room temperature for 18 h. Resin quench reagent (trisamine resin (100 mg, (loading: 3.4 mmol/g) was added to the mixture, and the resulting slurry was allowed to stir for an additional 18 h. The mixture was filtered and evaluated by HPLC to confirm acceptable purity (70%). (3S)-3-[3-(3,4-Dichlorophenyl)-ureidomethyl]-4-(3-(2-methoxy-benzyloxy)-propoxy]-phenyl)-5-oxopiperazine-1-carboxylic acid tert-butyl ester (0.137 g, 100%) was used without further purification and used immediately in the next step. MS: m/z 687.2, 689.2 (M+1).

(iii) (3S)-3-[3-(3,4-Dichlorophenyl)-ureidomethyl]-4-(3-(2-methoxybenzyloxy)-propoxy]-phenyl)-5-oxopiperazine-1-carboxylic acid tert-butyl ester (0.137 g, 0.2 mmol)) was dissolved in tetrahydrofuran (2 mL) under nitrogen, and 4M HCl/dioxane (72.9 mg, 2 mmol) was added at room temperature. The reaction mixture was stirred for 18 h. The mixture was diluted with dichloromethane (3 mL), and excess acid was quenched by the addition of solid bicarbonate. The mixture was purified by flash column chromatography (gradient: 100% dichloromethane then 95/5 dichloromethane/methanol to 90/10 dichloromethane/methanol) to afford 60 mg (51%) of 1-(3,4-dichloro-phenyl)-3-([2R]-1-(4-[3-(2-methoxybenzyloxy)-propoxy]-phenyl)-6-oxopiperazin-2-ylmethyl) urea. MS: m/z 587.2, 589.2 (M+1).

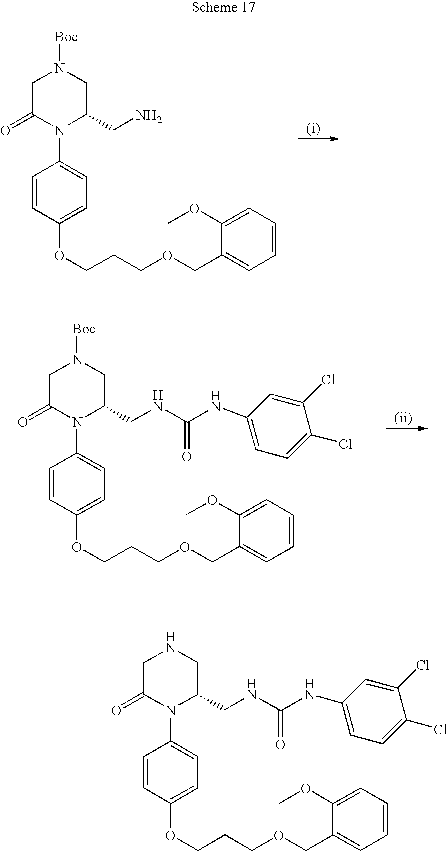

Method N: Preparation of Sulfonamides (Scheme 18)

(i) (3S)-3-Aminomethyl-4-(4-[3-(2-methoxybenzyloxy)-propoxy]-phenyl)-5-oxo-piperazine-1-carboxylic acid tert-butyl ester (0.1 g, 0.2 mmol) was dissolved in dichloromethane (2 mL) and triethylamine (40.51 mg, 0.4 mmol) added at room temperature. 3,4-Dichlorobenzenesulfonyl chloride (59 mg, 0.24 mmol) was added and the reaction mixture was allowed to stir at room temperature for 18 h. Trisamine resin (100 mg, (loading: 3.4 mmol/g)) was added, and the mixture was stirred for 18 h at room temperature. The mixture was filtered and evaluated by HPLC to confirm acceptable purity (70% pure). (3R)-3-[(3,4-Dichlorobenzenesulfonylamino)-methyl]-4-(4-[3-(2-methoxybenzyloxy)-propoxy]-phenyl)-5-oxopiperazine-1-carboxylic acid tert-butyl ester (0.141 g, 100%) was used without further purification. MS: m/z 706.1, 708.1 (M+1).

(ii) (3R)-3-[(3,4-Dichlorobenzenesulfonylamino)-methyl]-4-(4-[3-(2-methoxy-benzyloxy)-propoxy]-phenyl)-5-oxopiperazine-1-carboxylic acid tert-butyl ester (0.141 g, 0.2 mmol) was dissolved in tetrahydrofuran (2 mL). 4M HCl in dioxane (72.99 mg, 2 mmol) was added, and the mixture was allowed to stir at room temperature for 18 h. Dichloromethane (3 mL) and saturated sodium bicarbonate (5 mL) was added to the mixture and the layers separated. The organic layer was purified by flash column chromatography (gradient: 100% dichloromethane, then 95/5 dichloromethane/methanol to 90/10 dichloromethane/methanol) to afford 36 mg (29.55%) of (3,4-dichloro-N-(2R)-1-(4-[3-(2-methoxybenzyloxy)-propoxy)-phenyl)-6-oxopiperazin-2-ylmethyl)-benzenesulfonamide. MS: m/z 606.1, 608.1 (M+1).

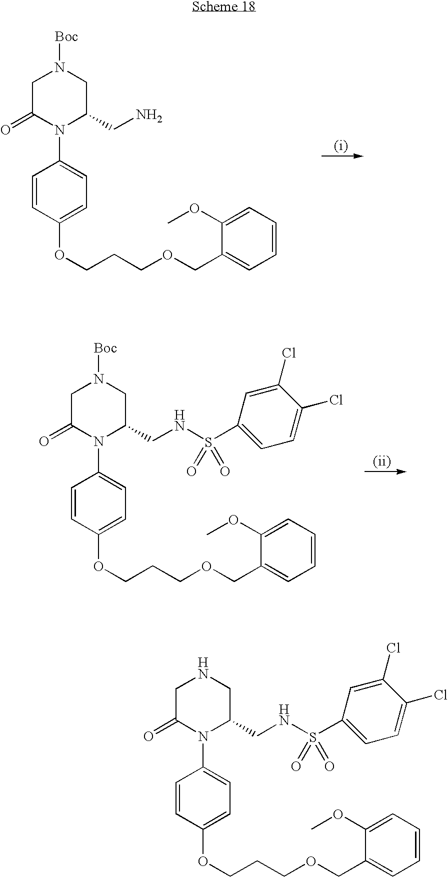

Method O: Preparation of Alkylated Tetrahydroquinoline Analogs (Scheme 19)

(i) (5R)-4-{4-[3-(2-Methoxybenzyloxy)-propoxy]-phenyl}-3-oxo-5-(quinolin-7-yloxymethyl)-piperazine-1-carboxylic acid tert-butyl ester (1.11 g, 1.76 mmole), prepared as described in General Procedure 1, and nickel(II) chloride hexahydrate (250 mg, 1.06 mmole) was dissolved in methanol (20 mL). The light green solution was cooled in an ice bath. Sodium borohydride (266 mg, 7.04 mmole) was added in portions over 5 min to control the exothermic gas evolution. The black suspension was stirred at 0° C. for 2 hr. An additional 87 mg of sodium borohydride was added, and the suspension was stirred for an additional 30 min. The green solution was poured into a 1:1 mixture of aqueous saturated ammonium chloride and water. The aqueous layer was washed with ethyl acetate (3×50 mL). The combined organic layers were washed with water and brine, dried over anhydrous magnesium sulfate, filtered, and concentrated under reduced pressure. Purification by flash column chromatography (silica gel, gradient: 40% ethyl acetate/hexanes to 75% ethyl acetate hexanes) afforded 900 mg (81%) of (5R)-4-{4-[3-(2-methoxybenzyloxy)-propoxy]-phenyl}-3-oxo-5-(1,2,3,4-tetrahydroquinolin-7-yloxymethyl)-piperazine-1-carboxylic acid tert-butyl ester as a off-white solid foam. MS: m/z 632.2 (M+1).

(ii) (5R)-4-{4-[3-(2-Methoxybenzyloxy)-propoxy]-phenyl}-3-oxo-5-(1,2,3,4-tetrahydroquinolin-7-yloxymethyl)-piperazine-1-carboxylic acid tert-butyl ester (360 mg, 0.570 mmole) was dissolved in anhydrous acetonitrile (3 mL). Sodium carbonate (91 mg, 0.86 mmole), potassium iodide (19 mg, 0.11 mmole), and 2-bromoethanol (121 μL, 1.71 mmole) were added, and the suspension was heated at reflux for 18 h. An additional 100 μL of 2-bromoethanol was added, and the reaction was heated at reflux for an additional 24 h. After cooling to room temperature, the mixture was diluted with acetonitrile, filtered through a celite plug, and concentrated under reduced pressure. Purification by flash column chromatography (silica gel, gradient: 30% ethyl acetate/hexanes to 80% ethyl acetate hexanes) yielded 291 mg (76%) of (3R)-3-[1-(2-hydroxyethyl)-1,2,3,4-tetrahydroquinolin-7-yloxymethyl]-4-{4-[3-(2-methoxybenzyloxy)-propoxy]-phenyl}-5-oxopiperazine-1-carboxylic acid tert-butyl ester as a clear glass. MS: m/z 676.3 (M+1).

(iii) (3R)-3-[1-(2-Hydroxyethyl)-1,2,3,4-tetrahydroquinolin-7-yloxymethyl]-4-{4-[3-(2-methoxybenzyloxy)-propoxy]-phenyl}-5-oxopiperazine-1-carboxylic acid tert-butyl ester (96 mg, 0.14 mmole) was dissolved in anhydrous methanol (6 mL). The solution was cooled in an ice bath and acetyl chloride (101 μL, 1.42 mmole) was added dropwise. The N₂ inlet needle was removed, and the reaction mixture was stirred for 18 h, slowly allowing the ice bath to warm to room temperature. Solid potassium carbonate was added and the suspension was vigorously stirred for 1 h. The mixture was diluted with 20 mL of dichloromethane, filtered through a celite plug, and concentrated under reduced pressure. Purification by flash column chromatography (silica gel, gradient: 100:0 dichloromethane/methanol to 90/10 dichloromethane/methanol) afforded 42 mg (51%) of acetic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester as a yellow viscous oil. MS: m/z 618.3 (M+1).

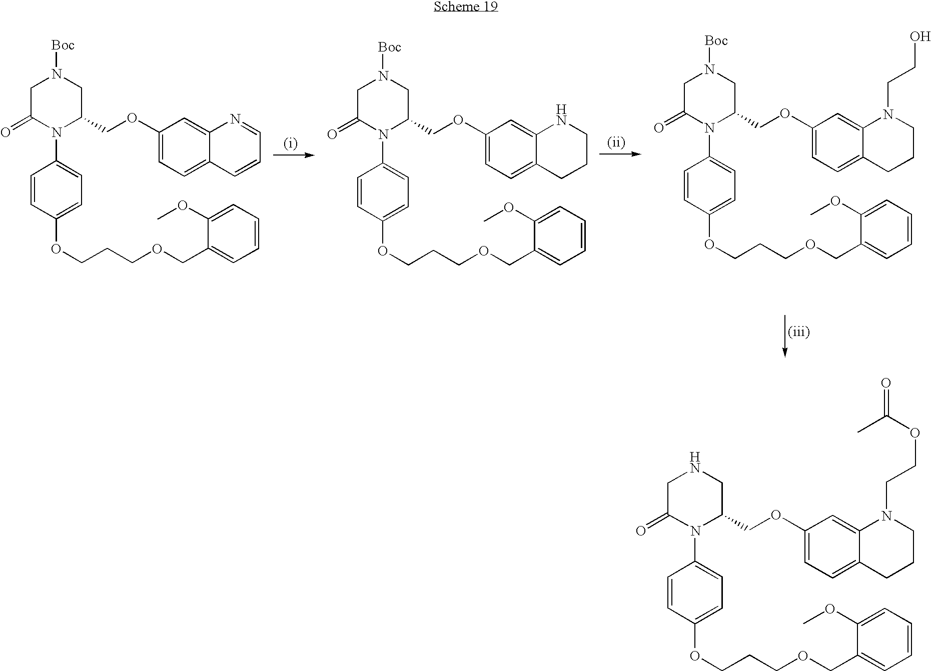

Method P: Preparation of Alkylated Indole Analogs (Scheme 20)

(i) 6-Benzyloxyindole in N,N-dimethylformamide (10 mL) was cooled to 0° C. and sodium hydride (0.20 g, 5 mmol, 60% dispersion in mineral oil) added. The mixture as stirred for 30 min and then warmed to room temperature. A solution of 2-bromoethyl acetate (0.50 g, 3 mmol) in N,N-dimethylformamide (2 mL) was added, and the mixture was stirred for 18 h at room temperature. The mixture was diluted with water (50 mL) and extracted with ethyl acetate (3×50 mL). The organic layer was dried over anhydrous magnesium sulfate, filtered and concentrated under reduced pressure. The residue was purified by flash column chromatography (silica gel, gradient: 100% hexanes to 50% ethyl acetate/hexanes) to afford 0.16 g (26%) of acetic acid 2-(6-benzylozyindol-1-yl)ethyl ester. MS: m/z 310.36 (M+1).

(ii) Acetic acid 2-(6-benzylozyindol-1-yl)ethyl ester (0.16 g, 0.517 mmol) was dissolved in tetrahydrofuran (50 mL) and treated with 20% palladium on carbon (0.1 g). The black suspension was shaken under an atmosphere of 50 psi hydrogen for 18 h, at which time it was filtered through a pad of celite and concentrated under reduced pressure to afford acetic acid 2-(6-hydroxy-indol-1-yl)-ethyl ester (92 mg, 81.1%). The compound was used without further purification. MS: m/z 205.20 (M+1).

(iii) (5R)-4-(4-[3-(2-Methoxybenzyloxy)-propoxy]-phenyl)-3-oxo-5-trifluoromethanesulfonyloxymethyl-piperazine-1-carboxylic acid tert-butyl ester (0.26 g, 0.41 mmol) was dissolved in acetonitrile (2 mL). Cesium carbonate (0.3 g, 0.8 mmol) and 2-(6-hydroxy-indol-1-yl)-ethyl ester (0.090 g, 0.41 mmol) were added, and the mixture was stirred for 1 h at room temperature. The reaction was concentrated under reduced pressure and the residue dissolved in ethyl acetate (15 mL). The organic layer was washed with water (2×10 mL), dried over anhydrous magnesium sulfate, filtered and concentrated under reduced pressure. The residue was purified by flash column chromatograpy (silica gel, gradient: 0% ethyl acetate/hexanes to 100% ethyl acetate) to afford (3R)-3-[1-(2-acetoxyethyl)-1H-indol-6-yloxymethyl]-4-(4-[3-methoxybenzyloxy)-propoxy]-phenyl)-5-oxopiperazine-1-carboxylic acid tert-butyl ester (202 mg, 70%). MS: m/z 702.81 (M+1).

(iv) (3R)-3-[1-(2-Acetoxyethyl)-1H-indol-6-yloxymethyl]-4-(4-[3-methoxybenzyloxy)-propoxy]-phenyl)-5-oxopiperazine-1-carboxylic acid tert-butyl ester (0.10 g, 0.142 mmol) was dissolved in methanol (2 mL) under nitrogen and cooled to 0° C. Acetyl chloride (0.12 g, 1.42 mmol) was added dropwise. The nitrogen inlet needle was removed and the reaction mixture was allowed to warm to room temperature over a period of 18 h. The reaction mixture was quenched with saturated sodium bicarbonate. The mixture was extracted with ethyl acetate (3×10 mL). The organic layer was dried over anhydrous magnesium sulfate, filtered and concentrated under reduced pressure. The resultant solid was purified by flash column chromatography (silica gel, gradient: 100% dichloromethane, then 95/5 dichloromethane/methanol to 90/10 dichloromethane/methanol) to afford 56 mg (65%) of acetic acid 2-[(2R)-6-(1-(4-[3-(2-methoxybenzyloxy)-propoxy]-phenyl)-6-oxopiperazin-2-ylmethoxy)-indol-1-yl]-ethyl ester. MS: m/z 602.69 (M+1).

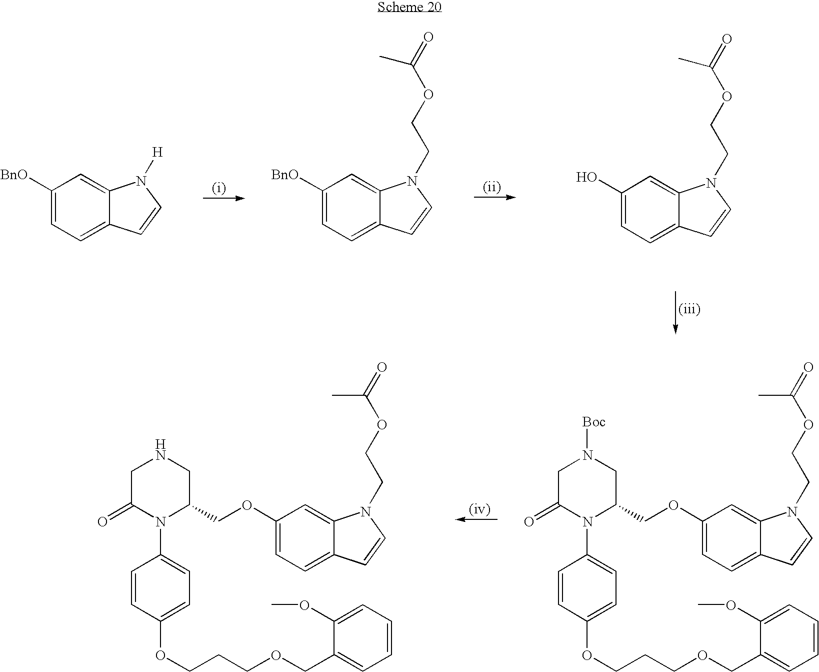

Method Q: Amidation of Aryl Halides (Scheme 21)

(i) An oven-dried, 10 mL round bottom flask was charged with 1-benzyloxy-4-iodobenzene (485 mg, 1.56 mmole), (3R)-3-hydroxymethyl-5-oxopiperazine-1-carboxylic acid tert-butyl ester (300 mg, 1.30 mmol), powered anhydrous potassium phosphate tribasic (553 mg, 2.61 mmole), copper(I) iodide (12 mg, 0.065 mmole). The flask was purged and back-filled with N₂. Anhydrous N,N-dimethylformamide (1.4 mL) was added, followed by N,N′-dimethyl-1,2-ethylenediamine (14 μL, 0.13 mmole). The purple suspension was heated in a 60° C. oil bath for 12 h. After cooling to room temperature, the white suspension was diluted with water and ethyl acetate. The layers were separated, and the aqueous layer was washed with ethyl acetate (2×). The combined organic layers were dried over MgSO₄, filtered, and concentrated under reduced pressure. The residue was purified by flash column chromatography (silica gel, gradient: 50% ethyl acetate/hexane to 70% ethyl acetate hexane) to provide 413 mg (77%) of (3R)-4-(4-benzyloxyphenyl)-3-hydroxymethyl-5-oxopiperazine-1-carboxylic acid tert-butyl ester as a white solid foam. MS: m/z 413.2 (M+1).

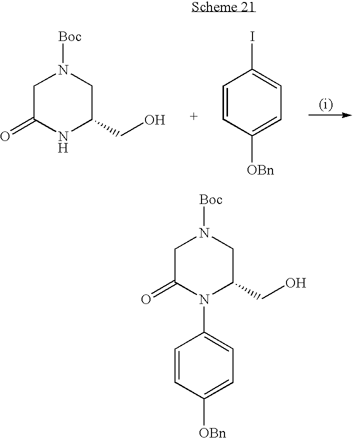

Method R: Preparation of Amide Analogs (Scheme 22)

(i) (3R)-3-Hydroxymethyl-5-oxopiperazine-1-carboxylic acid tert-butyl ester (500 mg, 2.17 mmol) and methyl 4-iodobenzoate (626 mg, 2.39 mmole) were reacted together as in Method Q. Purification by flash column chromatography (silica gel, gradient: 40% ethyl acetate/hexanes to 60% ethyl acetate/hexanes) gave 317 mg (40%) of (3R)-3-hydroxymethyl-4-(4-methoxycarbonylphenyl)-5-oxopiperazine-1-carboxylic acid tert-butyl ester as a clear viscous oil. MS: m/z 365.2 (M+1).

(ii) (3R)-3-Hydroxymethyl-4-(4-methoxycarbonylphenyl)-5-oxopiperazine-1-carboxylic acid tert-butyl ester (315 mg, 0.865 mmole) and 2-naphthol (187 mg, 1.30 mmole) were reacted together as in General Procedure 1. Purification by flash column chromatography (silica gel, gradient: 20% ethyl acetate/hexanes to 50% ethyl acetate/hexanes) gave 271 mg (64%) of (3R)-4-(4-methoxycarbonylphenyl)-3-(naphthalen-2-yloxymethyl)-5-oxopiperazine-1-carboxylic acid tert-butyl ester as a white solid. MS: m/z 491.2 (M+1).

(iii)(3R)-4-(4-Methoxycarbonylphenyl)-3-(naphthalen-2-yloxymethyl)-5-oxopiperazine-1-carboxylic acid tert-butyl ester (268 mg, 0.546 mmole) was dissolved in methanol (4 mL). A solution of 10% sodium hydroxide in water (1 mL) was added, and the reaction mixture was stirred at room temperature for 16 h. Tetrahydrofuran (4 mL) and water (4 mL) were added and the white suspension was stirred at room temperature for an additional 6 h. The clear solution was diluted with water and made acidic by the dropwise addition of 1 N hydrochloric acid. The aqueous layer was extracted with ethyl acetate (3×). The combined organics were washed with brine, dried over anhydrous magnesium sulfate, filtered, and concentrated under reduced pressure to yield 330 mg (100%) of (3R)-4-(4-carboxyphenyl)-3-(naphthalen-2-yloxymethyl)-5-oxopiperazine-1-carboxylic acid tert-butyl ester as a white solid that was used without further purification. MS: m/z 475.2 (M−1, AP−).

(iv)(3R)-4-(4-Carboxyphenyl)-3-(naphthalen-2-yloxymethyl)-5-oxopiperazine-1-carboxylic acid tert-butyl ester (325 mg, 0.546 mmole) was dissolved in anhydrous acetonitrile (3 mL) and anhydrous tetrahydrofuran (2 mL). Di-iso-propylethyl amine (0.3 mL), HBTU (285 mg, 0.75 mmole), and phenethylamine (103 μL, 0.818 mmole) were added sequentially, and the reaction mixture was stirred at room temperature for 25 h. The mixture was diluted with ethyl acetate and washed sequentially with a solution of 1:1 brine:aqueous 10% citric acid (2×), water, aqueous saturated sodium bicarbonate, and brine. The organic layer was dried over anhydrous magnesium sulfate, filtered, and concentrated under reduced pressure. Purification by flash column chromatography (silica gel, gradient: 40% ethyl acetate/hexanes to 80% ethyl acetate/hexanes) gave 219 mg (69%) of (3R)-3-(naphthalen-2-yloxymethyl)-5-oxo-4-(4-phenethylcarbamoylphenyl)-piperazine-1-carboxylic acid tert-butyl ester as a white solid. MS: m/z 580.2 (M+1).

(v) (3R)-3-(Naphthalen-2-yloxymethyl)-5-oxo-4-(4-phenethylcarbamoylphenyl)-piperazine-1-carboxylic acid tert-butyl ester (217 mg, 0.374 mmole) was treated with acetyl chloride (0.27 mL, 3.74 mmole) as in Method A. Purification by flash column chromatography (silica gel, gradient: 100% dichloromethane then 98/2 dichloromethane/methanol to 90/10 dichloromethane/methanol) afforded 127 mg (71%) of 4-[(2R)-2-(Naphthalen-2-yloxymethyl)-6-oxopiperazin-1-yl]-N-phenethylbenzamide as a white solid. MS: m/z 480.1 (M+1).

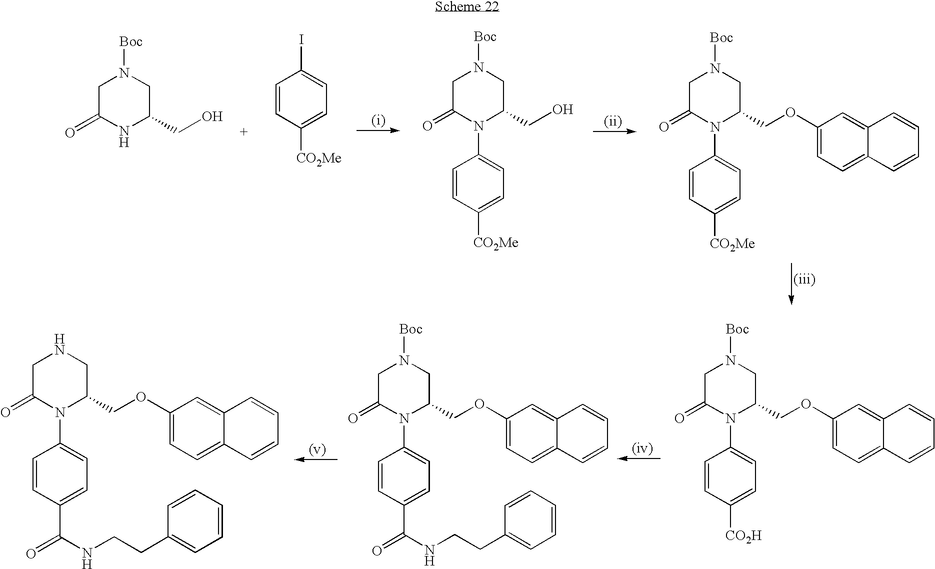

Method S: Preparation of (2R)-1-{4-[3-(4-Chlorobenzyloxy)-propoxy]-phenyl}-2-(4-methoxybenzyloxymethyl)-piperazine (Scheme 23)

(i) (3R)-4-(4-Hydroxyphenyl)-3-(4-methoxybenzyloxymethyl)-piperazine-1-carboxylic acid tert-butyl ester (892 mg, 2.082 mmol), was dissolved in anhydrous acetonitrile (8 mL) under a N₂ atmosphere. The solution was treated with 3-bromo-1-propanol (0.38 mL, 4.16 mmole) and potassium carbonate (432 mg, 3.12 mmole). The suspension was heated at reflux for 18 h. After cooling to room temperature, the suspension was filtered through a celite plug, and concentrated under reduced pressure. The crude residue was purified by flash column chromatography (silica gel, gradient: 40% ethyl acetate/hexanes to 60% ethyl acetate/hexanes) to yield 880 mg (87%) of (3R)-4-[4-(3-hydroxypropoxy)-phenyl]-3-(4-methoxybenzyloxymethyl)-piperazine-1-carboxylic acid tert-butyl ester as a white solid. MS: m/z 487.3 (M+1).

(ii) Sodium hydride (5 mg, 0.12 mmole, 60% suspension in mineral oil), was suspended in anhydrous N,N-dimethylformamide (0.3 mL). A solution of (3R)-4-[4-(3-hydroxypropoxy)-phenyl]-3-(4-methoxybenzyloxymethyl)-piperazine-1-carboxylic acid tert-butyl ester (50 mg, 0.10 mmol) in anhydrous N,N-dimethylformamide (0.3 mL) was added to the suspension, followed by 15-crown-5 (1 drop, catalytic). The suspension was stirred at room temperature for 1 hr. 4-Chlorobenzyl chloride (21 mg, 0.13 mmole) was added, and the mixture was heated in a 60° C. oil bath for 4 h. The mixture was concentrated under reduced pressure. Purification of the residue by flash column chromatography (silica gel, gradient: 10% ethyl acetate/hexanes to 30% ethyl acetate/hexanes) gave 34 mg (54%) of (3R)-4-{4-[3-(4-chlorobenzyloxy)-propoxy]-phenyl}-3-(4-methoxybenzyloxymethyl)-piperazine-1-carboxylic acid tert-butyl ester as a clear viscous oil

(iii) (3R)-4-{4-[3-(4-chlorobenzyloxy)-propoxy]-phenyl}-3-(4-methoxybenzyloxymethyl)-piperazine-1-carboxylic acid tert-butyl ester (34 mg, 0.06 mmole) was dissolved in anhydrous methanol (2 mL) and cooled in an ice bath. Acetyl chloride (39 μL, 0.56 mmol) was added dropwise. The nitrogen inlet needle was removed, and the reaction mixture allowed to warm to room temperature over a period of 18 h. The excess acid was quenched by addition of a solution of aqueous saturated sodium bicarbonate. The aqeous layer was extracted with ethyl acetate (3×). The combined organics were dried over anhydrous magnesium sulfate, filtered, and concentrated under reduced pressure. Purification by flash column chromatography (silica gel, gradient: 100:0 dichloromethane/methanol to 90/10 dichloromethane/methanol) provided 17 mg (58%) of (2R)-1-{4-[3-(4-chlorobenzyloxy)-propoxy]-phenyl}-2-(4-methoxybenzyloxymethyl)-piperazine as a semi-solid. MS: m/z 511.2 (M+1).

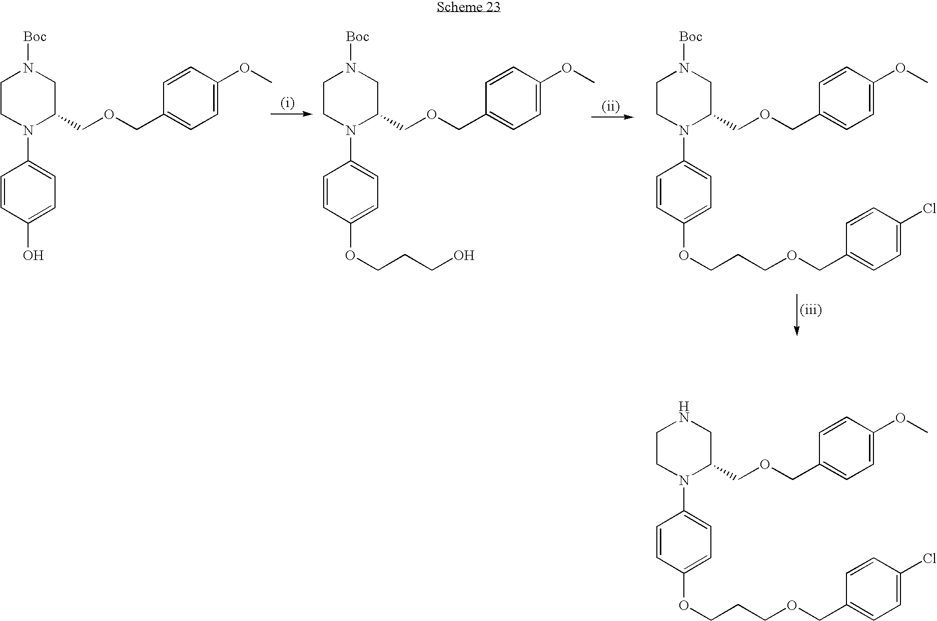

Method T: Formation of 2-Aza-Aryl Ethers (Scheme 24)

(i) (3R)-3-Hydroxymethyl-4-(4-[3-(2-methoxybenzyloxy)-propoxy]-phenyl)-5-oxopiperazine-1-carboxylic acid tert-butyl ester (0.200 g, 0.400 mmol), was dissolved in anhydrous tetrahydrofuran (3 mL) under a N₂ atmosphere. Sodium hydride (24 mg, 0.60 mmole) was added in a single portion. The resulting suspension was stirred at room temperature for 30 min. 2-Chloroquinoline (78 mg, 0.48 mmole) was added, and the reaction mixture was stirred at room temperature for 18 h. The solvent was removed under reduced pressure, and the residue was purified by flash column chromatography (silica gel, gradient: 40% ethyl acetate/hexanes to 70% ethyl acetate/hexanes) to afford 91 mg (36%) of (5R)-4-{4-[3-(2-methoxybenzyloxy)-propoxy]-phenyl}-3-oxo-5-(quinolin-2-yloxymethyl)-piperazine-1-carboxylic acid tert-butyl ester as a viscous oil. MS: m/z 628.3 (M+1).

(ii) (5R)-4-{4-[3-(2-Methoxybenzyloxy)-propoxy]-phenyl}-3-oxo-5-(quinolin-2-yloxymethyl)-piperazine-1-carboxylic acid tert-butyl ester (80 mg, 0.13 mmol) was dissolved in anhydrous methanol (3 mL) under nitrogen and cooled in an ice bath. Acetyl chloride (137 mg, 1.75 mmol) was added dropwise. The nitrogen inlet needle was removed, and the reaction mixture allowed to warm to room temperature over a period of 18 h. The mixture was concentrated under reduced pressure, dissolved in dichloromethane (1 mL), and treated with a solution of aqueous saturated sodium bicarbonate (0.5 mL). Purification by flash column chromatography (silica gel, gradient: 100:0 dichloromethane/methanol to 93/7 dichloromethane/methanol) provided 20 mg (22%) of (6R)-1-{4-[3-(2-methoxybenzyloxy)-propoxy]-phenyl}-6-(quinolin-2-yloxymethyl)-piperazin-2-one as a semi-solid. MS: m/z 528.6 (M+1).

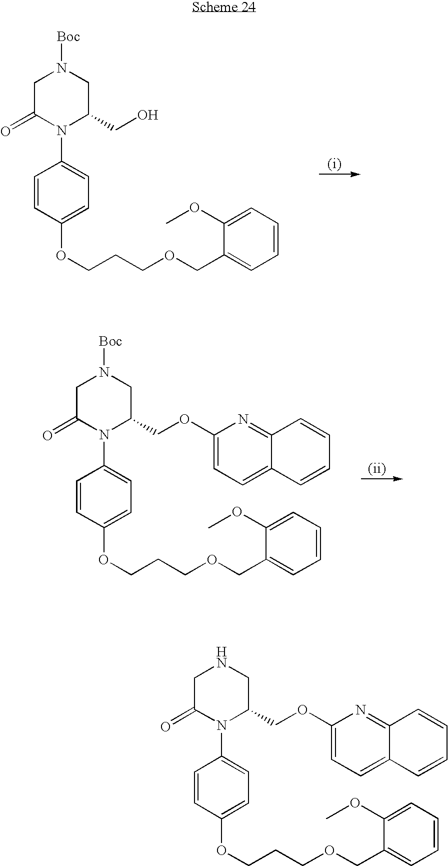

Method U: Reduction of Piperazinones to Piperazines (Scheme 25)

(i) (3R)-4-{4-[3-(2-Methoxybenzylsulfanyl)-propoxy]-phenyl}-3-(naphthalen-2-yloxymethyl)-5-oxopiperazine-1-carboxylic acid tert-butyl ester (280 mg, 0.44 mmol), was dissolved in anhydrous tetrahydrofuran (10 mL) under a N₂ atmosphere. Borane dimethylsulfide complex (0.12 mL, 1.31 mmole) was added, and the reaction mixture was heated in a 50° C. oil bath for 3 h. After cooling to room temperature, an aqueous solution of 10% sodium potassium tartrate (20 mL) was added dropwise. After gas evolution had ceased, the biphasic mixture was heated in a 50° C. oil bath for 18 h. After cooling to room temperature, the mixture was partitioned between water and ethyl acetate. The organic layer was washed with water, dried over anhydrous sodium sulfate, filtered, and concentrated under reduced pressure. Purification by flash column chromatography (silica gel, 50% ethyl acetate/hexanes) afforded 150 mg (55%) of (3R)-4-{4-[3-(2-methoxybenzylsulfanyl)-propoxy]-phenyl}-3-(naphthalen-2-yloxymethyl)-piperazine-1-carboxylic acid tert-butyl ester as a viscous oil. MS: m/z 629.3 (M+1).

(ii) (3R)-4-{4-[3-(2-Methoxybenzylsulfanyl)-propoxy]-phenyl}-3-(naphthalen-2-yloxymethyl)-piperazine-1-carboxylic acid tert-butyl ester (80 mg, 0.13 mmol) was dissolved in anhydrous dioxane (4 mL) under nitrogen and cooled in an ice bath. A solution of hydrogen chloride in dioxane (2.4 mL, 4.5 mmol) was added, and the reaction mixture was allowed to warm to room temperature over a period of 2 h. The mixture was treated with excess sodium bicarbonate, filtered, and concentrated under reduced pressure. Purification by flash column chromatography (silica gel, 98.5/2.5 dichloromethane/methanol) gave 50 mg (40%) of (2R)-1-{4-[3-(2-methoxybenzylsulfanyl)-propoxy]-phenyl}-2-(naphthalen-2-yloxymethyl)-piperazine as a viscous oil. MS: m/z 529.2 (M+1).

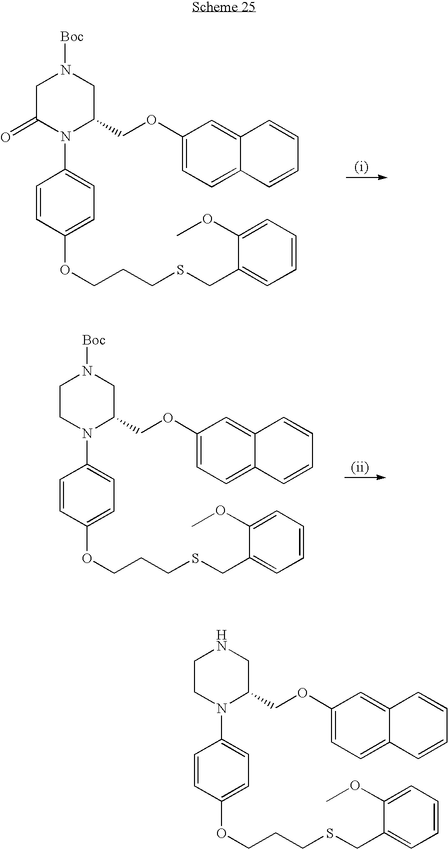

Method V: Preparation of (2R)-1-{4-[3-(2-Fluorobenzyloxy)-propoxy]-phenyl}-2-(naphthalen-2-yloxymethyl)-piperazine (Scheme 26)

(i) (3R)-4-(4-Benzyloxyphenyl)-3-(naphthalen-2-yloxymethyl)-piperazine-1-carboxylic acid tert-butyl ester (844 mg, 1.61 mmol) was dissolved in ethyl acetate (50 mL), and treated with 20% Pd(OH)₂/C (400 mg). The black suspension was shaken under a 50 psi H₂ atmosphere for 40 h. The suspension was filtered through a celite plug, and concentrated under reduced pressure. Purification by flash column chromatography (silica gel, gradient: 10% ethyl acetate/hexanes to 30% ethyl acetate/hexanes) yielded 401 mg (57%) of (3R)-4-(4-hydroxyphenyl)-3-(naphthalen-2-yloxymethyl)-piperazine-1-carboxylic acid tert-butyl ester as a white solid. MS: m/z 435.2 (M+1).

(ii) (3R)-4-(4-Hydroxyphenyl)-3-(naphthalen-2-yloxymethyl)-piperazine-1-carboxylic acid tert-butyl ester (844 mg, 1.61 mmol) was dissolved in acetonitrile (8 mL). Potassium carbonate (190 mg, 1.4 mmole) and 3-bromo-1-propanol (0.17 mL, 1.85 mmole) were added, and the resulting suspension heated to reflux for 18 h. The suspension was filtered through a celite plug, and concentrated under reduced pressure. Purification by flash column chromatography (silica gel, gradient: 25% ethyl acetate/hexanes to 50% ethyl acetate/hexanes) yielded 384 mg (57%) of (3R)-4-[4-(3-hydroxypropoxy)-phenyl]-3-(naphthalen-2-yloxymethyl)-piperazine-1-carboxylic acid tert-butyl ester as a white solid foam. MS: m/z 493.2 (M+1).

(iii)(3R)-4-[4-(3-Hydroxypropoxy)-phenyl]-3-(naphthalen-2-yloxymethyl)-piperazine-1-carboxylic acid tert-butyl ester was dissolved in anhydrous N,N-dimethylformamide (1 mL). Sodium hydride (15 mg, 0.37 mmole, 60% dispersion in mineral oil) and 15-crown-5 (1 drop, catalytic) were added, and the suspension was stirred at room temperature for 30 min. 2-Fluorobenzyl bromide (43 mg, 0.23 mmole) was added, and the suspension was stirred at room temperature overnight. The suspension was diluted with ethyl acetate and washed with water. The organic layer was dried over anhydrous magnesium sulfate, filtered, and concentrated under reduced pressure to afford (3R)-4-{4-[3-(2-fluorobenzyloxy)-propoxy]-phenyl}-3-(naphthalen-2-yloxymethyl)-piperazine-1-carboxylic acid tert-butyl ester a yellow oil that was used without further purification. MS: m/z 601.4 (M+1).

(iv) The crude (3R)-4-{4-[3-(2-fluorobenzyloxy)-propoxy]-phenyl}-3-(naphthalen-2-yloxymethyl)-piperazine-1-carboxylic acid tert-butyl ester obtained in step (iii) was dissolved in anhydrous methanol (5 m]L) and cooled in an ice bath. Acetyl chloride (143 mg, 1.83 mmole) was added dropwise, and the reaction mixture was stirred for 18 h, allowing the ice bath to warm to room temperature. The mixture was partitioned between dichloromethane and 2 N sodium carbonate. The organic layer was concentrated under reduced pressure. Purification of the residue by flash column chromatography (silica gel, gradient: 98:2 dichloromethane/methanol to 95/5 dichloromethane/methanol) provided 58 mg (63%) of (2R)-1-{4-[3-(2-fluorobenzyloxy)-propoxy]-phenyl}-2-(naphthalen-2-yloxymethyl)-piperazine as a clear glass. MS: m/z 501.3 (M+1).

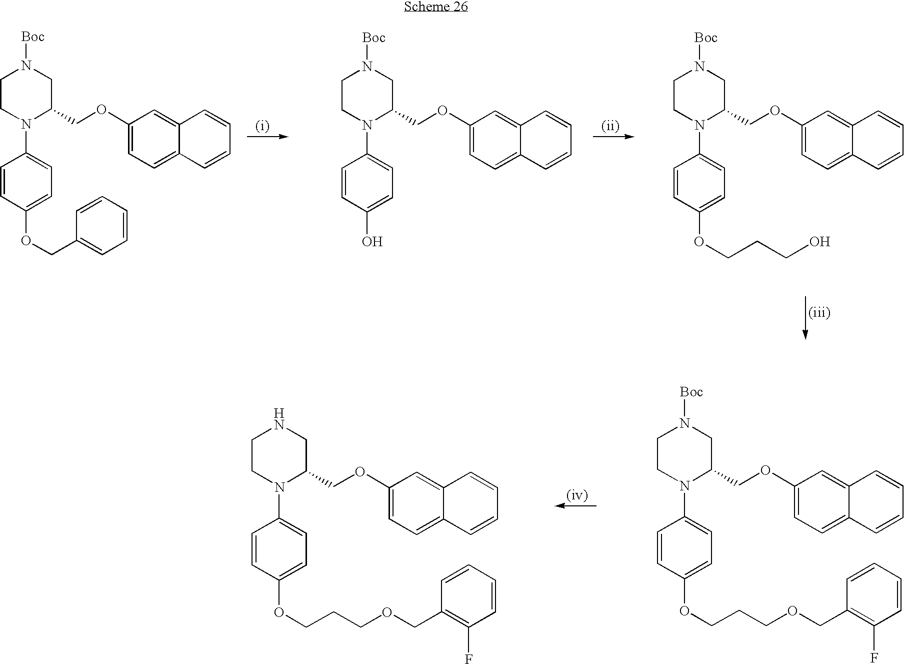

Method W: Preparation of (3R)-3-Hydroxymethyl-4-(4-hydroxyphenyl)-piperazine-1-carboxylic acid tert-butyl ester (Scheme 27)

(i) (2R)-4-Benzyl-1-(4-benzyloxyphenyl)-2-(4-methoxybenzyloxymethyl)-piperazine (2.5 g, 4.9 mmole) was dissolved in a solution of 50:50 trifluoroacetic acid:dichloromethane, and stirred at room temperature for 4 h. The solution was partitioned between water and ethyl acetate. The organic layer was washed with saturated sodium bicarbonate (3×) and water (2×), dried over anhydrous magnesium sulfate, filtered, and concentrated. The residue was purified by flash column chromatography (silica gel, 100% ethyl acetate) to yield 2.20 g (100%) of [4-benzyl-1-(4-benzyloxyphenyl)-piperazin-2-yl]-methanol. MS: m/z 389 (M+1).

(ii) A solution of [4-Benzyl-1-(4-benzyloxyphenyl)-piperazin-2-yl]-methanol (2.2 g, 5.7 mmole) in tetrahydrofuran (50 mL) and ethanol (50 mL) was treated with 20% Pd/C (0.5 g). The resulting black suspension was shaken under a 50 psi H₂ atmosphere for 48 h, with an additional 0.5 g and 1.0 g of 20% Pd/C added at 16 h and 43 h, respectively. The mixture was filtered through celite and concentrated under reduced pressure. The residue was dissolved in tetrahydrofuran and methanol, treated with di-tert-butyldicarbonate (1.65 g, 7.5 mmole), and stirred at room temperature for 18 h. The solution was concentrated under reduced pressure and purified by flash column chromatography (silica gel, gradient: 0:100 ethyl acetate:hexane to 80:20 ethyl acetate:hexane) to provide 802 mg (53%) of 3-hydroxymethyl-4-(4-hydroxyphenyl)-piperazine-1-carboxylic acid tert-butyl ester. MS: m/z 309.1 (M+1).

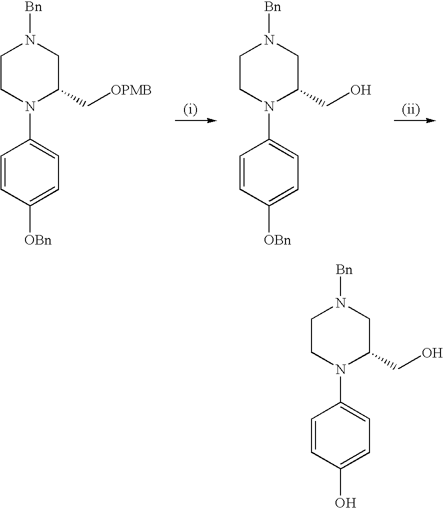

### EXAMPLES

### Example 1

Synthesis of (6S)-1-{4-[3-(2-methoxybenzyloxy)-propoxy]-phenyl}-6-(naphthalen-2-yloxymethyl)-piperazin-2-one

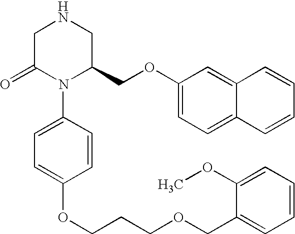

The compound of Example 1 was prepared according to methods A and B, utilizing intermediates with an S configuration. MS: m/z 527.2 (M+1).

### Example 2

Synthesis of (6R)-6-(3,4-dichlorobenzyloxymethyl)-1-{4-[3-(2-methoxybenzyloxy)-propoxy]-phenyl}-piperazin-2-one

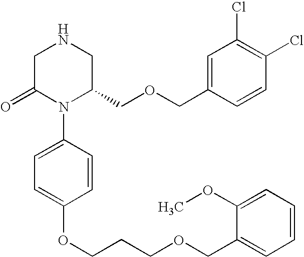

The compound of Example 2 was prepared according to methods A and C utilizing 3,4-dichlorobenzyl bromide instead of benzyl bromide. MS: m/z 561.1 (M+1).

### Example 3

Synthesis of (6R)-6-(2-fluorobenzyloxymethyl)-1-{4-[3-(2-methoxybenzyloxy)-propoxy]-phenyl}-piperazin-2-one

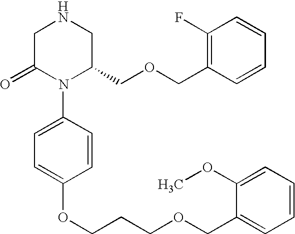

The compound of Example 3 was prepared according to methods A and C utilizing 2-fluorobenzyl bromide instead of benzyl bromide. MS: m/z 509.2 (M+1).

### Example 4

Synthesis of (6R)-6-(3,4-difluorobenzyloxymethyl)-1-{4-[3-(2-methoxybenzyloxy)-propoxy]-phenyl}-piperazin-2-one

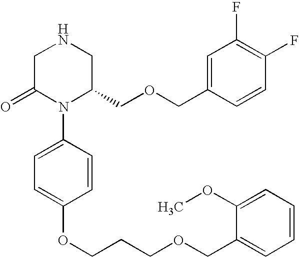

The compound of Example 4 was prepared according to methods A and C utilizing 3,4-difluorobenzyl bromide instead of benzyl bromide. MS: m/z 527.1 (M+1).

### Example 5

Synthesis of (6R)-6-(4-chlorobenzyloxymethyl)-1-{4-[3-(2-methoxybenzyloxy)-propoxy]-phenyl}-piperazin-2-one

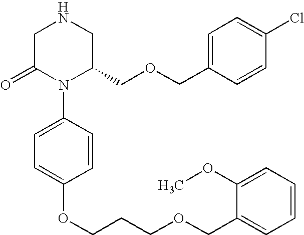

The compound of Example 5 was prepared according to methods A and C utilizing 3,4-dichlorobenzyl bromide instead of benzyl bromide. MS: m/z 527.1 (M+1).

### Example 6

Synthesis of (6R)-6-(3-chlorobenzyloxymethyl)-1-{4-[3-(2-methoxybenzyloxy)-propoxy]-phenyl}-piperazin-2-one

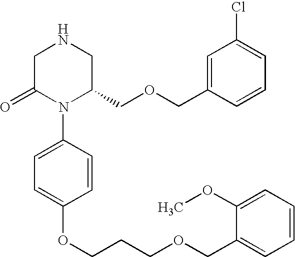

The compound of Example 6 was prepared according to methods A and C utilizing 3-chlorobenzyl bromide instead of benzyl bromide. MS: m/z 527.1 (M+1).

### Example 7

Synthesis of (6R)-1-{4-[3-(2-methoxybenzyloxy)-propoxy]-phenyl}-6-(4-methylbenzyloxymethyl)-piperazin-2-one

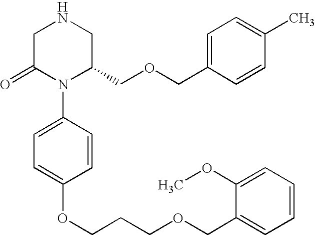

The compound of Example 7 was prepared according to methods A and C utilizing 4-methoxybenzyl chloride instead of benzyl bromide. MS: m/z 505.2 (M+1).

### Example 8

Synthesis of (6R)-6-(4-fluorobenzyloxymethyl)-1-{4-[3-(2-methoxybenzyloxy)-propoxy]-phenyl}-piperazin-2-one

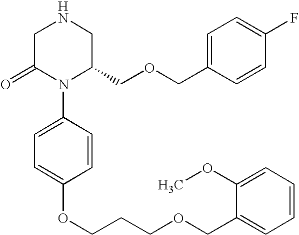

The compound of Example 8 was prepared according to methods A and C utilizing 4-fluorobenzyl bromide instead of benzyl bromide. MS: m/z 527 (M+1).

### Example 9

Synthesis of (6R)-6-(3-methoxybenzyloxymethyl)-1-{4-[3-(2-methoxybenzyloxy)-propoxy]-phenyl}-piperazin-2-one

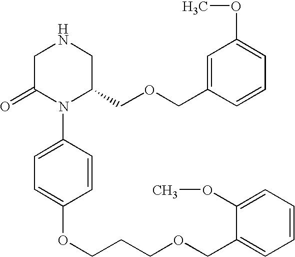

The compound of Example 9 was prepared according to methods A and C utilizing 3-methoxybenzyl bromide instead of benzyl bromide. MS: m/z 521.2 (M+1).

### Example 10

Synthesis of (6R)-6-(2-methoxybenzyloxymethyl)-1-{4-[3-(2-methoxybenzyloxy)-propoxy]-phenyl}-piperazin-2-one

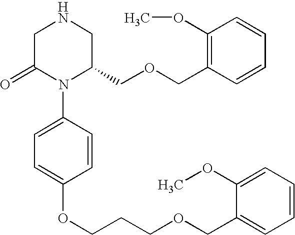

The compound of Example 10 was prepared according to methods A and C utilizing 2-methoxybenzyl chloride instead of benzyl bromide. MS: m/z 521.2 (M+1).

### Example 11

Synthesis of (6R)-6-(3,5-difluorobenzyloxymethyl)-1-{4-[3-(2-methoxybenzyloxy)-propoxy]-phenyl}-piperazin-2-one

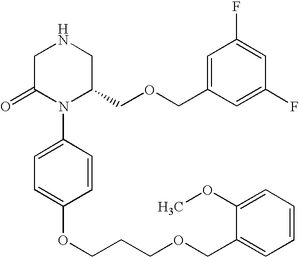

The compound of Example 11 was prepared according to methods A and C utilizing 3,5-difluorobenzyl bromide instead of benzyl bromide. MS: m/z 527.2 (M+1).

### Example 12

Synthesis of (6R)-6-(4-methoxybenzyloxymethyl)-1-{4-[3-(2-methoxybenzyloxy)-propoxy]-phenyl}-piperazin-2-one

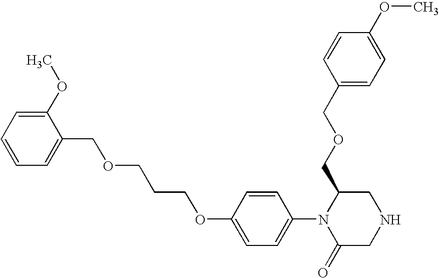

The compound of Example 12 was prepared according to methods A and C utilizing 4-methoxybenzyl chloride instead of benzyl bromide. MS: m/z 521.2 (M+1)

### Example 13

Synthesis of (6R)-1-{4-[3-(2-methoxybenzyloxy)-propoxy]-phenyl}-6-(4-trifluoromethylbenzyloxymethyl)-piperazin-2-one

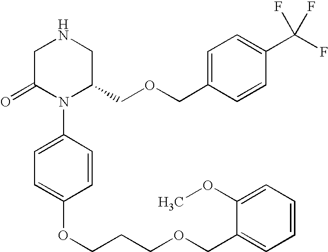

The compound of Example 13 was prepared according to methods A and C utilizing 4-trifluoromethylbenzyl bromide instead of benzyl bromide. MS: m/z 559.1 (M+1).

### Example 14

Synthesis of (6R)-6-(2-chlorobenzyloxymethyl)-1-{4-[3-(2-methoxybenzyloxy)-propoxy]-phenyl}-piperazin-2-one

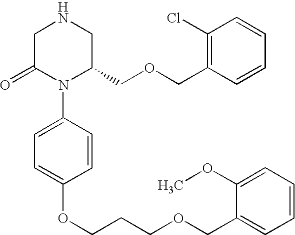

The compound of Example 14 was prepared according to methods A and C utilizing 2-chlorobenzyl bromide instead of benzyl bromide. MS: m/z 527.1 (M+1).

### Example 15

Synthesis of (6R)-1-{4-[3-(2-methoxybenzyloxy)-propoxy]-phenyl}-6-(3-methylbenzyloxymethyl)-piperazin-2-one

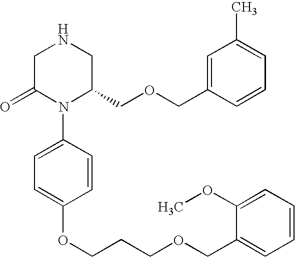

The compound of Example 15 was prepared according to methods A and C utilizing 3-methylbenzyl bromide instead of benzyl bromide. MS: m/z 559.1 (M+1).

### Example 16

Synthesis of (6R)-6-(2,6-difluorobenzyloxymethyl)-1-{4-[3-(2-methoxybenzyloxy)-propoxy]-phenyl}-piperazin-2-one

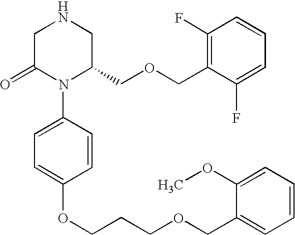

The compound of Example 16 was prepared according to methods A and C utilizing 3,6-difluorobenzyl bromide instead of benzyl bromide. MS: m/z 527.2 (M+1).

### Example 17

Synthesis of (6R)-6-(2,6-dichlorobenzyloxymethyl)-1-{4-[3-(2-methoxybenzyloxy)-propoxy]-phenyl}-piperazin-2-one

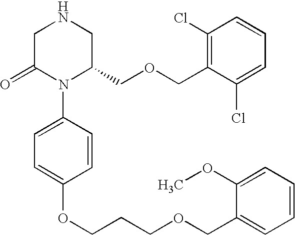

The compound of Example 17 was prepared according to methods A and C utilizing 2,6-dichlorobenzyl chloride instead of benzyl bromide. MS: m/z 559.1 (M+1).

### Example 18

Synthesis of (6R)-6-(3-fluorobenzyloxymethyl)-1-{4-[3-(2-methoxybenzyloxy)-propoxy]-phenyl}-piperazin-2-one

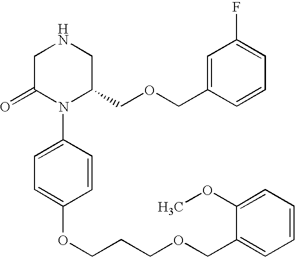

The compound of Example 18 was prepared according to methods A and C utilizing 3-fluorobenzyl bromide instead of benzyl bromide. MS: m/z 509.2 (M+1).

### Example 19

Synthesis of (6R)-6-(4-fluoro-2-trifluoromethylbenzyloxymethyl)-1-{4-[3-(2-methoxybenzyloxy)-propoxy]-phenyl}-piperazin-2-one

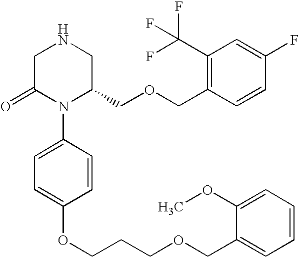

The compound of Example 19 was prepared according to methods A and C utilizing 4-fluoro-2-trifluoromethylbenzyl bromide instead of benzyl bromide. MS: m/z 577.2 (M+1).

### Example 20

Synthesis of (6R)-6-(3,5-dichlorobenzyloxymethyl)-1-{4-[3-(2-methoxybenzyloxy)-propoxy]-phenyl}-piperazin-2-one

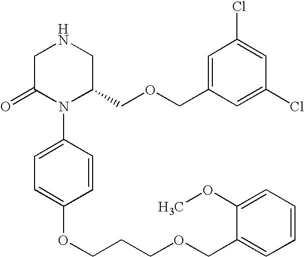

The compound of Example 20 was prepared according to methods A and C utilizing 3,5-dichlorobenzyl chloride instead of benzyl bromide. MS: m/z 559.1 (M+1).

### Example 21

Synthesis of (6R)-1-{4-[3-(2-methoxybenzyloxy)-propoxy]-phenyl}-6-(2-methylbenzyloxymethyl)-piperazin-2-one

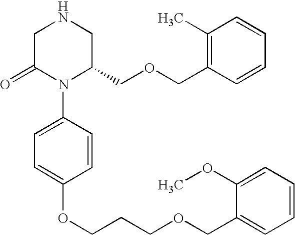

The compound of Example 21 was prepared according to methods A and C utilizing 2-methylbenzyl bromide instead of benzyl bromide. MS: m/z 505.2 (M+1).

### Example 22

Synthesis of (6R)-6-(2-chloro-4-fluorobenzyloxymethyl)-1-{4-[3-(2-methoxybenzyloxy)-propoxy]-phenyl}-piperazin-2-one

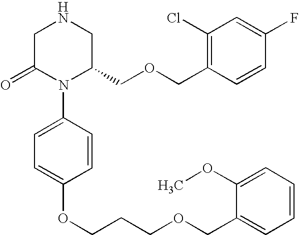

The compound of Example 22 was prepared according to methods A and C utilizing 2-chloro-4-fluorobenzyl bromide instead of benzyl bromide. MS: m/z 543.1 (M+1).

### Example 23

Synthesis of (6R)-1-{4-[3-(2-methoxybenzyloxy)-propoxy]-phenyl}-6-(pyridin-3-ylmethoxymethyl)-piperazin-2-one

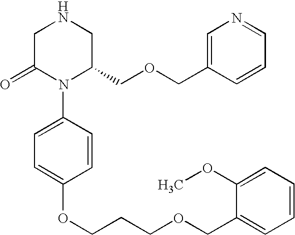

The compound of Example 23 was prepared according to methods A and C utilizing 3-chloromethylpyridine hydrochloride instead of benzyl bromide. MS: m/z 492.2 (M+1).

### Example 24

Synthesis of (6R)-6-(4-chloro-3-trifluoromethylbenzyloxymethyl)-1-{4-[3-(2-methoxybenzyloxy)-propoxy]-phenyl}-piperazin-2-one

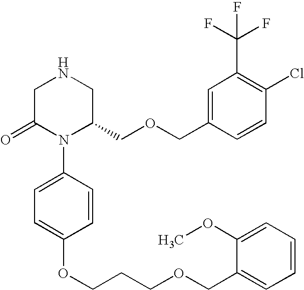

The compound of Example 24 was prepared according to methods A and C utilizing 4-chloro-3-trifluoromethylbenzyl bromide instead of benzyl bromide. MS: m/z 595.1 (M+1).

### Example 25

Synthesis of (6R)-1-{4-[3-(2-methoxybenzyloxy)-propoxy]-phenyl}-6-(pyridin-4-ylmethoxymethyl)-piperazin-2-one

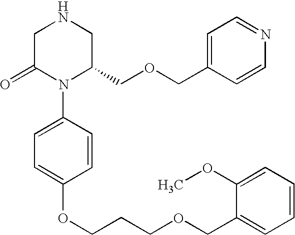

The compound of Example 25 was prepared according to methods A and C utilizing 4-chloromethylpyridine hydrochloride instead of benzyl bromide. MS: m/z 492.2 (M+1).

### Example 26

Synthesis of (6R)-6-(4-fluoro-3-trifluoromethylbenzyloxymethyl)-1-{4-[3-(2-methoxybenzyloxy)-propoxy]-phenyl}-piperazin-2-one

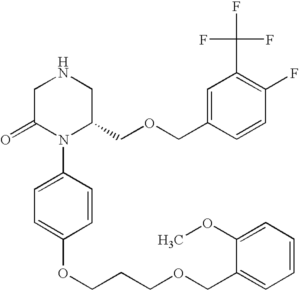

The compound of Example 26 was prepared according to methods A and C utilizing 4-fluoro-3-trifluoromethylbenzyl bromide instead of benzyl bromide. MS: m/z 577.1 (M+1).

### Example 27

Synthesis of (6R)-6-(4-fluoro-3-methylbenzyloxymethyl)-1-{4-[3-(2-methoxybenzyloxy)-propoxy]-phenyl}-piperazin-2-one

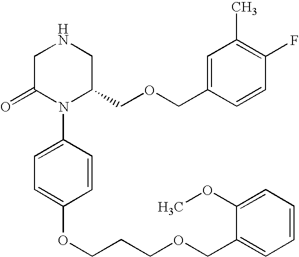

The compound of Example 27 was prepared according to methods A and C utilizing 4-fluoro-3-methylbenzyl bromide instead of benzyl bromide. MS: m/z 523.2 (M+1).

### Example 28

Synthesis of (6R)-4-(1-{4-[3-(2-methoxybenzyloxy)-propoxy]-phenyl}-6-oxopiperazin-2-ylmethoxymethyl)-benzonitrile

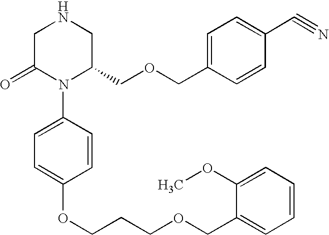

The compound of Example 28 was prepared according to methods A and C utilizing 4-cyanobenzyl bromide instead of benzyl bromide. MS: m/z 516.2 (M+1).

### Example 29

Synthesis of (6R)-1-{4-[3-(2-methoxybenzyloxy)-propoxy]-phenyl}-6-(pyridin-2-ylmethoxymethyl)-piperazin-2-one

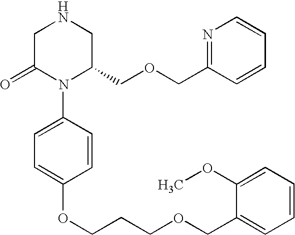

The compound of Example 29 was prepared according to methods A and C utilizing 2-chloromethylpyridine hydrochloride instead of benzyl bromide. MS: m/z 492.2 (M+1).

### Example 30

Synthesis of (6R)-6-(4-bromobenzyloxymethyl)-1-{4-[3-(2-methoxybenzyloxy)-propoxy]-phenyl}-piperazin-2-one

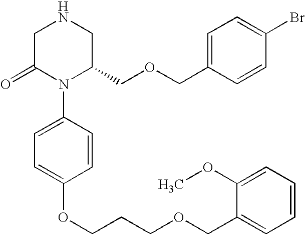

The compound of Example 30 was prepared according to methods A and C utilizing 4-bromobenzyl bromide instead of benzyl bromide. MS: m/z 571.1 (M+1).

### Example 31

Synthesis of acetic acid 2-[7-(1-{4-[3-(2-methoxybenzyloxy)-propoxy]-phenyl}-6-oxopiperazin-(2R)-ylmethoxy)-3,4-dihydro-2H-quinolin-1-yl]-ethyl ester

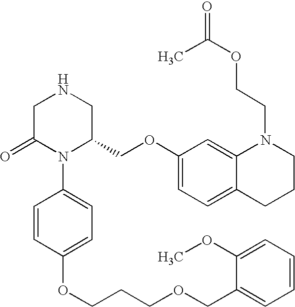

The compound of Example 31 was prepared according to methods A, B and O utilizing 7-hydroxyquinoline instead of 2-naphthol. MS: m/z 618.3 (M+1).

### Example 32

Synthesis of (2R)-1-{4-[3-(2-methoxybenzyloxy)-propoxy]-phenyl}-2-(naphthalen-2-yloxymethyl)-piperazine

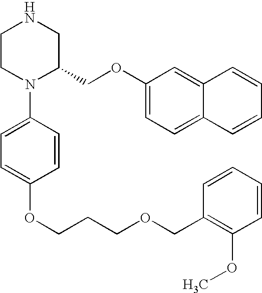

The compound of Example 32 was prepared according to methods F, I and G utilizing 1-93-iodopropoxymethyl)-2-methoxybenzene instead of 4-benzyloxy-1-bromobenzene. MS: m/z 513.3 (M+1).

### Example 33

Synthesis of (2R)-2-(4-methoxybenzyloxymethyl)-1-{4-[3-(2-methoxy-benzyloxy)-propoxy]-phenyl}-piperazine

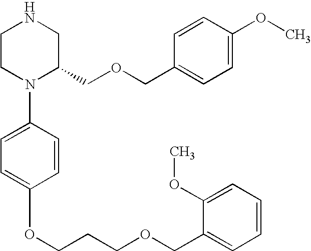

The compound of Example 33 was prepared according to method H and step (vi) of method A. MS: m/z 507.3 (M+1)

### Example 34

Synthesis of (2R)-1-[4-(3-benzyloxypropoxy)-phenyl]-2-(4-methoxybenzyloxymethyl)-piperazine

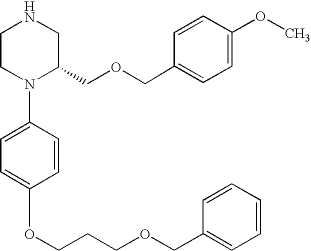

The compound of Example 34 was prepared according to method H and step (vi) of method A utilizing 3-iodopropoxymethylbenzene instead of 1-(3-iodopropoxymethyl)-2-methoxybenzene. MS: m/z 477.3 (M+1)

### Example 35

Synthesis of (2R)-1-(4-benzyloxyphenyl)-2-(naphthalen-2-yloxymethyl)-piperazine

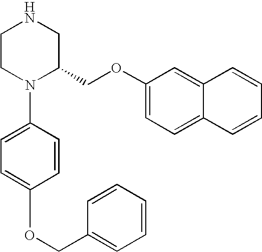

The compound of Example 35 was prepared according to methods F, I and G utilizing 4-benzyloxy-1-bromobenzene instead of 1-bromo-4-(3-(2-methoxybenzyloxy)-propyloxy)-benzene. MS: m/z 454.2 (M+1)

### Example 36

Synthesis of (2R)-1-(4-benzyloxyphenyl)-2-(4-methoxybenzyloxymethyl)-piperazine

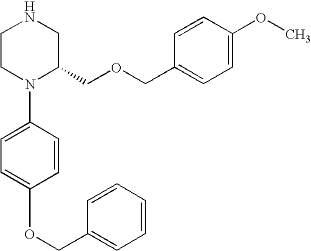

The compound of Example 36 was prepared according to method H and step (ii) of method B. MS: m/z 419.2 (M+1)

### Example 37

Synthesis of (6R)-1-{4-[3-(2-methoxybenzyloxy)-propoxy]-phenyl}-6-(naphthalen-2-yloxymethyl)-piperazin-2-one

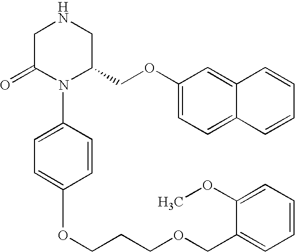

The compound of Example 37 was prepared according to methods A and B. MS: m/z 527.2 (M+1)

### Example 38

Synthesis of (2R)-1-[4-(3-benzyloxypropoxy)-phenyl]-2-(naphthalen-2-yloxymethyl)-piperazine

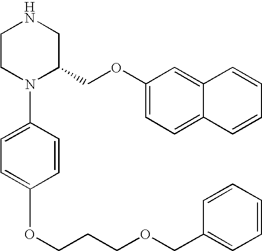

The compound of Example 38 was prepared according to methods F, I, G, utilizing 4-benzyloxy-1-bromobenzene instead of 1-bromo-4-(3-(2-methoxybenzyloxy)-propyloxy)-benzene, step (vi) of method H, step (vi) of method A, utilizing 3-iodopropoxymethylbenzene instead of 1-(3-iodopropoxymethyl)-2-methoxybenzene, and step (ii) of method B. MS: m/z 483.3 (M+1)

### Example 39

Synthesis of (6R)-1-{3-fluoro-4-[3-(2-methoxybenzyloxy)-propoxy]-phenyl}-6-(naphthalen-2-yloxymethyl)-piperazin-2-one

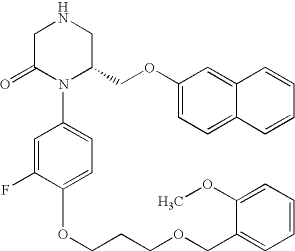

The compound of Example 39 was prepared according to methods F, Q, utilizing 4-bromo-2-fluoro-1-[3-(2-methoxybenzyloxy)-propoxy]-benzene instead of 1-benzyloxy-4-iodobenzene, and B. MS: m/z 595.1 (M+1)

### Example 40

Synthesis of (2R)-1-{4-[3-(2-methoxybenzyloxy)-propoxy]-phenyl}-2-(5,6,7,8-tetrahydronaphthalen-2-yloxymethyl)-piperazine

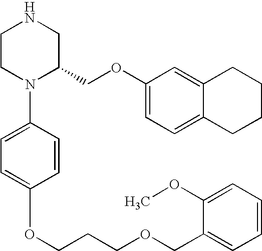

The compound of Example 40 was prepared according to methods F, I, steps (v-vi) of method H, step (vi) of A, and step (ii) of method B. Step (vi) of method H afforded the above-identified compound as a side product. MS: m/z 517.3 (M+1)

### Example 41

Synthesis of (6R)-1-{4-[3-(2-methoxybenzyloxy)-propoxy]-phenyl}-6-(quinolin-7-yloxymethyl)-piperazin-2-one

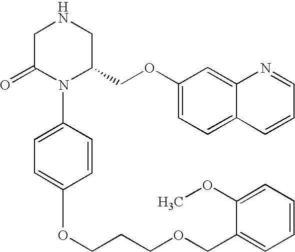

The compound of Example 41 was prepared according to methods A and B utilizing 7-hydroxy quinoline instead of 2-naphthol. MS: m/z 528.2 (M+1)

### Example 42

Synthesis of (6R)-1-{4-[3-(2-methoxybenzyloxy)-propoxy]-phenyl}-6-(1,2,3,4-tetrahydroquinolin-7-yloxymethyl)-piperazin-2-one

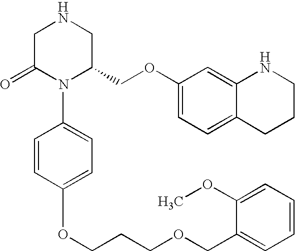

The compound of Example 42 was prepared according to methods A and B utilizing 7-hydroxy quinoline instead of 2-naphthol. The quinoline moiety was then reduced to the tetrahydroquinoline as in step (i) of method 0. MS: m/z 532.2 (M+1)

### Example 43

Synthesis of (6R)-1-{3,5-difluoro-4-[3-(2-methoxybenzyloxy)-propoxy]-phenyl}-6-(naphthalen-2-yloxymethyl)-piperazin-2-one

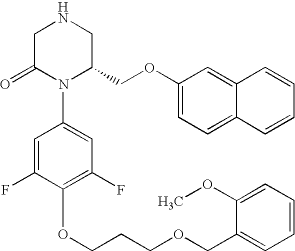

The compound of Example 43 was prepared according to methods F, Q, and B, utilizing 4-bromo-2,6-difluoro-1-[3-(2-methoxybenzyloxy)-propoxy]-benzene instead of 1-benzyloxy-4-iodobenzene. MS: m/z 563.2 (M+1)

### Example 44

Synthesis of (6R)-6-[1-(3-hydroxypropyl)-1,2,3,4-tetrahydroquinolin-7-yloxymethyl]-1-{4-[3-(2-methoxybenzyloxy)-propoxy]-phenyl}-piperazin-2-one

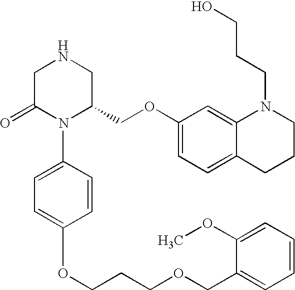

The compound of Example 44 was prepared according to methods A, B and O utilizing 3-bromopropanol instead of 2-bromoethanol. MS: m/z 590.3 (M+1)

### Example 45

Synthesis of (6R)-6-benzyloxymethyl-1-{4-[3-(2-methoxybenzyloxy)-propoxy]-phenyl}-piperazin-2-one

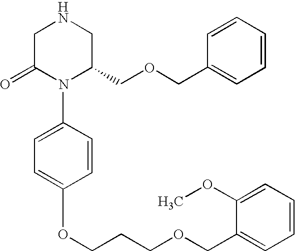

The compound of Example 45 was prepared according to methods A and C. MS: m/z 491.2 (M+1)

### Example 46

Synthesis of (6S)-6-(4-fluorobenzyloxymethyl)-1-{4-[3-(2-methoxybenzyloxy)-propoxy]-phenyl}-piperazin-2-one

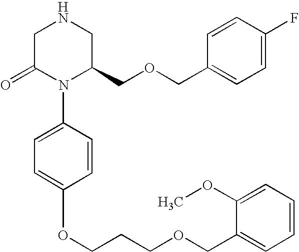

The compound of Example 46 was prepared according to methods A and C utilizing intermediates with an S configuration and 4-fluorobenzyl bromide instead of benzyl bromide. MS: m/z 561.1 (M+1).

### Example 47

Synthesis of 4-[(2R)-2-(naphthalen-2-yloxymethyl)-6-oxopiperazin-1-yl]-N-phenethylbenzamide

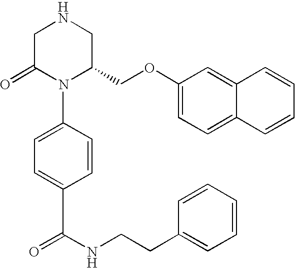

The compound of Example 47 was prepared according to method R. MS: m/z 480.1 (M+1).

### Example 48

Synthesis of (6R)-1-[4-methoxy-3-(3-methoxypropoxy)-phenyl]-6-(naphthalen-2-yloxymethyl)-piperazin-2-one

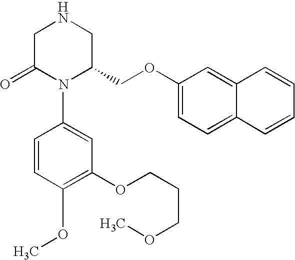

The compound of Example 48 was prepared according to methods F, Q, and B, utilizing 4-bromo-1-methody-2-(3-methoxypropoxy)-benzene instead of 1-benzyloxy-4-iodobenzene. MS: m/z 451.2 (M+1).

### Example 49

Synthesis of N-(2-ethoxyethyl)-4-[(2R)-2-(naphthalen-2-yloxymethyl)-6-oxopiperazin-1-yl]-benzamide

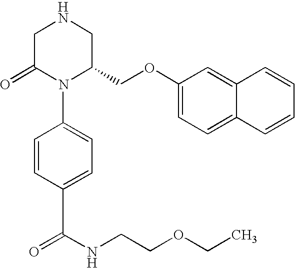

The compound of Example 49 was prepared according to method R utilizing 2-ethoxy-ethylamine instead of phenethylamine. MS: m/z 448.1 (M+1).

### Example 50

Synthesis of N-[2-(3-methoxyphenyl)-ethyl]-4-[(2R)-2-(naphthalen-2-yloxymethyl)-6-oxopiperazin-1-yl]-benzamide

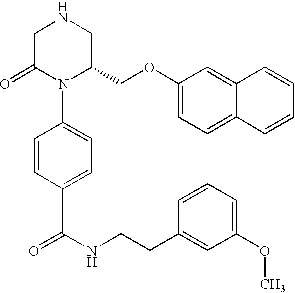

The compound of Example 50 was prepared according to method R utilizing 2-(3-methoxyphenyl)-ethylamine instead of phenethylamine. MS: m/z 510.2 (M+1).

### Example 51

Synthesis of (6R)-6-(isoquinolin-7-yloxymethyl)-1-{4-[3-(2-methoxybenzyloxy)-propoxy]-phenyl}-piperazin-2-one

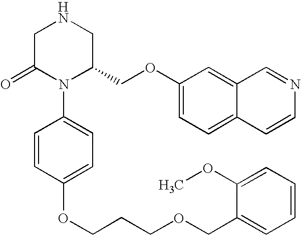

The compound of Example 51 was prepared according to methods A and B utilizing 7-hydroxyisoquinoline instead of 2-naphthol. MS: m/z 528.2 (M+1)

### Example 52

Synthesis of (6R)-1-{4-[3-(2-methoxybenzyloxy)-propoxy]-phenyl}-6-(quinolin-6-yloxymethyl)-piperazin-2-one

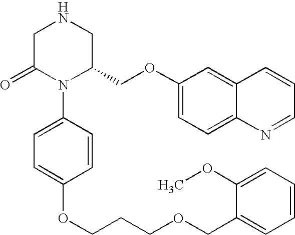

The compound of Example 52 was prepared according to methods A and B utilizing 6-hydroxy quinoline instead of 2-naphthol. MS: m/z 528.2 (M+1)

### Example 53

Synthesis of 4-[(2R)-2-(naphthalen-2-yloxymethyl)-6-oxopiperazin-1-yl]-N-(2-phenoxyethyl)-benzamide

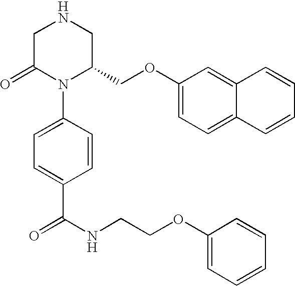

The compound of Example 53 was prepared according to method R utilizing 2-phenoxyethylamine instead of phenethylamine. MS: m/z 496.2 (M+1).

### Example 54

Synthesis of (6R)-6-(1-acetyl-1,2,3,4-tetrahydroquinolin-6-yloxymethyl)-1-{4-[3-(2-methoxybenzyloxy)-propoxy]-phenyl}-piperazin-2-one

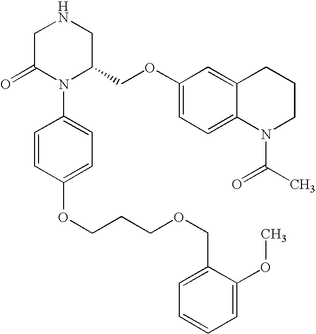

The compound of Example 54 was prepared according to methods A and B utilizing 6-hydroxyquinoline instead of 2-naphthol. The quinoline moiety was then reduced as in method Q. Deprotection as in step (ii) of method B gave the above-identified compound as a side product. MS: m/z 573.28 (M+1).

### Example 55

Synthesis of (6R)-1-{4-[3-(2-methoxybenzyloxy)-propoxy]-phenyl}-6-(1-thiazol-4-ylmethyl-1,2,3,4-tetrahydroquinolin-7-yloxymethyl)-piperazin-2-one

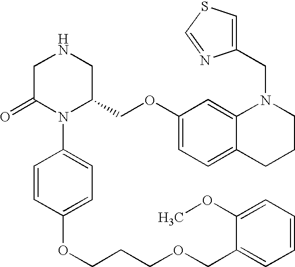

The compound of Example 55 was prepared according to methods A and B utilizing 7-hydroxyquinoline instead of 2-naphthol. The quinoline moiety was then reduced and alkylated as in method O utilizing 4-(chloromethyl)thiazole hydrochloride instead of 2-bromoethanol. MS: m/z 629.28 (M+1).

### Example 56

Synthesis of 2-[7-(1-{4-[3-(2-methoxybenzyloxy)-propoxy]-phenyl}-6-oxopiperazin-(2R)-ylmethoxy)-3,4-dihydro-2H-quinolin-1-yl]-acetamide

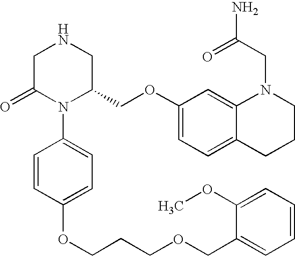

The compound of Example 56 was prepared according to methods A and B utilizing 7-hydroxyquinoline instead of 2-naphthol. The quinoline moiety was then reduced and alkylated as in method 0 utilizing 2-chloroacetamide instead of 2-bromoethanol. MS: m/z 629.28 (M+1).

### Example 57

Synthesis of (6R)-6-[1-(2-hydroxyethyl)-1,2,3,4-tetrahydroquinolin-7-yloxymethyl]-1-{4-[3-(2-methoxybenzyloxy)-propoxy]-phenyl}-piperazin-2-one

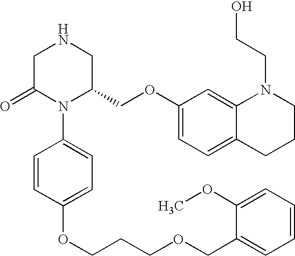

The compound of Example 57 was prepared according to methods A, B and O. MS: m/z 618.3 (M+1).

### Example 58

Synthesis of naphthalene-2-carboxylic acid (2R)-1-{4-[3-(2-methoxybenzyloxy)-propoxy]-phenyl}-6-oxopiperazin-2-yl methyl ester

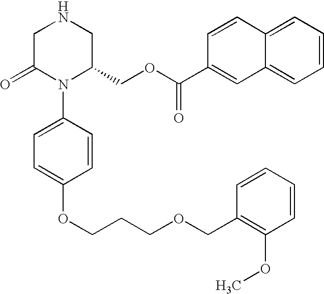

The compound of Example 58 was prepared according to methods A and D. MS: m/z 555.63 (M+1).

### Example 59

Synthesis of 4-methyl-benzoic acid (2R)-1-{4-[3-(2-methoxybenzyloxy)-propoxy]-phenyl}-6-oxopiperazin-2-yl methyl ester

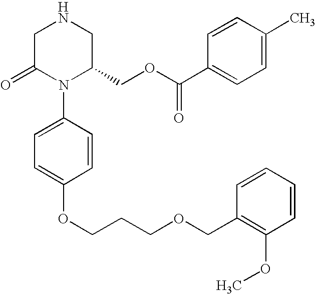

The compound of Example 59 was prepared according to methods A and D utilizing 4-methoxybenzoyl chloride instead of 2-naphthoyl chloride. MS: m/z 519.6 (M+1).

### Example 60

Synthesis of 4-chloro-benzoic acid (2R)-1-{4-[3-(2-methoxybenzyloxy)-propoxy]-phenyl}-6-oxopiperazin-2-yl methyl ester

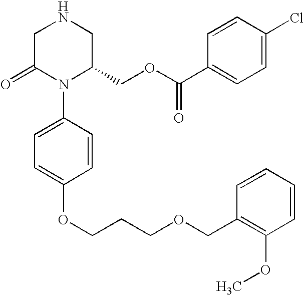

The compound of Example 60 was prepared according to methods A and D utilizing 4-chlorobenzoyl chloride instead of 2-naphthoyl chloride. MS: m/z 540.02 (M+1).

### Example 61

Synthesis of benzoic acid (2R)-1-{4-[3-(2-methoxybenzyloxy)-propoxy]-phenyl}-6-oxopiperazin-2-yl methyl ester

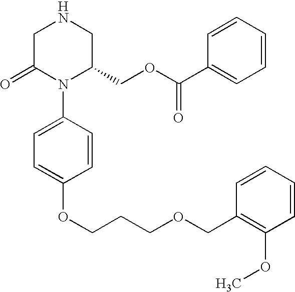

The compound of Example 61 was prepared according to methods A and D utilizing benzoyl chloride instead of 2-naphthoyl chloride. MS: m/z 505.57 (M+1).

### Example 62

Synthesis of (2R)-1-{4-[3-(4-chlorobenzyloxy)-propoxy]-phenyl}-2-(4-methoxybenzyloxymethyl)-piperazine

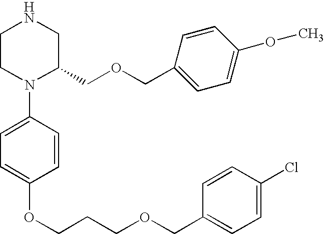

The compound of Example 62 was prepared according to method S. MS: m/z 511.23 (M+1)

### Example 63

Synthesis of (2R)-1-{4-[3-(3,4-dichlorobenzyloxy)-propoxy]-phenyl}-2-(4-methoxybenzyloxymethyl)-piperazine

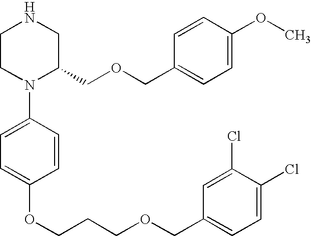

The compound of Example 63 was prepared according to method S utilizing 3,4-dichlorobenzyl bromide instead of 4-chlorobenzyl chloride. MS: m/z 545.19 (M+1)

### Example 64

Synthesis of (2R)-1-{4-[3-(3-chlorobenzyloxy)-propoxy]-phenyl}-2-(4-methoxybenzyloxymethyl)-piperazine

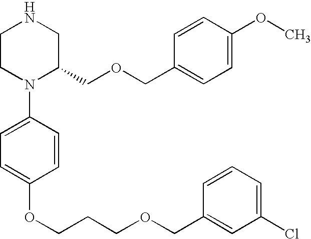

The compound of Example 64 was prepared according to method S utilizing 3-chlorobenzyl bromide instead of 4-chlorobenzyl chloride. MS: m/z 511.23 (M+1)

### Example 65

Synthesis of (2R)-2-(4-methoxybenzyloxymethyl)-1-{4-[3-(4-methoxybenzyloxy)-propoxy]-phenyl}-piperazine

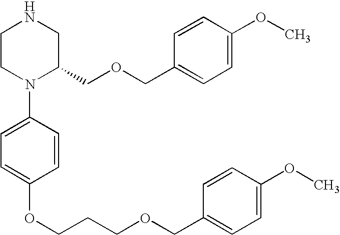

The compound of Example 65 was prepared according to method S utilizing 4-methoxybenzyl chloride instead of 4-chlorobenzyl chloride. MS: m/z 507.28 (M+1)

### Example 66

Synthesis of (2R)-1-{4-[3-(2-chlorobenzyloxy)-propoxy]-phenyl}-2-(4-methoxybenzyloxymethyl)-piperazine

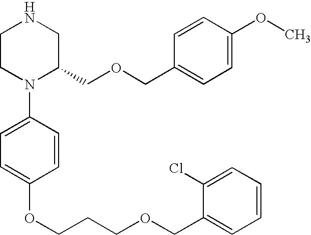

The compound of Example 66 was prepared according to method S utilizing 2-chlorobenzyl bromide instead of 4-chlorobenzyl chloride. MS: m/z 511.23 (M+1)

### Example 67

Synthesis of (2R)-1-{4-[3-(3,5-difluorobenzyloxy)-propoxy]-phenyl}-2-(4-methoxybenzyloxymethyl)-piperazine

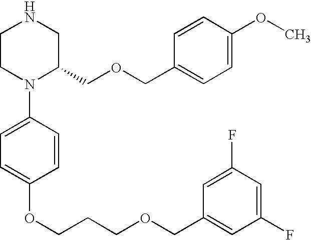

The compound of Example 67 was prepared according to method S utilizing 3,5-difluorobenzyl bromide instead of 4-chlorobenzyl chloride. MS: m/z 513.25 (M+1)

### Example 68

Synthesis of (2R)-2-(4-methoxybenzyloxymethyl)-1-{4-[3-(4-methylbenzyloxy)-propoxy]-phenyl}-piperazine

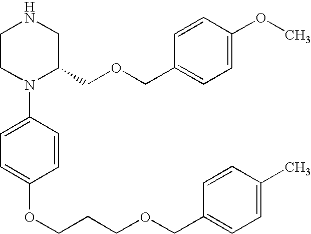

The compound of Example 68 was prepared according to method S utilizing 4-methylbenzyl bromide instead of 4-chlorobenzyl chloride. MS: m/z 491.28 (M+1)

### Example 69

Synthesis of (2R)-2-(4-methoxybenzyloxymethyl)-1-{4-[3-(3-methoxybenzyloxy)-propoxy]-phenyl}-piperazine

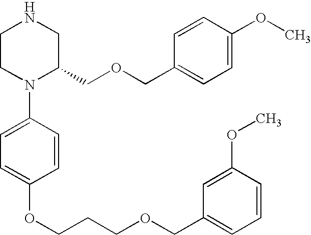

The compound of Example 69 was prepared according to method S utilizing 3-methoxybenzyl chloride instead of 4-chlorobenzyl chloride. MS: m/z 507.28 (M+1)

### Example 70

Synthesis of (2R)-2-(4-methoxybenzyloxymethyl)-1-{4-[3-(2-methoxyphenoxy)-propoxymethyl]-phenyl}-piperazine

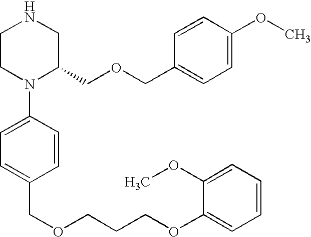

The compound of Example 70 was prepared according to method H, G and step (vi) of method A utilizing 4-benzyloxymethyl-1-bromobenzene instead of 4-benzyloxy-1-bromobenzene and 1-(3-iodopropoxy)-2-methoxybenzene instead of 1-(3-iodopropoxymethyl)-2-methoxybenzene. MS: m/z 507.28 (M+1)

### Example 71

Synthesis of (6R)-6-(4-fluorobenzyloxymethyl)-1-{4-[4-(2-methoxyphenoxy)-butoxy]-phenyl}-piperazin-2-one

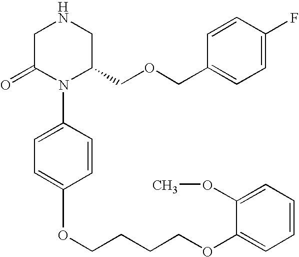

The compound of Example 71 was prepared according to methods A and C utilizing 4-fluorobenzyl bromide instead of benzyl bromide and 1-(4-iodobutoxy)-2-methoxybenzene instead of 1-(3-iodopropoxymethyl)-2-methoxybenzene. MS: m/z 509.24 (M+1)

### Example 72

Synthesis of (2R)-1-{4-[2-(2-methoxybenzyloxy)-ethoxymethyl]-phenyl}-2-(4-methoxybenzyloxymethyl)-piperazine

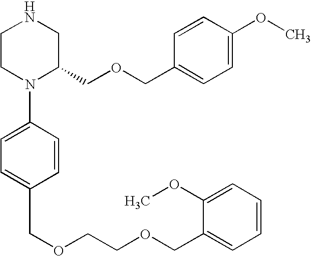

The compound of Example 72 was prepared according to method H, utilizing 4-benzyloxymethyl-1-bromobenzene instead of 4-benzyloxy-1-bromobenzene, step (vi) of method A, utilizing 1-(2-iodoethoxymethyl)-2-methoxybenzene instead of 1-(3-iodopropxymethyl)-1-methoxy benzene, and step (ii) of method B. MS: m/z 507.28 (M+1)

### Example 73

Synthesis of (6R)-1-{4-[4-(2-methoxyphenoxy)-butoxy]-phenyl}-6-(naphthalen-2-yloxymethyl)-piperazin-2-one

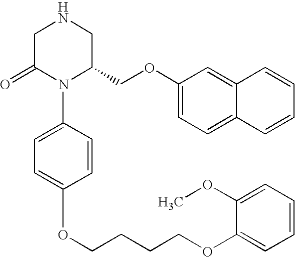

The compound of Example 73 was prepared according to methods A and B utilizing 1-(4-iodobutoxy)-2-methoxybenzene instead of 1-(3-iodopropoxymethyl)-2-methoxybenzene. MS: m/z 527.25 (M+1)

### Example 74

Synthesis of (6R)-6-(4-fluorobenzyloxymethyl)-1-{4-[2-(2-methoxybenzyloxy)-ethoxymethyl]-phenyl}-piperazin-2-one

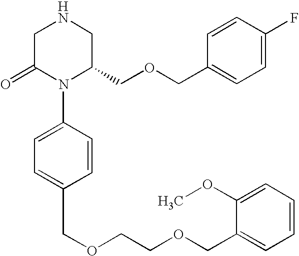

The compound of Example 74 was prepared according to methods A and C utilizing 4-fluorobenzyl bromide instead of benzyl bromide, 4-benzyloxymethyl-1-bromobenzene instead of 4-benzyloxy-1-bromobenzene, and 1-(2-iodoethoxymethyl)-2-methoxybenzene instead of 1-(3-iodopropoxymethyl)-2-methoxybenzene. MS: m/z 509.24 (M+1)

### Example 75

Synthesis of (2R)-2-(4-methoxybenzyloxymethyl)-1-{4-[4-(2-methoxyphenoxy)-butoxy]-phenyl}-piperazine

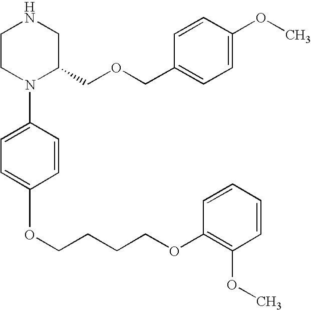

The compound of Example 75 was prepared according to method H and step (vi) of method A utilizing 1-(4-iodobutoxy)-2-methoxybenzene instead of 1-(3-iodopropoxymethyl)-2-methoxybenzene. MS: m/z 507.28 (M+1)

### Example 76

Synthesis of (6R)-6-(4-fluorobenzyloxymethyl)-1-{4-[3-(2-methoxyphenoxy)-propoxymethyl]-phenyl}-piperazin-2-one

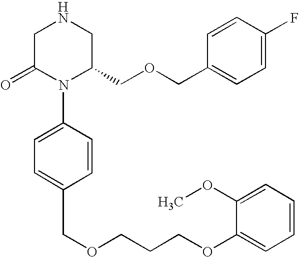

The compound of Example 76 was prepared according to methods A and C utilizing 4-fluorobenzyl bromide instead of benzyl bromide, 4-benzyloxymethyl-1-bromobenzene instead of 4-benzyloxy-1-bromobenzene, and 1-(3-iodopropoxy)-2-methoxybenzene instead of 1-(3-iodopropoxymethyl)-2-methoxybenzene. MS: m/z 509.24 (M+1)

### Example 77

Synthesis of (6R)-1-{4-[3-(2-methoxybenzyloxy)-propoxy]-phenyl}-6-(quinolin-2-yloxymethyl)-piperazin-2-one

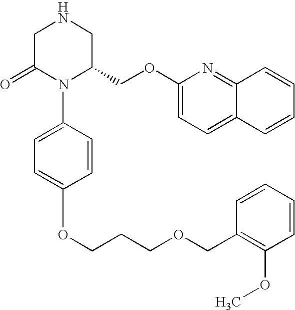

The compound of Example 77 was prepared according to methods A and T. MS: m/z 528.61 (M+1)

### Example 78

Synthesis of (6R)-1-{4-[3-(2-methoxybenzyloxy)-propoxy]-phenyl}-6-(quinoxalin-2-yloxymethyl)-piperazin-2-one

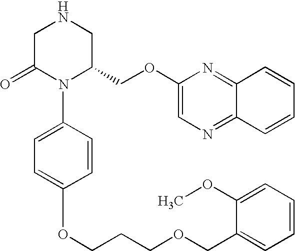

The compound of Example 78 was prepared according to methods A and T. utilizing 2-chloroquinoxaline instead of 2-chloroquinoline. MS: m/z 529.6 (M+1)

### Example 79

Synthesis of (6R)-1-{4-[3-(2-methoxybenzyloxy)-propoxy]-phenyl}-6-(pyrazin-2-yloxymethyl)-piperazin-2-one

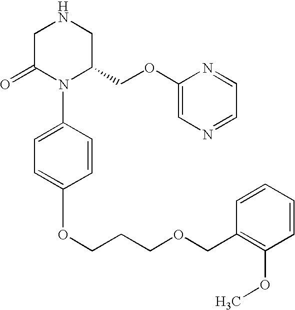

The compound of Example 79 was prepared according to methods A and T. utilizing 2-chloropyrazine instead of 2-chloroquinoline. MS: m/z 479.54 (M+1)

### Example 80

Synthesis of (6R)-1-{4-[3-(2-methoxybenzyloxy)-propoxy]-phenyl}-6-(pyridin-2-yloxymethyl)-piperazin-2-one

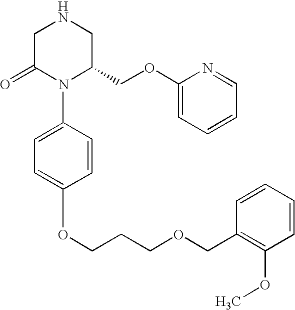

The compound of Example 80 was prepared according to methods A and T. utilizing 2-chloropyridine instead of 2-chloroquinoline. MS: m/z 478.55 (M+1)

### Example 81

Synthesis of (6R)-1-{4-[3-(2-methoxybenzyloxy)-propoxy]-phenyl}-6-(pyrimidin-2-yloxymethyl)-piperazin-2-one

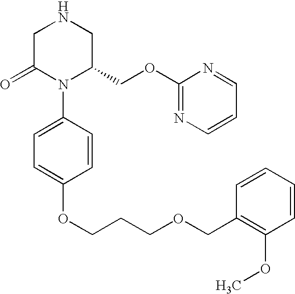

The compound of Example 81 was prepared according to methods A and T. utilizing 2-chloropyrimidine instead of 2-chloroquinoline. MS: m/z 479.54 (M+1)

### Example 82

Synthesis of 2-methoxy-N-([2R]-1-{4-[3-(2-methoxybenzyloxy)-propoxy]-phenyl}-6-oxopiperazin-2-ylmethyl)-benzamide

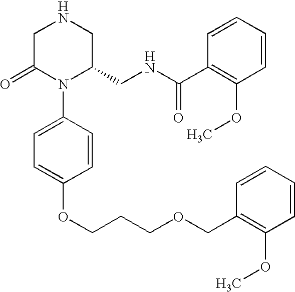

The compound of Example 82 was prepared according to methods A, K and L. MS: m/z 534.62 (M+1)

### Example 83

Synthesis of 4-chloro-N-([2R]-1-{4-[3-(2-methoxybenzyloxy)-propoxy]-phenyl}-6-oxopiperazin-2-ylmethyl)-benzamide

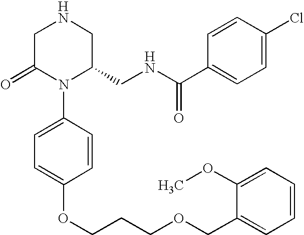

The compound of Example 83 was prepared according to methods A, K and L utilizing 4-chlorobenzoyl chloride instead of 2-methoxybenzoyl chloride. MS: m/z 539.03 (M+1)

### Example 84

Synthesis of N-([2R]-1-{4-[3-(2-methoxy-benzyloxy)-propoxy]-phenyl}-6-oxopiperazin-2-ylmethyl)-benzamide

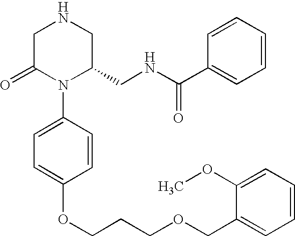

The compound of Example 84 was prepared according to methods A, K and L utilizing benzoyl chloride instead of 2-methoxybenzoyl chloride. MS: m/z 504.59 (M+1)

### Example 85

Synthesis of naphthalene-2-carboxylic acid ([2R]-1-{4-[3-(2-methoxybenzyloxy)-propoxy]-phenyl}-6-oxopiperazin-2-ylmethyl)-amide

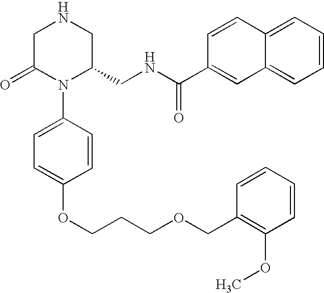

The compound of Example 85 was prepared according to methods A, K and L utilizing 2-naphthoyl chloride instead of 2-methoxybenzoyl chloride. MS: m/z 554.65 (M+1)

### Example 86

Synthesis of 2-fluoro-N-([2R]-1-{4-[3-(2-methoxybenzyloxy)-propoxy]-phenyl}-6-oxopiperazin-2-ylmethyl)-benzamide

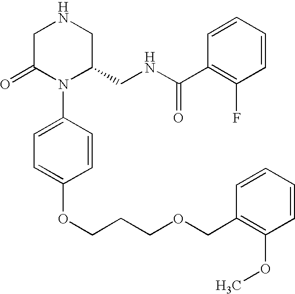

The compound of Example 86 was prepared according to methods A, K and L utilizing 2-fluorobenzoyl chloride instead of 2-methoxybenzoyl chloride. MS: m/z 522.58 (M+1)

### Example 87

Synthesis of (2R)-1-{4-[3-(2-fluorobenzyloxy)-propoxy]-phenyl}-2-(naphthalen-2-yloxymethyl)-piperazine

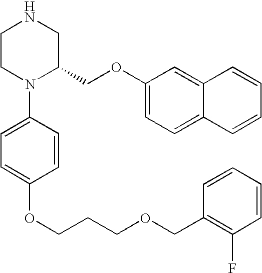

The compound of Example 87 was prepared according to methods F, I, step (v) of G, utilizing 2-methoxy-1-[2-(4-bromobenzyloxy)-ethoxy]methylbenzene instead of 4-benzyloxymethyl-1-bromobenzene, and step (ii) of B. MS: m/z 501.6 (M+1)

### Example 88

Synthesis of (2R)-1-{4-[3-(2-ethoxybenzyloxy)-propoxy]-phenyl}-2-(naphthalen-2-yloxymethyl)-piperazine

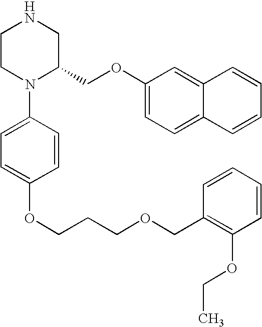

The compound of Example 88 was prepared according to method V utilizing 2-ethoxybenzyl bromide instead of 2-fluorobenzyl bromide. MS: m/z 527.67 (M+1)

### Example 89

Synthesis of (2R)-1-{4-[3-(3-methoxybenzyloxy)-propoxy]-phenyl}-2-(naphthalen-2-yloxymethyl)-piperazine

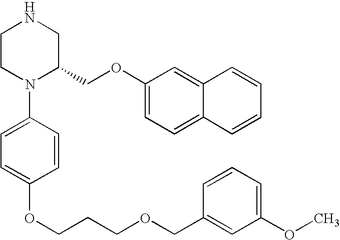

The compound of Example 89 was prepared according to method V utilizing 3-methoxybenzyl bromide instead of 2-fluorobenzyl bromide. MS: m/z 513.64 (M+1)

### Example 90

Synthesis of (2R)-1-{4-[3-(2-methoxybenzyloxy)-propoxy]-phenyl}-2-(naphthalen-2-ylmethoxymethyl)-piperazine

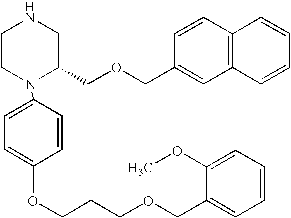

The compound of Example 90 was prepared according to methods W, step (vi) of A, and C utilizing 2-bromomethylnaphthalene instead of benzyl bromide. MS: m/z 527.28 (M+1)

### Example 91

Synthesis of (2R)-2-(biphenyl-3-ylmethoxymethyl)-1-{4-[3-(2-methoxybenzyloxy)-propoxy]-phenyl}-piperazine

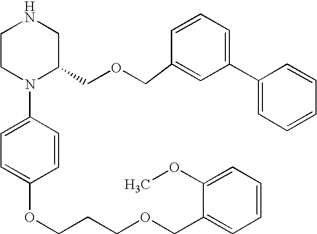

The compound of Example 91 was prepared according to methods W, step (vi) of A, and C utilizing b-bromomethylbiphenyl instead of benzyl bromide. MS: m/z 553.7 (M+1)

### Example 92

Synthesis of (6R)-6-(biphenyl-4-yloxymethyl)-1-(4-[3-(2-methoxybenzyloxy)-propoxy]-phenyl)-piperazin-2-one

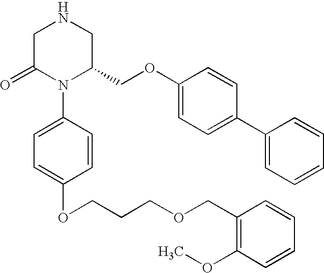

The compound of Example 92 was prepared according to methods A and B utilizing biphenyl-4-ol instead of 2-naphthol. MS: m/z 553.1 (M+1)

### Example 93

Synthesis of N-[4-([2R]-1-{4-[3-(2-methoxybenzyloxy)-propoxy]-phenyl}-6-oxopiperazin-2-ylmethoxy)-phenyl]-acetamide

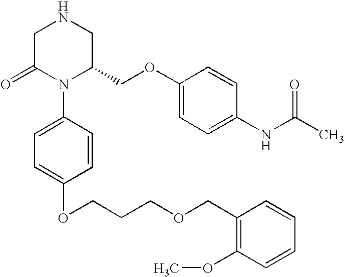

The compound of Example 93 was prepared according to methods A and B utilizing N-(4-hydroxy-phenyl)-acetamide instead of 2-naphthol. MS: m/z 534 (M+1)

### Example 94

Synthesis of (2R)-1-{4-[2-(2-methoxybenzyloxy)-ethoxymethyl]-phenyl}-2-(naphthalene-2-yloxymethyl)-piperazine

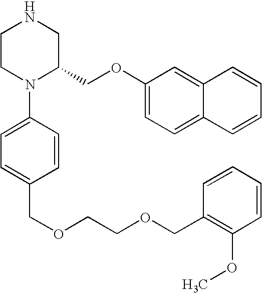

The compound of Example 87 was prepared according to methods F, I and G utilizing 4-benzyloxymethyl-1-bromobenzene instead of 4-benzyloxy-1-bromobenzene, and 1-(2-iodoethoxymethyl)-2-methoxybenzene instead of 1-(3-iodopropoxymethyl)-2-methoxybenzene. MS: m/z 513 (M+1)

### Example 95

Synthesis of 4-([2R]-1-{4-[3-(2-methoxybenzyloxy)-propoxy]-phenyl}-6-oxopiperazin-2-ylmethoxy)-N,N-dimethyl-benzamide

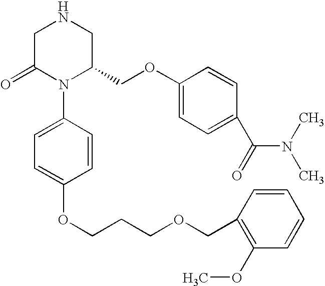

The compound of Example 95 was prepared according to methods A and B utilizing 4-hydroxy-N,N-dimethyl-benzamide instead of 2-naphthol. MS: m/z 548 (M+1)

### Example 96

Synthesis of 2-[(5R)-3-(2-methoxybenzyloxy)-propoxy]-5-[2-(naphthalene-2-yloxymethyl)-6-oxopiperazin-1-yl]-benzoic acid methyl ester

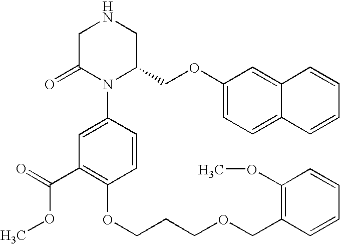

The compound of Example 96 was prepared according to methods F, Q, utilizing 5-iodo-2-[3-(2-methoxybenzyloxy)-propoxy]-benzoic acid methyl ester instead of 1-benzyloxy-4-iodobenzene, and B. MS: m/z 585 (M+1)

### Example 97

Synthesis of (2R)-1-(4-[3-(2-methoxyphenoxy)-propoxymethyl]-phenyl)-2-(naphthalene-2-yloxymethyl)-piperazine

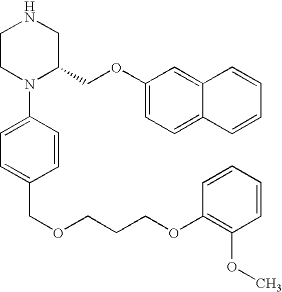

The compound of Example 97 was prepared according to methods F, I, step (v) of G, utilizing 2-methoxy-1-[3-(4-bromobenzyloxy)-propoxy]-benzene instead of 4-benzyloxymethyl-1-bromobenzene, and step (ii) of B. MS: m/z 513 (M+1)

### Example 98

Synthesis of 2-[(5R)-3-(2-methoxybenzyloxy)-propoxy]-5-[2-(naphthalene-2-yloxymethyl)-6-oxopiperazin-1-yl]-benzoic acid

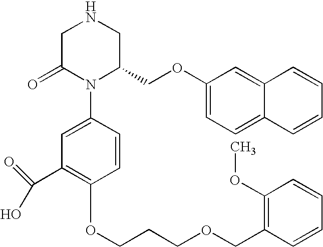

The compound of Example 98 was prepared as in Example 96 followed by hydrolysis of the methyl ester to afford the above-identified compound. MS: m/z 571 (M+1)

### Example 99

Synthesis of (2R)-1-(4-methoxymethylphenyl)-2-(naphthalene-2-yloxymethyl)-piperazine

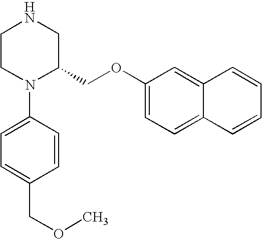

The compound of Example 99 was prepared according to methods F, I and G utilizing 4-benzyloxymethyl-1-bromobenzene instead of 4-benzyloxy-1-bromobenzene. The final deprotection step afforded the above-identified compound as a side product. MS: m/z 363 (M+1)

### Example 100

Synthesis of (6R)-1-(3-chloro-4-[3-(2-methoxybenzyloxy)-propoxy]-phenyl)-6-(naphthalene-2-yloxymethyl)-piperazin-2-one

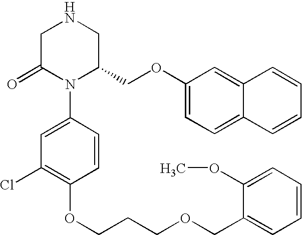

The compound of Example 100 was prepared according to methods F, Q, utilizing 2-chloro-4-iodo-1-[3-(2-methoxybenzyloxy)-propoxy]-benzene instead of 1-benzyloxy-4-iodobenzene, and B. MS: m/z 561 (M+1).

### Example 101

Synthesis of (6R)-1-{4-[3-(2-methoxybenzylsulfanyl)-propoxy]-phenyl}-6-(naphthalene-2-yloxymethyl)-piperazin-2-one

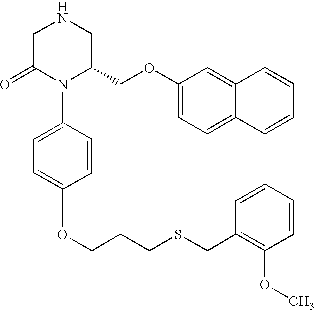

The compound of Example 101 was prepared according to methods A and B utilizing 1-(3-bromopropylsulfanylmethyl)-2-methoxy-benzene instead of 1-(3-iodopropoxymethyl)-2-methoxybenzene. MS: m/z 543 (M+1)

### Example 102

Synthesis of (2R)-1-{4-[3-(2-methoxybenzylsulfanyl)-propoxy]-phenyl}-2-(naphthalene-2-yloxymethyl)-piperazine

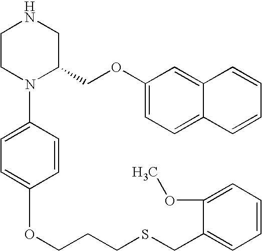

The compound of Example 102 was prepared as in Example 101 followed by reduction of the piperazinone as in method U. MS: m/z 529 (M+1)

### Example 103

Synthesis of (6R)-1-{4-[3-(2-methoxybenzyloxy)-propoxy]-3-methyl-phenyl}-6-(naphthalene-2-yloxymethyl)-piperazin-2-one

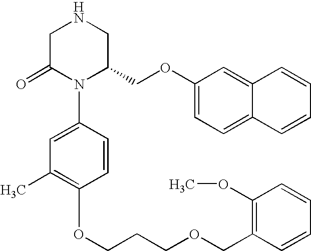

The compound of Example 103 was prepared according to methods F, Q, utilizing 4-iodo-1-[3-(2-methoxybenzyloxy)-propoxy]-2-methylbenzene instead of 1-benzyloxy-4-iodobenzene, and B. MS: m/z 541 (M+1)

### Example 104

Synthesis of (2R)-1-{4-{2-[2-(2-methoxyphenyl)-ethoxy]-ethoxy}-ethoxy}-phenyl)-2-(naphthalene-2-yloxymethyl)-piperazine

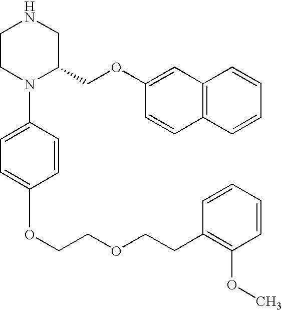

The compound of Example 104 was prepared according to methods F, I, step (v) of G, utilizing 4-[2-(2-methoxyphenethoxy)-ethoxy)-1-bromobenzene instead of 4-benzyloxymethyl-1-bromobenzene, and step (ii) of B. MS: m/z 513 (M+1)

### Example 105

Synthesis of (6R)-1-{4-[3-(2-methoxybenzyloxy)-propoxy]-phenyl}-6-(7-methoxynaphthalen-2-yloxymethyl)-piperazin-2-one

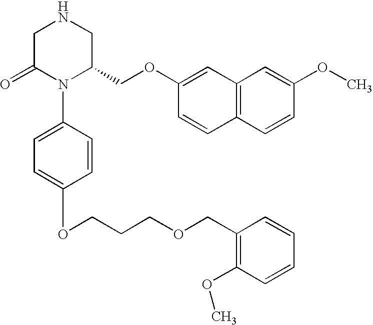

The compound of Example 105 was prepared according to methods A and B utilizing 7-methoxy-2-naphthol instead of 2-naphthol. MS: m/z 557 (M+1)

### Example 106

Synthesis of (6R)-6-(biphenyl-3-yloxymethyl)-1-{4-[3-(2-methoxybenzyloxy)-propoxy]-phenyl}-piperazin-2-one

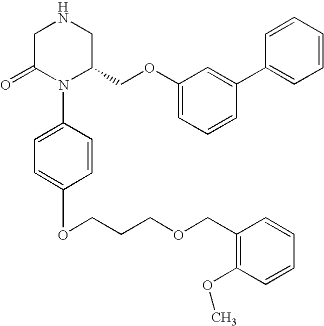

The compound of Example 106 was prepared according to methods A and B utilizing 3-hydroxy biphenyl instead of 2-naphthol. MS: m/z 553 (M+1)

### Example 107

Synthesis of (6R)-1-(3-methoxy-4-[3-(2-methoxybenzyloxy)-propoxy]-phenyl)-6-(naphthalene-2-yloxymethyl)-piperazin-2-one

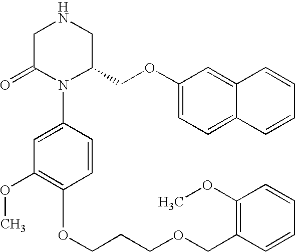

The compound of Example 107 was prepared according to methods F, Q, utilizing 4-bromo-2-methoxy-1-[3-(2-methoxybenzyloxy)-propoxy]-benzene instead of 1-benzyloxy-4-iodobenzene, and B. MS: m/z 557 (M+1)

### Example 108

Synthesis of (2R)-1-{4-[4-(2-methoxyphenoxy)-butoxy]-phenyl}-2-(naphthalene-2-yloxymethyl)-piperazine

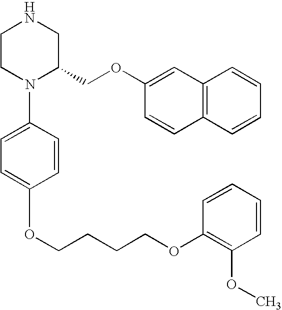

The compound of Example 108 was prepared according to methods F, I, step (v) of G, utilizing 4-[4-(2-methoxyphenoxy)-butoxy)-1-bromobenzene instead of 4-benzyloxymethyl-1-bromobenzene, and step (ii) of B. MS: m/z 513 (M+1)

### Example 109

Synthesis of (6R)-1-{4-[3-(2-Methoxybenzyloxy)-propoxy]-phenyl}-6-[1-(3-methoxypropyl)-1,2,3,4-tetrahydroquinolin-7-yloxymethyl]-piperazin-2-one

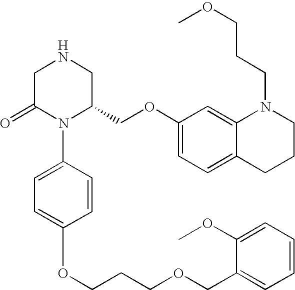

The compound of Example 109 was prepared according to methods A, B and O utilizing 3-bromo-1-methoxypropane instead of 2-bromoethanol. MS: m/z 604.3 (M+1)

### Example 110

Synthesis of {7-[(2R)-1-{4-[3-(2-Methoxybenzyloxy)-propoxy]-phenyl}-6-oxopiperazin-2-ylmethoxy]-3,4-dihydro-2H-quinolin-1-yl}-acetic acid methyl ester

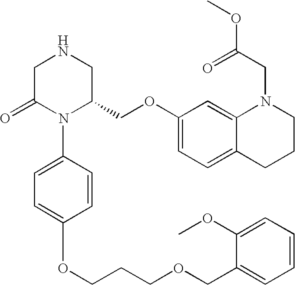

The compound of Example 110 was prepared according to methods A, B and O utilizing methyl 2-bromoacetate instead of 2-bromoethanol. MS: m/z 604.2 (M+1)

### Example 111

Synthesis of 7-([2R]-1-{4-[3-(2-Methoxybenzyloxy)-propoxy]-phenyl}-6-oxopiperazin-2-ylmethoxy)-1-(3-methoxypropyl)-3,4-dihydro-1H-quinolin-2-one

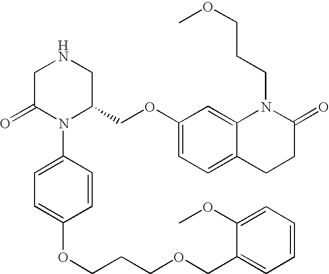

The compound of Example 111 was prepared according to methods A and B utilizing 7-hydroxy-1-(3-methoxypropyl)-3,4-dihydro-1H-quinolin-2-one (prepared as detailed below) instead of 2-naphthol. MS: m/z 618.2 (M+1)

A mixture of 327 mg (2.00 mmole) of 7-hydroxy-3,4-dihydro-1H-quinolin-2-one (prepared as detailed in WO 01/77100 utilizing 3-aminophenol in place of 4-aminophenol) and 415 mg (3.0 mmole) of potassium carbonate in 8 mL of CH₃CN was heated at reflux for 22 hr. After cooling to room temperature, the mixture was diluted with EtOAc, filtered through celite, and concentrated. Purification by flash column chromatography (SiO₂, 20% EtOAc/hexanes gradient to 70% EtOAc/hexanes) gave 380 mg (75%) of 7-benzyloxy-3,4-dihydro-1H-quinolin-2-one as a white solid. MS: m/z 254.1 (M+1)

7-Benzyloxy-3,4-dihydro-1H-quinolin-2-one (379 mg, 1.50 mmole) was dissolved in 8 mL of anhydrous DMF under a N₂ atmosphere and cooled in an ice bath. NaH (57 mg, 2.24 mmole) was added in a single portion, and the resulting gray suspension was stirred at 0° C. for 10 min. 3-Bromo-1-methoxypropane (275 mg, 1.8 mmole) was added, and the reaction mixture was stirred at room temperature for 3 hr. Excess hydride was quenched by the addition of a large excess of H₂O, and the aqueous layer was extracted with EtOAc (3×). The combined organic layers were washed with H₂O (2×) and brine, dried over MgSO₄, filtered, and concentrated. Purification by flash column chromatography (SiO₂, 10% EtOAc/hexanes gradient to 40% EtOAc/hexanes) provided 382 mg (78%) of 7-benzyloxy-1-(3-methoxypropyl)-3,4-dihydro-1H-quinolin-2-one as a clear viscous oil. MS: m/z 326.1 (M+1)

7-Benzyloxy-1-(3-methoxypropyl)-3,4-dihydro-1H-quinolin-2-one (355 mg, 1.09 mmole) was dissolved in 50 mL of MeOH and hydrogenated over 0.1 g of 20% Pd/C at 50 psi for 16 hr. The catalyst was removed by filtration through celite, and the solution was concentrated to dryness to afford 259 mg (100%) of 7-hydroxy-1-(3-methoxypropyl)-3,4-dihydro-1H-quinolin-2-one as a light brown viscous oil which was used without further purification. MS: m/z 236.1 (M+1)

### Example 112

Synthesis of 7-([2S]-1-{4-[3-(2-Methoxybenzyloxy)-propoxy]-phenyl}-6-oxopiperazin-2-ylmethoxy)-1′-(3-methoxypropyl)-3,4-dihydro-1H-quinolin-2-one

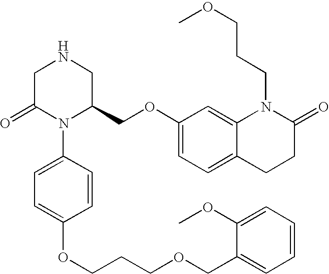

The compound of Example 112 was prepared according to methods A and B utilizing 7-hydroxy-1-(3-methoxypropyl)-3,4-dihydro-1H-quinolin-2-one (prepared as detailed in Example 111) instead of 2-naphthol and intermediates of S configuration. MS: m/z 618.2 (M+1)

### Example 113

Synthesis of [5-([2S]-1-{4-[3-(2-Methoxybenzyloxy)-propoxy]-phenyl}-6-oxopiperazin-2-ylmethoxy)-indol-1-yl]-acetic acid methyl ester

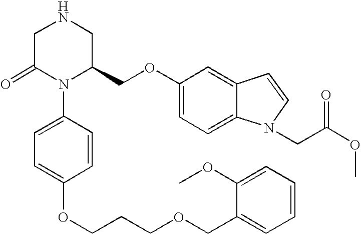

The compound of Example 113 was prepared according to method P utilizing (5-hydroxyindol-1-yl)-acetic acid methyl ester (prepared as detailed in WO 02/060438) instead of 2-(6-hydroxyindol-1-yl)ethyl ester and intermediates of S configuration. MS: m/z 588.2 (M+1)

### Example 114

Synthesis of 7-([2R]-1-{4-[3-(2-Methoxybenzyloxy)-propoxy]-phenyl}-6-oxopiperazin-2-ylmethoxy)-1-(3-methoxypropyl)-1H-[1,8]naphthyridin-2-one

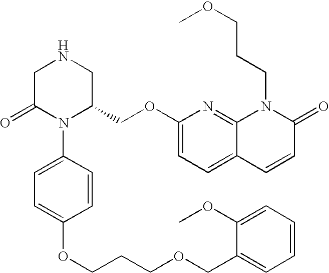

The compound of Example 114 was prepared according to methods A and T utilizing 7-chloro-1-(3-methoxypropyl)-1H-[1,8]naphthyridin-2-one (prepared as detailed below) instead of 2-chloroquinoline. MS: m/z 617.6 (M+1)

A suspension of 300 mg (1.66 mmole) of 7-chloro-1H-[1,8]naphthyridin-2-one (J. Org. Chem. 1990, 55, 4744-4750) in 5 mL of anhydrous DMF was cooled to 0° C. in an ice bath under a N₂ atmosphere. A solution of 1.0 M lithium bis(trimethylsilyl)amide in THF (2.0 mL, 2.0 mmole) was added in a dropwise fashion. After stirring at 0° C. for 5 min., 381 mg (2.49 mmole) of 1-bromo-3-methoxypropane was added. The ice bath was removed, and the reaction mixture was stirred at room temperature for 5 min. An additional 5 mL of anhydrous DMF was added, and the heterogeneous mixture was stirred at room temperature for 18 hr. The reaction mixture was diluted with EtOAc, and washed with H₂O (3×) and brine. The organic layer was dried over MgSO₄, filtered, and concentrated. Purification by flash column chromatography (SiO₂, 40% EtOAc/hexanes gradient to 60% EtOAc/hexanes) gave 304 mg (72%) of 7-chloro-1-(3-methoxypropyl)-1H-[1,8]naphthyridin-2-one. MS: m/z 253.1, 255.1 (M+1)

### Example 115

Synthesis of [5-([2R]-1-{4-[3-(2-Methoxybenzyloxy)-propoxy]-phenyl}-6-oxopiperazin-2-ylmethylsulfanyl)-2-oxobenzooxazol-3-yl]-acetic acid methyl ester

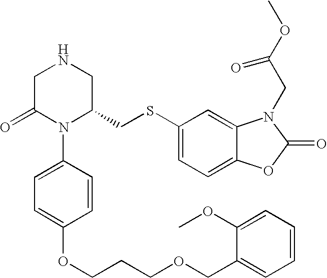

The compound of Example 115 was prepared according to methods A and P utilizing (5-mercapto-2-oxobenzooxazol-3-yl)-acetic acid methyl ester (prepared as detailed below) instead of 2-(6-hydroxyindol-1-yl)ethyl ester. MS: m/z 622.0 (M+1)

A solution of 1.0 g (4.83 mmole) of (2-Oxobenzooxazol-3-yl)-acetic acid methyl ester (J. Praktish. Chemie 1966, 33, 130-138) in 4 mL of chloroform was added to 3.0 mL (4.83 mmole) of chlorosulfonic acid at 0° C. in an ice-bath with stirring. The reaction mixture was stirred at room temperature for 16 hr. The dark reaction mixture was added slowly to a stirring ice-water mixture (100 mL). The aqueous layer was extracted with CH₂Cl₂ (3×). The combined organic layers were washed with water, brine, dried over MgSO₄, and condensed to give 1.44 g (97%) of (5-chlorosulfonyl-2-oxobenzooxazol-3-yl) acetic acid methyl ester as a purple oil, which was used without further purification. MS: m/z 304.1 (M+1)

(5-Chlorosulfonyl-2-oxobenzooxazol-3-yl) acetic acid methyl ester (1.1 g, 3.6 mmole) was dissolved in 20 mL of methanol. HCl (20 mL, 4M in dioxane) and 2.14 g (18 mmole) of tin powder were added sequentially, and the mixture was heated at reflux for 4 hr. After concentration of the solvent, the solid residue was diluted with water, which was extracted with CH₂Cl₂ (3×). The combined organic layers were washed with water (2×), brine, dried over MgSO₄, and condensed to give 840 mg (97%) of (5-mercapto-2-oxobenzooxazol-3-yl)-acetic acid methyl ester as a yellow oil, which was used without further purification. MS: m/z 239.1 (M+1)

### Example 116

Synthesis of [7-([2R]-1-{4-[3-(2-Methoxybenzyloxy)-propoxy]-phenyl}-6-oxopiperazin-2-ylmethoxy)-2-oxo-3,4-dihydro-2H-quinolin-1-yl]-acetic acid methyl ester

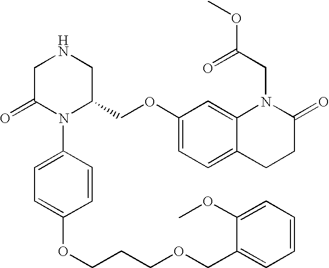

The compound of Example 116 was prepared according to methods A and P utilizing (7-hydroxy-2-oxo-3,4-dihydro-2H-quinolin-1-yl)-acetic acid methyl ester instead of 2-(6-hydroxyindol-1-yl)ethyl ester. MS: m/z 618.1 (M+1)

(7-Hydroxy-2-oxo-3,4-dihydro-2H-quinolin-1-yl)-acetic acid methyl ester was prepared as detailed in Example 111 using methyl 2-bromoacetate instead of 1-bromo-3-methoxypropane. MS: m/z 236.1 (M+1)

### Example 117

Synthesis of N-{2-[7-([2R]-1-{4-[3-(2-Methoxybenzyloxy)-propoxy]-phenyl}-6-oxopiperazin-2-ylmethoxy)-2-oxo-3,4-dihydro-2H-quinolin-1-yl]-ethyl}-acetamide

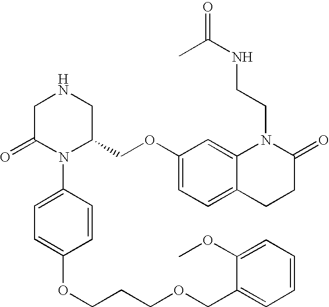

The compound of Example 117 was prepared according to methods A and P utilizing N-[2-(7-hydroxy-2-oxo-3,4-dihydro-2H-quinolin-1-yl)-ethyl]-acetamide (prepared as detailed below) instead of 2-(6-hydroxyindol-1-yl)ethyl ester. MS: m/z 631.1 (M+1)

(7-Benzyloxy-2-oxo-3,4-dihydro-2H-quinolin-1-yl)-acetonitrile (1.9 g, 6.5 mmole, prepared as detailed in Example 111 using 2-bromoacetonitrile instead of 1-bromo-3-methoxypropane) was hydrogenated in EtOH/Ac₂O over 1.5 g of Raney nickel at 100 psi of H₂ pressure for 16 hr. The catalyst was removed by filtration through celite, and the solution was concentrated. Purification by flash column chromatography (Al₂O₃, 2:25:73 MeOH/EtOAc/hexanes) gave 2.0 g (91%) of N-[2-(7-benzyloxy-2-oxo-3,4-dihydro-2H-quinolin-1-yl)-ethyl]-acetamide as an off-white solid. MS: m/z 339.1 (M+1)

N-[2-(7-Hydroxy-2-oxo-3,4-dihydro-2H-quinolin-1-yl)-ethyl]-acetamide was prepared as detailed in Example 111 using N-[2-(7-benzyloxy-2-oxo-3,4-dihydro-2H-quinolin-1-yl)-ethyl]-acetamide instead of 7-benzyloxy-1-(3-methoxypropyl)-3,4-dihydro-1H-quinolin-2-one. MS: m/z 249.1 (M+1)

### Example 118

Synthesis of [6-([2R]-1-{4-[3-(2-Methoxybenzyloxy)-propoxy]-phenyl}-6-oxo-piperazin-2-ylmethoxy)-3-oxo-2,3-dihydro-benzo[1,4]oxazin-4-yl]-acetic acid methyl ester

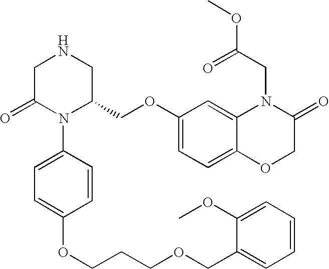

The compound of Example 118 was prepared according to methods A and P utilizing (6-hydroxy-3-oxo-2,3-dihydrobenzo[1,4]oxazin-4-yl)-acetic acid methyl ester (prepared as detailed below) instead of 2-(6-hydroxyindol-1-yl)ethyl ester. MS: m/z 620.1 (M+1)

A mixture of 2 g (10 mmole) of 6-acetyl-2H-1,4-benzoxazin-3(4H)-one, 6 g (34 mmole) of m-CPBA, and 6 g of sodium bicarbonate was stirred at room temperature for 16 hr. DCM (100 mL), an additional 4 g of m-CPBA (4 g) and 2.4 g of sodium bicarbonate was added, and the suspension was stirred for 24 hr. The solids were removed by filtration through celite, and the filtrate was concentrated. Purification by flash column chromatography (SiO₂, 5% EtOAc/hexanes gradient to 45% EtOAc/hexanes) gave 1.75 g (84%) of acetic acid 3-oxo-3,4-dihydro-2H-benzo[1,4]oxazin-6-yl ester as a white solid. MS: m/z 208.1 (M+1)

A solution of 2.35 g (11.3 mmole) of acetic acid 3-oxo-3,4-dihydro-2H-benzo[1,4]oxazin-6-yl ester was dissolved in 25 mL of MeOH and 25 mL of THF. An 1N aqueous solution NaOH (15 mL) was added, and the reaction mixture was heated at reflux for 1 h. The solvent was removed by concentration in vacuo, and the residue was redissolved in 150 mL of water, which was acidified with 1N HCl to pH 2. The resulting precipitate was collected on a fritted funnel and dried under vacuum to give 1.5 g (80%) of 6-hydroxy-4H-benzo[1,4]oxazin-3-one as a white solid. MS: m/z 166.1 (M+1)

(6-Hydroxy-3-oxo-2,3-dihydrobenzo[1,4]oxazin-4-yl)-acetic acid methyl ester was prepared as detailed in Example 111 using methyl 2-bromoacetate instead of 1-bromo-3-methoxypropane. MS: m/z 238.1 (M+1)

### Example 119

Synthesis of 3-[5-([2R]-1-{4-[3-(2-Methoxybenzyloxy)-propoxy]-phenyl}-6-oxopiperazin-2-ylmethoxy)-indol-1-yl]-propionic acid methyl ester

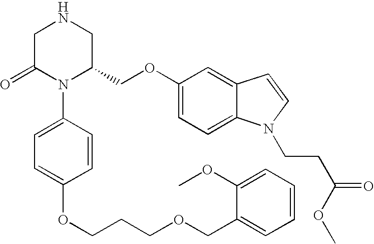

The compound of Example 119 was prepared according to methods A and P utilizing 3-(5-hydroxy-indol-1-yl)-propionic acid methyl ester instead of 2-(6-hydroxyindol-1-yl)ethyl ester. MS: m/z 602.1 (M+1) 3-(5-Hydroxy-indol-1-yl)-propionic acid methyl ester was prepared according to method P using 5-benzyloxyindole instead of 6-benzyloxyindole and ethyl 3-bromopropionate in place of 2-bromoethyl acetate. MS: m/z 220.1 (M+1)

### Example 120

Synthesis of N-{2-[5-([2R]-1-{4-[3-(2-Methoxybenzyloxy)-propoxy]-phenyl}-6-oxopiperazin-2-ylmethoxy)-indol-1-yl]-ethyl}-acetamide

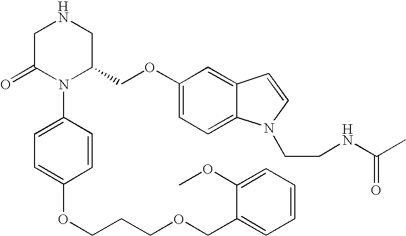

The compound of Example 120 was prepared according to methods A and P utilizing N-[2-(5-hydroxyindol-1-yl)-ethyl]-acetamide instead of 2-(6-hydroxyindol-1-yl)ethyl ester. MS: m/z 600.1 (M+1).

N-[2-(5-Hydroxyindol-1-yl)-ethyl]-acetamide was prepared as detailed in Example 117 using 5-benzyloxyindole in place of 7-benzyloxy-3,4-dihydro-1H-quinolin-2-one. MS: m/z 219.1 (M+1).

### Example 121

Synthesis of [5-([2R]-1-{4-[3-(2-Methoxybenzyloxy)-propoxy]-phenyl}-6-oxopiperazin-2-ylmethoxy)-2-methyl-indol-1-yl]-acetic acid methyl ester

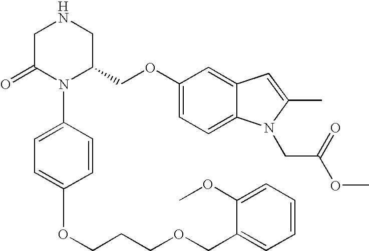

The compound of Example 121 was prepared according to methods A and P utilizing (5-hydroxy-2-methylindol-1-yl)-acetic acid methyl ester (prepared as described below) instead of 2-(6-hydroxyindol-1-yl)ethyl ester. MS: m/z 602.1 (M+1)

A solution of 1.5 g (6.4 mmole) of (5-methoxy-2-methylindol-1-yl)-acetic acid methyl ester (prepared as detailed in method P utilizing 5-methoxy-2-methyl indole in place of 6-benzyloxyindole and methyl 2-bromoacetate instead of 2-bromoethyl acetate) in 35 mL of anhydrous CH₂Cl₂ was cooled in an ice bath and treated with 8 mL of 1 M BBr₃ in CH₂Cl₂. The reaction mixture was stirred at room temperature for 1 hr. Excess acid was quenched by the addition of a concentrated aqueous NaHCO₃ solution. The organic layer was washed with water and brine, dried over MgSO₄, filtered, and concentrated. Purification by flash column chromatography (SiO₂, 15% EtOAc/hexanes gradient to 85% EtOAc/hexanes) afforded 0.55 g (39%) of (5-hydroxy-2-methylindol-1-yl)-acetic acid methyl ester as a dark colored solid. MS: m/z 220.1 (M+1)

### Example 122

Synthesis of 3-[7-([2R]-1-{4-[3-(2-Methoxybenzyloxy)-propoxy]-phenyl}-6-oxopiperazin-2-ylmethoxy)-3,4-dihydro-2H-quinolin-1-yl]-propionic acid methyl ester

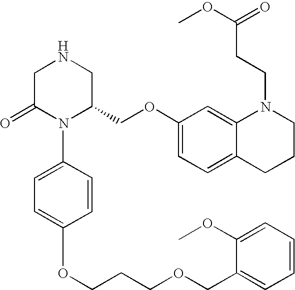

The compound of Example 122 was prepared according to methods A and P utilizing 3-(7-hydroxy-3,4-dihydro-2H-quinolin-1-yl)-propionic acid methyl ester (prepared as described below) instead of 2-(6-hydroxyindol-1-yl)ethyl ester. MS: m/z 605 (M+1)

A solution of 1.50 g (4.62 mmole) of 3-(7-Benzyloxy-3,4-dihydro-2H-quinolin-1-yl)-propionic acid methyl ester (prepared from 7-benzyloxyquinoline (Chem. Research. Tox. 2002, 15, 806-814) as described in steps (i) and (ii) of Method O) in methanol was hydrogenated over 10% Pd/C at 55 psi of hydrogen pressure for 20 hr. The catalyst was removed by filtration through celite, and the volatiles were removed by concentration in vacuo to give 0.90 g (82%) of 3-(7-hydroxy-3,4-dihydro-2H-quinolin-1-yl)-propionic acid methyl ester as a viscous oil. ¹H NMR (400 MHz, CDCl₃): δ 7.26 (br s, 1H), 6.78 (d, 1H), 6.10-6.07 (m, 2H), 3.69 (s, 3H), 3.56 (m, 2H), 2.25 (br s, 2H), 2.65-2.58 (m, 4H), 1.90 (br s, 2H)

### Example 123

Synthesis of Propionic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester

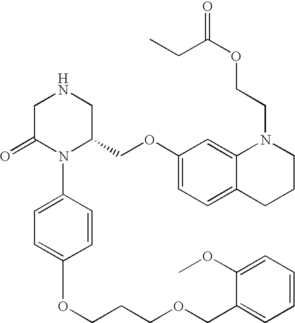

The compound of Example 123 was prepared according to methods A and P utilizing propionic acid 2-(7-hydroxy-3,4-dihydro-2H-quinolin-1-yl)-ethyl ester (prepared as described below) instead of 2-(6-hydroxyindol-1-yl)ethyl ester. ¹H NMR (400 MHz, CDCl₃) δ: 7.36-7.34 (m, 1H), 7.26-7.23 (m, 1H), 7.15-7.11 (m, 2H), 6.94-6.89 (m, 3H), 6.86-6.84 (m, 1H), 6.78 (d, 1H, J=7.8 Hz), 6.15 (d, 1H, J=2.4 Hz), 6.01-5.98 (m, 1H), 4.56 (s, 2H), 4.23 (t, 2H, J=6.3 Hz), 4.12-4.06 (m, 3H), 3.94-3.85 (m, 2H), 3.79 (s, 3H), 3.74-3.62 (m, 4H), 3.45-3.44 (m, 3H), 3.36-3.29 (m, 3H, J=5.9 Hz), 2.66 (t, 2H, J=6.3 Hz), 2.30 (q, 2H, J=7.8 Hz), 2.12-2.06 (m, 2H,), 1.92-1.86 (m, 3H), 1.12 (t, 3H, J=7.8 Hz)

A solution of 452 mg (2.2 mmole) of 2-(7-methoxy-3,4-dihydro-2H-quinolin-1-yl)-ethanol (prepared by 7-methoxyquinoline using steps (i) and (ii) of Method O) in 50 mL of anhydrous CH₂Cl₂ was cooled in a dry ice/acetone bath under a N₂ atmosphere. BBr₃ (4.46 g, 17.8 mmol) was added dropwise. The cold bath was removed, and the reaction mixture was stirred at room temperature for 5 h. The mixture was cooled in an ice bath, and excess BBr₃ was quenched by the dropwise addition of 20 mL of CH₃OH. The resulting solution was stirred at room temperature for 2 h. After removal of the solvents, the residue was treated with H₂O and ethyl acetate. The combined organic layers were washed with H₂O, dried over Na₂SO₄, and concentrated give 0.34 g (83%) of 1-(2-hydroxyeethyl)-1,2,3,4-tetrahydro-quinolin-7-ol, which was used without further purification. ¹H NMR (400 MHz, CDCl₃) δ: 6.79 (d, 1H, J=7.8 Hz), 6.19 (d, 1H, J=2.4 Hz), 6.11-6.09 (m, 1H), 3.80 (t, 2H, J=5.9 Hz), 3.39 (t, 2H, J=5.8 Hz), 3.28 (t, 2H, J=5.4 Hz), 2.68 (t, 2H, J=6.3 Hz), 1.93-1.90 (m, 2H)

A mixture of 0.35 g (1.8 mmole) of 1-(2-hydroxyeethyl)-1,2,3,4-tetrahydro-quinolin-7-ol, 0.52 g (4.0 mmole) of (CH₃CH₂CO)₂O, 22 mg (0.18 mmole) of DMAP, and 3 mL of pyridine in 10 mL of CH₂Cl₂ was stirred at room temperature for 4 h. The mixture was diluted with dichloromethane and H₂O. The organic layer was washed with H₂O, dried over Na₂SO₄, and concentrated under vacuum to give 0.55 g (100%) propionic acid 1-(2-propionyloxyethyl)-1,2,3,4-tetrahydro-quinolin-7-yl ester, which was used without further purification. ¹H NMR (400 MHz, CDCl₃) δ: 6.91-6.89 (m, 1H), 6.29-6.27 (m, 2H), 4.25 (t, 2H, J=6.3 Hz), 3.49 (t, 2H, J=6.3 Hz), 3.33 (t, 2H, J=5.8 Hz), 2.72 (t, 2H, J=6.3 Hz), 2.70 (q, 2H, J=7.3 Hz), 2.50 (q, 2H, J=7.3 Hz), 1.94-1.91 (m, 2H), 1.26 (t, 3H, J=7.3 Hz), 1.14 (t, 3H, J=7.3 Hz)

A mixture of 0.55 g (1.8 mmole) of propionic acid 1-(2-propionyloxyethyl)-1,2,3,4-tetrahydro-quinolin-7-yl ester and NaHCO₃ (0.19 g, 1.8 mmol) in a mixture of H₂O (5 mL) and CH₃OH (10 mL) was stirred at room temperature for 16 hr, diluted with CH₂Cl₂ and H₂O. The organic layer was washed with H₂O, dried over Na₂SO₄, and concentrated under vacuum. Purification by flash column chromatography (SiO₂, 10% EtOAc/hexanes) gave 0.17 g (38%) of propionic acid 2-(7-hydroxy-3,4-dihydro-2H-quinolin-1-yl)-ethyl ester. ¹H NMR (400 MHz, CDCl₃) δ: 6.78 (d, 1H, J=7.8 Hz), 6.16 (d, 1H, J=2.4 Hz), 6.09-6.06 (m, 1H), 5.10 (s, 1H), 4.25 (t, 2H, J=6.3 Hz), 3.48 (t, 2H, J=6.3 Hz), 3.31 (t, 2H, J=5.8 Hz), 2.67 (t, 2H, J=6.3 Hz), 2.34 (q, 2H, J=7.3 Hz), 1.92-1.89 (m, 2H), 1.11 (t, 3H, J=7.3 Hz)

### Example 124

Synthesis of N-{2-[7-([2R]-1-{4-[3-(2-Methoxybenzyloxy)-propoxy]-phenyl}-6-oxopiperazin-2-ylmethoxy)-3,4-dihydro-2H-quinolin-1-yl]-ethyl}-acetamide

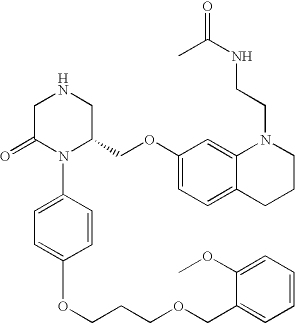

The compound of Example 124 was prepared according to methods A and P utilizing N-[2-(7-hydroxy-3,4-dihydro-2H-quinolin-1-yl)-ethyl]-acetamide (prepared as described below) instead of 2-(6-hydroxyindol-1-yl)ethyl ester. MS: m/z 617.29 (M+1)

A solution of 3.8 g (9.2 mmole) of 2-[2-(7-benzyloxy-3,4-dihydro-2H-quinolin-1-yl)-ethyl]-isoindole-1,3-dione (prepared from 7-benzyloxyquinoline and N-(2-bromoethyl)phthalimide as described in Example 122) in 100 mL of ethanol was treated with 1.4 mL (27 mmole) hydrazine hydrate. The reaction mixture was heated at 88° C. for 6 hr. After cooling to room temperature, The white precipitate was dissolved by addition of 25 mL of 6 N hydrochloric acid. The resulting light blue solution was neutralized with aqueous NaOH, and extracted with EtOac (2×). The combined organic extracts were dried (Na₂SO₄) and the solvents were evaporated to give 2.5 g (100%) of 2-(7-benzyloxy-3,4-dihydro-2H-quinolin-1-yl)-ethylamine, which was used without further purification. ¹H NMR (400 MHz, CDCl₃): δ 7.49-7.24 (m, 5H), 6.83 (d, 1H), 6.28-6.16 (m, 2H), 5.03 (s, 2H), 3.38-3.18 (m, 4H), 2.91 (t, 2H), 2.66 (t, 2H), 1.99-1.82 (m, 2H), 1.51-1.32 (br s, 2H)

A solution of 2.25 g (7.98 mmole) of 2-(7-benzyloxy-3,4-dihydro-2H-quinolin-1-yl)-ethylamine and 1.77 mL (12.8 mmole) of Et₃N in 30 mL of anhydrous CH₂Cl₂ was cooled in an ice bath. Acetyl chloride (0.8 mL, 11.2 mmol) was then added dropwise, and the resulting mixture was stirred at 0° C. for 3 hr. Excess acid was quenched by the addition of 20 mL of saturated aqueous NaHCO₃. The aqueous layer was extracted with CH₂Cl₂ (2×). The combined organic layers were dried over Na₂SO₄, filtered, and concentrated to afford 2.08 g (83%) of N-[2-(7-benzyloxy-3,4-dihydro-2H-quinolin-1-yl)-ethyl]-acetamide as a light yellow oil. ¹H NMR (400 MHz, CDCl₃): δ 7.53-7.22 (m, 5H), 6.84 (d, 1H), 6.32-6.17 (m, 2H), 5.84-5.68 (br s, 1H), 5.03 (s, 2H), 3.45-3.18 (m, 6H), 2.66 (t, 2H), 1.98-1.82 (m, including a singlet at δ 1.88, 5H in total)

N-[2-(7-hydroxy-3,4-dihydro-2H-quinolin-1-yl)-ethyl]-acetamide was prepared from N-[2-(7-benzyloxy-3,4-dihydro-2H-quinolin-1-yl)-ethyl]-acetamide as described in step (ii) of Method P. ¹H NMR (400 MHz, CDCl₃): δ 6.79 (d, 1H), 6.27 (s, 1H), 6.17-6.11 (m, 1H), 6.08-5.92 (br s, 1H), 3.53-3.18 (m, 6H), 2.65 (t, 2H), 2.02-1.81 (m, including a singlet at δ 1.98, 6H in total)

### Example 125

Synthesis of N-{2-[7-([2R]-1-{4-[3-(2-Methoxybenzyloxy)-propoxy]-phenyl}-piperazin-2-ylmethoxy)-3,4-dihydro-2H-quinolin-1-yl]-ethyl}-acetamide

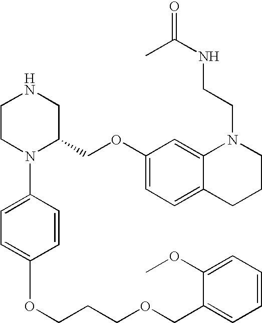

The compound of Example 125 was prepared from (3R)-3-[1-(2-acetylaminoethyl)-1,2,3,4-tetrahydroquinolin-7-yloxymethyl]-4-{4-[3-(2-methoxybenzyloxy)-propoxy]-phenyl}-piperazine-1-carboxylic acid tert-butyl ester as described below. MS: m/z 603.31 (M+1)

A solution of 3.8 g (6.0 mmole) of (3R)-4-{4-[3-(2-methoxybenzyloxy)-propoxy]-phenyl}-3-(1,2,3,4-tetrahydro-quinolin-7-yloxymethyl)-piperazine-1-carboxylic acid tert-butyl ester (prepared as described in Method A, step (iii) of Method P, and step (i) of Method O) in anhydrous THF was treated with 15 mL (30 mmole) of 2.0 M BH₃.DMS in THF dropwise at room temperature under N₂. The resulting mixture was stirred at 50-60° C. for 6 hr. The cooled mixture was treated with 300 mL of 10% potassium sodium tartrate. The resulting biphasic mixture was stirred at 50-60° C. for 16 hr and extracted with EtOAc. The organic layer was washed with H₂O, brine, dried over Na₂SO₄ and concentrated. Purification by flash column chromatography (SiO₂, 30% EtOAc/hexanes gradient to 50% EtOAc/hexanes) gave 2.0 g (54%) of (3R)-4-{4-[3-(2-methoxybenzyloxy)-propoxy]-phenyl}-3-(1,2,3,4-tetrahydro-quinolin-7-yloxymethyl)-piperazine-1-carboxylic acid tert-butyl ester. MS: m/z: 618.36 (M+1)

(3R)-3-(1-Cyanomethyl-1,2,3,4-tetrahydroquinolin-7-yloxymethyl)-4-{4-[3-(2-methoxybenzyloxy)-propoxy]-phenyl}-piperazine-1-carboxylic acid tert-butyl ester was prepared from (3R)-4-{4-[3-(2-methoxybenzyloxy)-propoxy]-phenyl}-3-(1,2,3,4-tetrahydro-quinolin-7-yloxymethyl)-piperazine-1-carboxylic acid tert-butyl ester as described in step (ii) of Method O

A mixture of 0.7 g (1.3 mmole) of (3R)-3-(1-cyanomethyl-1,2,3,4-tetrahydroquinolin-7-yloxymethyl)-4-{4-[3-(2-methoxybenzyloxy)-propoxy]-phenyl}-piperazine-1-carboxylic acid tert-butyl ester, 1 mL of Raney-Nickel, 10 mL of THF, and 5 mL of AcOH was shaken under 50 psi of H₂ pressure for 16 hr. The catalyst was remove by filtration through a pad of celite, and the filtrate was concentrated under vacuum. Purification by flash column chromatography (SiO₂, 50% EtOAc/hexanes then 5% CH₃OH/CH₂Cl₂) afforded 0.35 g (87%) of (3R)-3-[1-(2-acetylaminoethyl)-1,2,3,4-tetrahydroquinolin-7-yloxymethyl]-4-{4-[3-(2-methoxybenzyloxy)-propoxy]-phenyl}-piperazine-1-carboxylic acid tert-butyl ester. MS: m/z: 703.38 (M+1)

### Example 126

Synthesis of N-{2-[7-([2R]-1-{4-[3-(2-Fluorobenzyloxy)-propoxy]-phenyl}-6-oxopiperazin-2-ylmethoxy)-3,4-dihydro-2H-quinolin-1-yl]-ethyl}-acetamide

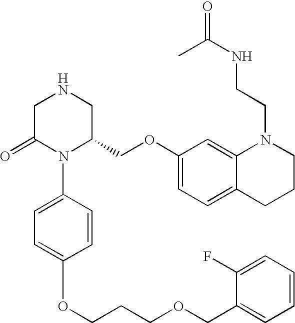

The compound of Example 126 was prepared according to methods A, E and P utilizing 2-fluorobenzaldehyde instead of 2-methoxybenzaldehyde and N-[2-(7-hydroxy-3,4-dihydro-2H-quinolin-1-yl)-ethyl]-acetamide (prepared as described in Example 124) instead of 2-(6-hydroxyindol-1-yl)ethyl ester. MS: m/z 605.26 (M+1)

### Example 127

Synthesis of 7-([2R]-1-{4-[3-(2-Methoxybenzyloxy)-propoxy]-phenyl}-6-oxo-piperazin-2-ylmethoxy)-1-(3-methoxypropyl)-3,4-dihydro-1H-quinazolin-2-one

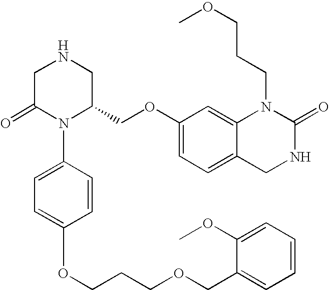

The compound of Example 127 was prepared according to methods A and P utilizing 7-benzyloxy-1-(3-methoxypropyl)-3,4-dihydro-1H-quinazolin-2-one (prepared as described below) instead of 2-(6-benzyloxyindol-1-yl)ethyl ester. MS: m/z 619 (M+1)

A mixture of 5.00 g (22 mmole) of 4-benzyloxy-2-fluorobenzonitrile, 7.88 g (88.1 mmole) of 3-methoxy-1-propylamine, and 11.39 g (88.1 mmole) of diisopropylethylamine was heated at 120° C. under microwave radiation for 3 h. Volatiles were removed under reduced pressure. The residue was purified by flash column chromatography (SiO₂, 25% EtOAc/hexanes) to give 6.20 g (68%) of 4-benzyloxy-2-(3-methoxypropylamino)-benzonitrile as a white solid. MS: m/z 297.1 (M+1)

To a stirred suspension of 4.49 g (118 mmole) of lithium aluminum hydride in 40 mL of anhydrous THF was added a solution of 3.50 g (11.82 mmole) of 4-benzyloxy-2-(3-methoxypropylamino)-benzonitrile in 20 mL of anhydrous THF at room temperature. The reaction mixture was stirred at room temperature for 24 h and then quenched with 4.5 mL of water, 4.5 mL of 15% NaOH solution and 15 mL of water. The resulting slurry was filtered through a celite pad, and the residue was washed with CH₂Cl₂ (100 mL). The organic layer was washed with water, dried over Na₂SO₄, filtered, and concentration to give 2.00 g (56%) of (2-aminomethyl-5-benzyloxyphenyl)-(3-methoxypropyl)-amine, which was used without further purification. ¹H NMR (400 MHz, CDCl₃) δ: 7.45-7.26 (m, 5H), 6.92 (d, 1H), 6.31 (d, 1H), 6.24-6.22 (m, 1H), 5.82 (br s, 1H), 5.04 (s, 2H), 3.82 (s, 2H), 3.52 (t, 2H), 3.36 (s, 3H), 3.21 (br s, 2H), 1.95-1.89 (m, 2H), 1.31 (br s, 2H)

A mixture 1.00 g (3.33 mmole) of (2-aminomethyl-5-benzyloxyphenyl)-(3-methoxypropyl)-amine, 0.59 g (3.66 mmole) of carbonyl diimidazole in 30 mL of anhydrous THF was stirred at room temperature for 12 hr. The reaction mixture was diluted with EtOAc, and washed with 1N HCl solution (2×). The organic layer was dried over Na₂SO₄, filtered, and concentrated to provide 1.00 g (92%) of 7-benzyloxy-1-(3-methoxypropyl)-3,4-dihydro-1H-quinazolin-2-one, which was used without further purification. MS: m/z 327.1 (M+1)

### Example 128

Synthesis of 6-([2R]-1-{4-[3-(2-Methoxybenzyloxy)-propoxy]-phenyl}-6-oxo-piperazin-2-ylmethylsulfanyl)-4-(3-methoxypropyl)-4H-benzo[1,4]oxazin-3-one

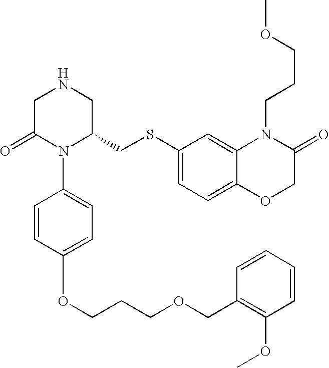

The compound of Example 128 was prepared according to methods A and P utilizing 6-mercapto-4-(3-methoxypropyl)-4H-benzo[1,4]oxazin-3-one (prepared from 4H-benzo[1,4]oxazin-3-one as described in Example 115) instead of 2-(6-benzyloxyindol-1-yl)ethyl ester. MS: m/z: 636 (M+1)

### Example 129

Synthesis of [6-([2R]-1-{4-[3-(2-Methoxybenzyloxy)-propoxy]-phenyl}-6-oxo-piperazin-2-ylmethylsulfanyl)-3-oxo-2,3-dihydro-benzo[1,4]oxazin-4-yl]-acetic acid ethyl ester.

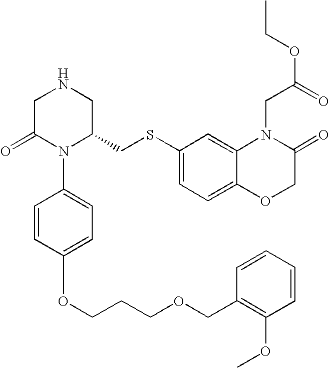

The compound of Example 129 was prepared according to methods A and P utilizing (6-mercapto-3-oxo-2,3-dihydrobenzo[1,4]oxazin-4-yl)-acetic acid ethyl ester (prepared from 4H-benzo[1,4]oxazin-3-one as described in Example 115) instead of 2-(6-benzyloxyindol-1-yl)ethyl ester. MS: m/z: 650 (M+1)

### Example 130

Synthesis of [5-([2R]-1-{4-[3-(2-Methoxybenzyloxy)-propoxy]-phenyl}-6-oxo-piperazin-2-ylmethylsulfanyl)-indan-2-yl]-acetic acid methyl ester.

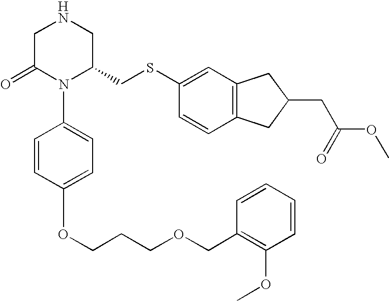

The compound of Example 130 was prepared according to methods A and P utilizing (5-mercapto-indan-2-yl)-acetic acid methyl ester (prepared from (indan-2-yl-acetic acid methyl ester (J. Med. Chem. 2001, 44, 4677-4687) as described in Example 115) instead of 2-(6-benzyloxyindol-1-yl)ethyl ester. MS: m/z: 605 (M+1)

### Example 131

Synthesis of [6-([2R]-1-{4-[3-(2-Methoxybenzyloxy)-propoxy]-phenyl}-6-oxo-piperazin-2-ylmethoxy)-4-methyl-benzo[b]thiophen-3-yl]-acetic acid methyl ester.

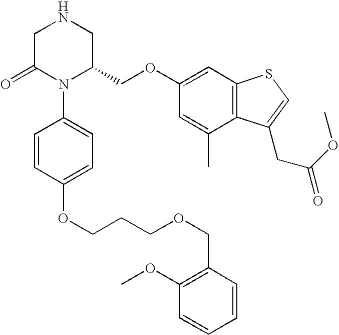

The compound of Example 131 was prepared according to methods A and P utilizing (6-hydroxy-4-methyl-benzo[b]thiophen-3-yl)-acetic acid ethyl ester instead of 2-(6-benzyloxyindol-1-yl)ethyl ester. Tranesterification to the methyl ester occurred during step (iv) of Method P. MS: m/z: 619 (M+1)

### Example 132

Synthesis of [6-([2R]-1-{4-[3-(2-Methoxybenzyloxy)-propoxy]-phenyl}-6-oxo-piperazin-2-ylmethoxy)-indol-1-yl]-acetic acid ethyl ester.

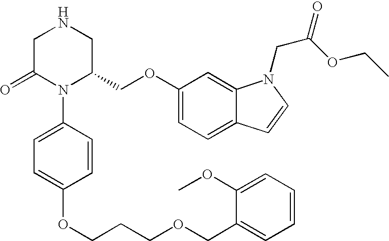

The compound of Example 132 was prepared according to methods A and P utilizing 6-hydroxyindole instead of 2-(6-benzyloxyindol-1-yl)ethyl ester. Alkylation with ethyl 2-bromoacetate was accomplished as described in step (i) of Method P. MS: m/z: 602.28 (M+1)

### Example 133

Synthesis of 3-[6-([2R]-1-{4-[3-(2-Methoxybenzyloxy)-propoxy]-phenyl}-6-oxo-piperazin-2-ylmethoxy)-2,3-dihydro-indol-1-yl]-propionic acid methyl ester.

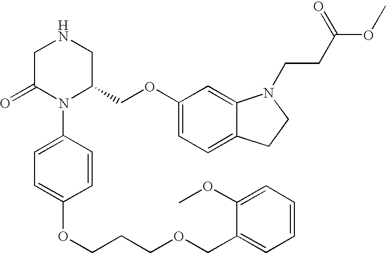

The compound of Example 133 was prepared according to methods A and P utilizing 3-(6-hydroxy-2,3-dihydro-indol-1-yl)-propionic acid methyl ester instead of 2-(6-benzyloxyindol-1-yl)ethyl ester. MS: m/z: 604 (M+1)

What is claimed is:

1. A compound of Formula I 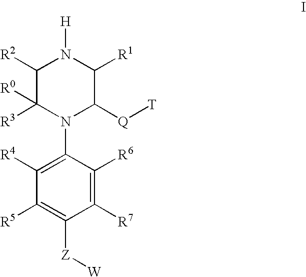
or a pharmaceutically acceptable salt thereof, wherein
R¹ and R² are independently hydrogen or unsubstituted C₁-C₃ alkyl;
R³ is hydrogen, oxo, or thioxo;
R⁰ is hydrogen or unsubstituted C₁-C₃ alkyl provided that when R³ is oxo or thioxo R⁰ is absent;
R⁴, R⁵, R⁶, and R⁷ are independently hydrogen, halogen, carboxyl, substituted or unsubstituted C₁-C₃ alkoxy, or substituted or unsubstituted C₁-C₃ alkyl;
Q is —(CH₂)₁₋₆—C(O)—O—(CH₂)₀₋₆—, —(CH₂)₁₋₆—O—C(O)—(CH₂)₀₋₆—, —(CH₂)₁₋₆—C(O)—NR⁸—(CH₂)₀₋₆—, —(CH₂)₁₋₆—NR⁹—C(O)—(CH₂)₀₋₆-, —(CH₂)₁₋₆—NR¹⁰—S(O)₂—(CH₂)₀₋₆—, —(CH₂)₁₋₆—S(O)₂—NR¹⁰ —(CH₂)₀₋₆—, —(CH₂)₁₋₆—NR¹¹—C(O)—NR¹²—(CH₂)₀₋₆—, or —CH₂—(C₁-C₆ alkylene) wherein 1 to 3 nonadjacent methylene units of the alkylene group are replaced with O, NR¹³, S or a combination thereof;
T is substituted or unsubstituted aryl, substituted or unsubstituted heteroaryl, or substituted or unsubstituted C₁-C₁₂ alkyl;
W is absent, substituted or unsubstituted aryl, or substituted or unsubstituted heteroaryl;
Z is —(CH₂)₀₋₆-cycloalkylene-(CH₂)₀₋₆— wherein 0 to 6 nonadjacent methylene units are replaced with O, NR¹⁶, S or a combination thereof,
—(CH₂)₀₋₆-heterocycloalkylene-(CH₂)₀₋₆— wherein 0 to 6 nonadjacent methylene units are replaced with O, NR¹⁶, S or a combination thereof,
—(CH₂)₀₋₆-arylene-(CH₂)₀₋₆— wherein 0 to 6 nonadjacent methylene units are replaced with O, NR¹⁶, S or a combination thereof,
—(CH₂)₀₋₆-heteroarylene-(CH₂)₀₋₆— wherein 0 to 6 nonadjacent methylene units are replaced with O, NR¹⁶, S or a combination thereof,
—(CH₂)₀₋₆—C(O)—NR¹⁶—(CH₂)₀₋₆- wherein 0 to 6 nonadjacent methylene units are replaced with O, NR¹⁶, S or a combination thereof,
—(CH₂)₀₋₆—NR¹⁶—C(O)—(CH₂)₀₋₆- wherein 0 to 6 nonadjacent methylene units are replaced with O, NR¹⁶, S or a combination thereof, 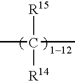
wherein 1 to 6 nonadjacent 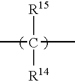
units are replaced with O,
NR¹⁶, S or a combination thereof, or
Z, when W is absent, is hydroxyl, substituted or unsubstituted C₁-C₁₂ alkyl wherein 1 to 6 nonadjacent methylene units are replaced with O,
NR¹⁶, S or a combination thereof, or —(CH₂)₀₋₆—C(O)—NR¹⁶—(CH₂)₀₋₅—CH₃ wherein 0 to 6 nonadjacent methylene units are replaced with O, NR¹⁶, S or a combination thereof;
R⁸, R⁹, R¹⁰, R¹¹, and R¹² are independently hydrogen, substituted or unsubstituted C₁-C₃ alkoxy, or substituted or unsubstituted C₁-C₃ alkyl;
R¹³ and R¹⁶ are independently substituted or unsubstituted C₁-C₃ alkyl or hydrogen; and
R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl wherein 1 to 6 nonadjacent methylene units are replaced with O, or R¹⁴ and R¹⁵ together with the carbon to which they are attached form a 3- to 6-membered cycloalkylene or heterocycloalkylene ring.

2. A compound of claim 1, wherein R¹ and R², are hydrogen and R³ is oxo.

3. A compound of claim 1, wherein R⁴, R⁵, R⁶, and R⁷ are independently hydrogen, halogen, carboxyl, C₁-C₃ alkoxy, or C₁-C₃ alkyl.

4. A compound of claim 3, wherein R⁴, R⁵, R⁶, and R⁷ are independently hydrogen, chlorine, fluorine, carboxyl, methoxy or methyl.

5. A compound of claim 1, wherein R⁴, R⁶, and R⁷ are hydrogen and R⁵ is chlorine, fluorine, carboxyl, methoxy or methyl.

6. A compound of claim 1, wherein Q is —(CH₂)₁₋₆—O—C(O)—(CH₂)₀₋₆—, or —CH₂—(C₁-C₆ alkylene) wherein 1 to 3 nonadjacent methylene units of the alkylene group are replaced with O, NR¹³, S or a combination thereof.

7. A compound of claim 6, wherein Q is —CH₂—(C₁-C₆ alkylene) wherein 1 to 3 nonadjacent methylene units of the alkylene group are replaced with O or S.

8. A compound of claim 7, wherein Q is —CH₂—O—, —CH₂—O—CH₂—CH₂—, —CH₂—O—CH₂—CH₂—CH₂—, —CH₂—S— or —CH₂—O—C(O)—(CH₂)₀₋₆—.

9. A compound of claim 1, wherein T is unsubstituted aryl.

10. A compound of claim 1, wherein T is unsubstituted phenyl, naphthyl, biphenyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl.

11. A compound of claim 10, wherein T is 2-naphthyl, biphen-4-yl, 1,2,3,4-tetrahydroquinolin-6-yl, or 1,2,3,4-tetrahydroquinolin-7-yl.

12. A compound of claim 1, wherein T is substituted aryl

13. A compound of claim 12, wherein T is substituted phenyl, naphthyl, biphenyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

14. A compound of claim 12, wherein T is phenyl substituted from 1 to 5 times with, C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁₋-C₆ alkyl), wherein each R¹⁶ is independently H or C₁-C₆ alkyl or a combination thereof.

15. A compound of claim 14, wherein T is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, or 4-acetylaminophenyl.

16. A compound of claim 1, wherein T is biphenyl substituted from 1 to 9 times with, C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

17. A compound of claim 1, wherein T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

18. A compound of claim 17, wherein T is 6-methoxy-2-naphthyl, 7-methoxy-2-naphthyl, 6-hydroxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxyethyl)-2-naphthyl, 7-(2-acetoxyethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, or 1-(2-acetoxyethyl)-3,4-dihydro-2H-quinolin-7-yl.

19. A compound of claim 1, wherein T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

20. A compound of claim 1, wherein T is unsubstituted heteroaryl.

21. A compound of claim 20, wherein T is quinolinyl, indolyl, isoquinolinyl, pyridyl, pyrimidinyl, pyrazinyl, or quinoxalinyl.

22. A compound of claim 21, wherein T is 2-quinolinyl, 6-quinolinyl, 7-quinolinyl, 6-isoquinolinyl, 2-pyridyl, 2-pyrimidinyl, 2-pyrazinyl, or 2-quinoxalinyl.

23. A compound of claim 1, wherein T is substituted heteroaryl.

24. A compound of claim 23, wherein T is substituted quinolinyl, indolyl, isoquinolinyl, pyridyl, pyrimidinyl, pyrazinyl, or quinoxalinyl.

25. A compound of claim 24, wherein T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

26. A compound of claim 24, wherein T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

27. A compound of claim 1, wherein T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

28. A compound of claim 27, wherein the N-substituent is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl.

29. A compound of claim 1, wherein Z is 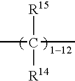
wherein 1 to 6 nonadjacent 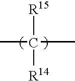
units are replaced with O.

30. A compound of claim 1, wherein R¹⁴ and R¹⁵ are hydrogen.

31. A compound of claim 1, wherein Z is
—(CH₂)₀₋₆—C(O)—NR¹⁶—(CH₂)₀₋₆— wherein 0 to 6 nonadjacent methylene units are replaced with O, NR¹⁶, S or a combination thereof; or
—(CH₂)₀₋₆—NR¹⁶—(C(O)—CH₂)₀₋₆— wherein 0 to 6 nonadjacent methylene units are replaced with O, NR¹⁶, S or a combination thereof; and
R¹⁶ is as defined in claim 1.

32. A compound of claim 29, wherein Z is —O—(CH₂)₂₋₃—O—(CH₂)₁₋₂—, —O—(CH₂)₃₋₄—O—, O—(CH₂)₁₋₂—, —(CH₂)—O—(CH₂)₂₋₃—O—(CH₂)₀₋₁—, —C(O)—NR¹⁶—(CH₂)₂—, —C(O)—NR¹⁶—(CH₂)₂—O—, or —O—(CH₂)₃—S—(CH₂)₁—.

33. A compound of claim 1, wherein when W is absent, Z is hydroxyl, C₁-C₁₂ alkyl wherein 1 to 6 nonadjacent methylene units are replaced with O, or —(CH₂)₀₋₆—C(O)—NR¹⁶—(CH₂)₀₋₅—CH₃ wherein 0 to 6 nonadjacent methylene units are replaced with O.

34. A compound of claim 1, wherein Z is —O—(CH₂)₃—O—(CH₂)—.

35. A compound of claim 1, wherein W is unsubstituted or substituted phenyl.

36. A compound of claim 1, wherein W is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, or 4-acetylaminophenyl.

37. A compound of claim 1, wherein W is 2-methoxyphenyl.

38. A compound of claim 1, wherein W is unsubstituted or substituted heteroaryl

39. A compound of claim 38, wherein W is unsubstituted indolyl.

40. A compound of claim 39, wherein W is 1H-Indol-3-yl.

41. A compound of claim 1, wherein Z is —O—(CH₂)₃—O—CH₂—, and W is 2-methoxyphenyl.

42. A compound of claim 1, wherein Q is —CH₂—O—, —CH₂—S— or —CH₂—O—CH₂— and T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

43. A compound of Formula II 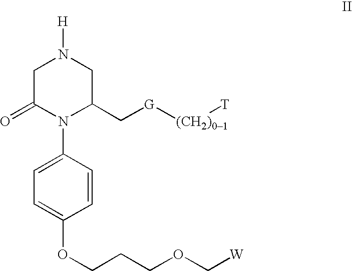
or a pharmaceutically acceptable salt thereof, wherein
G is O or S;
T is substituted or unsubstituted aryl, or substituted or unsubstituted heteroaryl; and
W is substituted or unsubstituted aryl, or substituted or unsubstituted heteroaryl.

44. A compound of claim 43, wherein T is substituted aryl.

45. A compound of claim 44, wherein T is substituted phenyl, naphthyl, biphenyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

46. A compound of claim 43, wherein T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

47. A compound of claim 43, wherein T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

48. A compound of claim 43, wherein T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

49. A compound of claim 43, wherein T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

50. A compound of claim 43, wherein T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

51. A compound of claim 50, wherein the N-substituent is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl.

52. A compound of claim 43, wherein W is unsubstituted or substituted phenyl.

53. A compound of claim 52, wherein W is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxyethyl)-phenyl, 3-(2-acetoxyethyl)-phenyl, 4-(2-acetoxyethyl)-phenyl, N,N-dimethyl-benzamide-4-yl, or 4-acetylaminophenyl.

54. A compound of claim 43, wherein W is 2-methoxyphenyl.

55. A compound of claim 43, wherein T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxyethyl)-3,4-dihydro-2H-quinolin-7-yl and W is 2-methoxyphenyl.

56. The compound
(6S)-1-{4-[3-(2-methoxybenzyloxy)-propoxy]-phenyl}-6-(naphthalen-2-yloxymethyl)-piperazin-2-one;
(6R)-6-(3,4-dichlorobenzyloxymethyl)-1-{4-[3-(2-methoxybenzyloxy)-propoxy]-phenyl}-piperazin-2-one;
(6R)-6-(2-fluorobenzyloxymethyl)-1-{4-[3-(2-methoxybenzyloxy)-propoxy]-phenyl}-piperazin-2-one;
[6-([2R]-1-{4-[3-(2-methoxybenzyloxy)-propoxy]-phenyl}-6-oxopiperazin-2-ylmethoxy)-3-oxo-2,3-dihydrobenzo[1,4]oxazin-4-yl]-acetic acid methyl ester;
propionic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester;
3-[6-([2R]-1-{4-[3-(2-methoxybenzyloxy)-propoxy]-phenyl}-6-oxopiperazin-2-ylmethoxy)-2,3-dihydroindol-1-yl]-propionic acid methyl ester; or
[5-([2S]-1-{4-[3-(2-methoxybenzyloxy)-propoxy]-phenyl}-6-oxopiperazin-2-ylmethoxy)-indol-1-yl]-acetic acid methyl ester.

57. A pharmaceutical composition comprising a compound of any of claims 1-56, admixed with a pharmaceutically acceptable carrier, diluent, or excipient.

58. A method of inhibiting renin in a mammal comprising administering to the mammal in need thereof an effective amount of a compound of any of claims 1-56.

59. A method of treating or preventing hypertension in a mammal comprising administering to the mammal in need thereof an effective amount of a compound of any of claims 1-56.

60. A method of treating or preventing congestive heart failure in a mammal comprising administering to the mammal in need thereof an effective amount of a compound of any of claims 1-56.

61. A method of treating or preventing stroke in a mammal comprising administering to the mammal in need thereof an effective amount of a compound of any of claims 1-56.

62. A method of treating or preventing myocardial infarction in a mammal comprising administering to the mammal in need thereof an effective amount of a compound of any of claims 1-56.
(6R)-6-(3,4-difluorobenzyloxymethyl)-1-{4-[3-(2-methoxybenzyloxy)-propoxy]-phenyl}-piperazin-2-one;
(6R)-6-(4-chlorobenzyloxymethyl)-1-{4-[3-(2-methoxybenzyloxy)-propoxy]-phenyl}-piperazin-2-one;
(6R)-6-(3-chlorobenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(4-methylbenzyloxymethyl)-piperazin-2-one;
(6R)-6-(4-fluorobenzyloxymethyl)-1-{4-[3-(2-methoxybenzyloxy)-propoxy]-phenyl}-piperazin-2-one;
(6R)-6-(3-methoxybenzyloxymethyl)-1-{4-[3-(2-methoxybenzyloxy)-propoxy]-phenyl}-piperazin-2-one;
(6R)-6-(2-methoxybenzyloxymethyl)-1-{4-[3-(2-methoxybenzyloxy)-propoxy]-phenyl}-piperazin-2-one;
(6R)-6-(3,5-difluorobenzyloxymethyl)-1-{4-[3-(2-methoxybenzyloxy)-propoxy]-phenyl}-piperazin-2-one;
(6R)-6-(4-methoxybenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(4-trifluoromethylbenzyloxymethyl)-piperazin-2-one;
(6R)-6-(2-chlorobenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(3-methylbenzyloxymethyl)-piperazin-2-one;
(6R)-6-(2,6-difluorobenzyloxymethyl)-1-{4-[3-(2-methoxybenzyloxy)-propoxy]-phenyl}-piperazin-2-one;
(6R)-6-(2,6-dichlorobenzyloxymethyl)-1-{4-[3-(2-methoxybenzyloxy)-propoxy]-phenyl}-piperazin-2-one;
(6R)-6-(3-fluorobenzyloxymethyl)-1-{4-[3-(2-methoxybenzyloxy)-propoxy]-phenyl}-piperazin-2-one;
(6R)-6-(4-fluoro-2-trifluoromethylbenzyloxymethyl)-1-{4-[3-(2-methoxybenzyloxy)-propoxy]-phenyl}-piperazin-2-one;
(6R)-6-(3,5-dichlorobenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(2-methylbenzyloxymethyl)-piperazin-2-one;
(6R)-6-(2-chloro-4-fluorobenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(pyridin-3-ylmethoxymethyl)-piperazin-2-one;
(6R)-6-(4-chloro-3-trifluoromethylbenzyloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(pyridin-4-ylmethoxymethyl)-piperazin-2-one;
(6R)-6-(4-fluoro-3-trifluoromethylbenzyloxymethyl)-1-{4-[3-(2-methoxybenzyloxy)-propoxy]-phenyl}-piperazin-2-one;
(6R)-6-(4-fluoro-3-methylbenzyloxymethyl)-1-{4-[3-(2-methoxybenzyloxy)-propoxy]-phenyl}-piperazin-2-one;
(6R)-4-(1-{4-[3-(2-methoxybenzyloxy)-propoxy]-phenyl}-6-oxopiperazin-2-ylmethoxymethyl)-benzonitrile;
(6R)-1-{4-[3-(2-methoxybenzyloxy)-propoxy]-phenyl}-6-(pyridin-2-ylmethoxymethyl)-piperazin-2-one;
(6R)-6-(4-bromobenzyloxymethyl)-1-{4-[3-(2-methoxybenzyloxy)-propoxy]-phenyl}-piperazin-2-one;
(2R)-1-{4-[3-(2-methoxybenzyloxy)-propoxy]-phenyl}-2-(naphthalen-2-yloxymethyl)-piperazine;
(2R)-2-(4-methoxybenzyloxymethyl)-1-{4-[3-(2-methoxy-benzyloxy)-propoxy]-phenyl}-piperazine;
(2R)-1-[4-(3-benzyloxypropoxy)-phenyl]-2-(4-methoxybenzyloxymethyl)-piperazine;
(2R)-1-(4-benzyloxyphenyl)-2-(naphthalen-2-yloxymethyl)-piperazine;
(2R)-1-(4-benzyloxyphenyl)-2-(4-methoxybenzyloxymethyl)-piperazine;
(6R)-1-{4-[3-(2-methoxybenzyloxy)-propoxy]-phenyl}-6-(naphthalen-2-yloxymethyl)-piperazin-2-one;
(2R)-1-[4-(3-benzyloxypropoxy)-phenyl]-2-(naphthalen-2-yloxymethyl)-piperazine;
(6R)-1-{3-fluoro-4-[3-(2-methoxybenzyloxy)-propoxy]-phenyl}-6-(naphthalen-2-yloxymethyl)-piperazin-2-one;
(2R)-1-{4-[3-(2-methoxybenzyloxy)-propoxy]-phenyl}-2-(5,6,7,8-tetrahydronaphthalen-2-yloxymethyl)-piperazine;
(6R)-1-{4-[3-(2-methoxybenzyloxy)-propoxy]-phenyl}-6-(quinolin-7-yloxymethyl)-piperazin-2-one;
(6R)-1-{4-[3-(2-methoxybenzyloxy)-propoxy]-phenyl}-6-(1,2,3,4-tetrahydroquinolin-7-yloxymethyl)-piperazin-2-one;
(6R)-1-{3,5-difluoro-4-[3-(2-methoxybenzyloxy)-propoxy]-phenyl}-6-(naphthalen-2-yloxymethyl)-piperazin-2-one;
(6R)-6-[1-(3-hydroxypropyl)-1,2,3,4-tetrahydroquinolin-7-yloxymethyl]-1-{4-[3-(2-methoxybenzyloxy)-propoxy]-phenyl}-piperazin-2-one;
(6R)-6-benzyloxymethyl-1-{4-[3-(2-methoxybenzyloxy)-propoxy]-phenyl}-piperazin-2-one;
(6S)-6-(4-fluorobenzyloxymethyl)-1-{4-[3-(2-methoxybenzyloxy)-propoxy]-phenyl}-piperazin-2-one; 4-[(2R)-2-(naphthalen-2-yloxymethyl)-6-oxopiperazin-1-yl]-N-phenethylbenzamide;
(6R)-1-[4-methoxy-3-(3-methoxypropoxy)-phenyl]-6-(naphthalen-2-yloxymethyl)-piperazin-2-one;
N-(2-ethoxyethyl)-4-[(2R)-2-(naphthalen-2-yloxymethyl)-6-oxopiperazin-1-yl]-benzamide;
N-[2-(3-methoxyphenyl)-ethyl]-4-[(2R)-2-(naphthalen-2-yloxymethyl)-6-oxopiperazin-1-yl]-benzamide;
(6R)-6-(isoquinolin-7-yloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(quinolin-6-yloxymethyl)-piperazin-2-one;
4-[(2R)-2-(naphthalen-2-yloxymethyl)-6-oxopiperazin-1-yl]-N-(2-phenoxyethyl)-benzamide;
(6R)-6-(1-acetyl-1,2,3,4-tetrahydroquinolin-6-yloxymethyl)-1-{4-[3-(2-methoxybenzyloxy)-propoxy]-phenyl}-piperazin-2-one;
(6R)-1-{4-[3-(2-methoxybenzyloxy)-propoxy]-phenyl}-6-(1-thiazol-4-ylmethyl-1,2,3,4-tetrahydroquinolin-7-yloxymethyl)-piperazin-2-one;
2-[7-(1-{4-[3-(2-methoxybenzyloxy)-propoxy]-phenyl}-6-oxopiperazin-2R-ylmethoxy)-3,4-dihydro-2H-quinolin-1-yl]-acetamide;
(6R)-6-[1-(2-hydroxyethyl)-1,2,3,4-tetrahydroquinolin-7-yloxymethyl]-1-{4-[3-(2-methoxybenzyloxy)-propoxy]-phenyl}-piperazin-2-one;
naphthalene-2-carboxylic acid (2R)-1-{4-[3-(2-methoxy-benzyloxy)-propoxy]-phenyl}-6-oxo-piperazin-2-yl methyl ester;
4-methyl-benzoic acid (2R)-1-{4-[3-(2-methoxy-benzyloxy)-propoxy]-phenyl}-6-oxo-piperazin-2-yl methyl ester;
4-chloro-benzoic acid (2R)-1-{4-[3-(2-methoxy-benzyloxy)-propoxy]-phenyl}-6-oxo-piperazin-2-yl methyl ester;
benzoic acid (2R)-1-{4-[3-(2-methoxybenzyloxy)-propoxy]-phenyl}-6-oxopiperazin-(2R)-yl methyl ester;
(2R)-1-{4-[3-(4-chlorobenzyloxy)-propoxy]-phenyl}-2-(4-methoxybenzyloxymethyl)-piperazine;
(2R)-1-{4-[3-(3,4-dichlorobenzyloxy)-propoxy]-phenyl}-2-(4-methoxybenzyloxymethyl)-piperazine;
(2R)-1-{4-[3-(3-chlorobenzyloxy)-propoxy]-phenyl}-2-(4-methoxybenzyloxymethyl)-piperazine;
(2R)-2-(4-methoxybenzyloxymethyl)-1-{4-[3-(4-methoxybenzyloxy)propoxy]-phenyl}-piperazine;
(2R)-1-{4-[3-(2-chlorobenzyloxy)-propoxy]-phenyl}-2-(4-methoxybenzyloxymethyl)-piperazine;
(2R)-1-{4-[3-(3,5-difluorobenzyloxy)-propoxy]-phenyl}-2-(4-methoxybenzyloxymethyl)-piperazine;
(2R)-2-(4-methoxybenzyloxymethyl)-1-{4-[3-(4-methylbenzyloxy)propoxy]-phenyl}-piperazine;
(2R)-2-(4-methoxybenzyloxymethyl)-1-{4-[3-(3-methoxybenzyloxy)-propoxy]-phenyl}-piperazine;
(2R)-2-(4-methoxybenzyloxymethyl)-1-{4-[3-(2-methoxyphenoxy)-propoxymethyl]-phenyl}-piperazine;
(6R)-6-(4-fluorobenzyloxymethyl)-1-{4-[4-(2-methoxyphenoxy)-butoxy]-phenyl}-piperazin-2-one;
(2R)-1-{4-[2-(2-methoxybenzyloxy)-ethoxymethyl]-phenyl}-2-(4-methoxybenzyloxymethyl)-piperazine;
(6R)-1-{4-[4-(2-methoxyphenoxy)-butoxy]-phenyl}-6-(naphthalen-2-yloxymethyl)-piperazin-2-one;
(6R)-6-(4-fluorobenzyloxymethyl)-1-{4-[2-(2-methoxybenzyloxy)-ethoxymethyl]-phenyl}-piperazin-2-one;
(2R)-2-(4-methoxybenzyloxymethyl)-1-{4-[4-(2-methoxyphenoxy)-butoxy]-phenyl}-piperazine;
(6R)-6-(4-fluorobenzyloxymethyl)-1-{4-[3-(2-methoxyphenoxy)-propoxymethyl]-phenyl}-piperazin-2-one;
(6R)-1-{4-[3-(2-methoxybenzyloxy)-propoxy]-phenyl}-6-(quinolin-2-yloxymethyl)-piperazin-2-one;
(6R)-1-{4-[3-(2-methoxybenzyloxy)-propoxy]-phenyl}-6-(quinoxalin-2-yloxymethyl)-piperazin-2-one;
(6R)-1-{4-[3-(2-methoxybenzyloxy)-propoxy]-phenyl}-6-(pyrazin-2-yloxymethyl)-piperazin-2-one;
(6R)-1-{4-[3-(2-methoxybenzyloxy)-propoxy]-phenyl}-6-(pyridin-2-yloxymethyl)-piperazin-2-one;
(6R)-1-{4-[3-(2-methoxybenzyloxy)-propoxy]-phenyl}-6-(pyrimidin-2-yloxymethyl)-piperazin-2-one;
2-methoxy-N-([2R]-1-{4-[3-(2-methoxybenzyloxy)-propoxy]-phenyl}-6-oxo-piperazin-2-ylmethyl)-benzamide;
4-chloro-N-([2R]-1-{4-[3-(2-methoxy-benzyloxy)-propoxy]-phenyl}-6-oxo-piperazin-2-ylmethyl)-benzamide;
N-([2R]-1-{4-[3-(2-methoxy-benzyloxy)-propoxy]-phenyl}-6-oxo-piperazin-2-ylmethyl)-benzamide;
naphthalene-2-carboxylic acid ([2R]-1-{4-[3-(2-methoxy-benzyloxy)-propoxy]-phenyl}-6-oxo-piperazin-2-ylmethyl)-amide;
2-fluoro-N-([2R]-1-{4-[3-(2-methoxy-benzyloxy)-propoxy]-phenyl}-6-oxo-piperazin-2-ylmethyl)-benzamide;
(2R)-1-{4-[3-(2-fluorobenzyloxy)-propoxy]-phenyl}-2-(naphthalen-2-yloxymethyl)-piperazine;
(2R)-1-{4-[3-(2-ethoxybenzyloxy)-propoxy]-phenyl}-2-(naphthalen-2-yloxymethyl)-piperazine;
(2R)-1-{4-[3-(3-methoxybenzyloxy)-propoxy]-phenyl}-2-(naphthalen-2-yloxymethyl)-piperazine;
(2R)-1-{4-[3-(2-methoxybenzyloxy)-propoxy]-phenyl}-2-(naphthalen-2-ylmethoxymethyl)-piperazine;
(2R)-2-(biphenyl-3-ylmethoxymethyl)-1-{4-[3-(2-methoxybenzyloxy)-propoxy]-phenyl}-piperazine;
(6R)-6-(biphenyl-4-yloxymethyl)-1-(4-[3-(2-methoxybenzyloxy)-propoxy]-phenyl)-piperazin-2-one;
N-[4-([2R]-1-{4-[3-(2-methoxybenzyloxy)-propoxy]-phenyl}-6-oxopiperazin-2-ylmethoxy)-phenyl]-acetamide;
(2R)-1-{4-[2-(2-methoxybenzyloxy)-ethoxymethyl]-phenyl}-2-(naphthalene-2-yloxymethyl)-piperazine;
4-([2R]-1-{4-[3-(2-methoxy-benzyloxy)-propoxy]-phenyl}-6-oxo-piperazin-2-ylmethoxy)-N,N-dimethyl-benzamide;
2-[(5R)-3-(2-methoxybenzyloxy)-propoxy]-5-[2-(naphthalene-2-yloxymethyl)-6-oxopiperazin-1-yl]-benzoic acid methyl ester;
(2R)-1-(4-[3-(2-methoxyphenoxy)-propoxymethyl]-phenyl)-2-(naphthalene-2-yloxymethyl)-piperazine;
2-[(5R)-[3-(2-methoxybenzyloxy)-propoxy]-5-[2-(naphthalene-2-yloxymethyl)-6-oxopiperazin-1-yl]-benzoic acid;
(2R)-1-(4-methoxymethylphenyl)-2-(naphthalene-2-yloxymethyl)-piperazine;
(6R)-1-(3-chloro-4-[3-(2-methoxybenzyloxy)-propoxy]-phenyl)-6-(naphthalene-2-yloxymethyl)-piperazin-2-one;
(6R)-1-{4-[3-(2-methoxybenzylsulfanyl)-propoxy]-phenyl}-6-(naphthalene-2-yloxymethyl)-piperazin-2-one;
(2R)-1-{4-[3-(2-methoxybenzylsulfanyl)-propoxy]-phenyl}-2-(naphthalene-2-yloxymethyl)-piperazine;
(6R)-1-{4-[3-(2-methoxybenzyloxy)-propoxy]-3-methyl-phenyl}-6-(naphthalene-2-yloxymethyl)-piperazin-2-one;
(2R)-1-{4-{2-[2-(2-methoxyphenyl)-ethoxy]-ethoxy}-ethoxy}-phenyl)-2-(naphthalene-2-yloxymethyl)-piperazine;
(6R)-1-{4-[3-(2-methoxybenzyloxy)-propoxy]-phenyl}-6-(7-methoxynaphthalen-2-yloxymethyl)-piperazin-2-one;
(6R)-6-(biphenyl-3-yloxymethyl)-1-{4-[3-(2-methoxybenzyloxy)-propoxy]-phenyl}-piperazin-2-one;
(6R)-1-(3-methoxy-4-[3-(2-methoxybenzyloxy)-propoxy]-phenyl)-6-(naphthalene-2-yloxymethyl)-piperazin-2-one;
(2R)-1-{4-[4-(2-methoxyphenoxy)-butoxy]-phenyl}-2-(naphthalene-2-yloxymethyl)-piperazine;
[6-([2R]-1-{4-[3-(2-methoxybenzyloxy)-propoxy]-phenyl}-6-oxopiperazin-2-ylmethylsulfanyl)-3-oxo-2,3-dihydrobenzo[1,4]oxazin-4-yl]-acetic acid ethyl ester;
6-([2R]-1-{4-[3-(2-methoxybenzyloxy)-propoxy]-phenyl}-6-oxopiperazin-2-ylmethylsulfanyl)-4-(3-methoxypropyl)-4H-benzo[1,4]oxazin-3-one;
N-{2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl}-acetamide;
3-[7-([2R]-1-{4-[3-(2-methoxybenzyloxy)-propoxy]-phenyl}-6-oxopiperazin-2-ylmethoxy)-3,4-dihydro-2H-quinolin-1-yl]-propionic acid methyl ester;
acetic acid 2-[7-([2R]-1-{4-[3-(2-methoxybenzyloxy)-propoxy]-phenyl}-6-oxopiperazin-2-ylmethoxy)-3,4-dihydro-2H-quinolin-1-yl]-ethyl ester;
N-{2-[7-([2R]-1-{4-[3-(2-methoxybenzyloxy)-propoxy]-phenyl}-piperazin-2-ylmethoxy)-3,4-dihydro-2H-quinolin-1-yl]-ethyl}-acetamide;
3-[5-([2R]-1-{4-[3-(2-methoxybenzyloxy)-propoxy]-phenyl}-6-oxopiperazin-2-ylmethoxy)-indol-1-yl]-propionic acid methyl ester;
7-([2S]-1-{4-[3-(2-methoxybenzyloxy)-propoxy]-phenyl}-6-oxopiperazin-2-ylmethoxy)-1-(3-methoxypropyl)-3,4-dihydro-1H-quinolin-2-one;
(6R)-1-{4-[3-(2-methoxybenzyloxy)-propoxy]-phenyl}-6-[1-(3-methoxypropyl)-1,2,3,4-tetrahydroquinolin-7-yloxymethyl]-piperazin-2-one;
7-([2R]-1-{4-[3-(2-methoxybenzyloxy)-propoxy]-phenyl}-6-oxopiperazin-2-ylmethoxy)-1-(3-methoxypropyl)-3,4-dihydro-1H-quinolin-2-one;
[7-([2R]-1-{4-[3-(2-methoxybenzyloxy)-propoxy]-phenyl}-6-oxopiperazin-2-ylmethoxy)-2-oxo-3,4-dihydro-2H-quinolin-1-yl]-acetic acid methyl ester;
[7-([2R]-1-{4-[3-(2-methoxybenzyloxy)-propoxy]-phenyl}-6-oxopiperazin-2-ylmethoxy)-3,4-dihydro-2H-quinolin-1-yl]-acetic acid methyl ester;
N-{2-[5-([2R]-1-{4-[3-(2-methoxybenzyloxy)-propoxy]-phenyl}-6-oxopiperazin-2-ylmethoxy)-indol-1-yl]-ethyl}-acetamide;
N-{2-[7-([2R]-1-{4-[3-(2-fluorobenzyloxy)-propoxy]-phenyl}-6-oxopiperazin-2-ylmethoxy)-3,4-dihydro-2H-quinolin-1-yl]-ethyl}-acetamide;
[6-([2R]-1-{4-[3-(2-methoxybenzyloxy)-propoxy]-phenyl}-6-oxopiperazin-2-ylmethoxy)-indol-1-yl]-acetic acid ethyl ester;
[5-([2R]-1-{4-[3-(2-methoxybenzyloxy)-propoxy]-phenyl}-6-oxopiperazin-2-ylmethoxy)-2-methyl-indol-1-yl]-acetic acid methyl ester;

63. A method of treating or preventing glaucoma in a mammal comprising administering to the mammal in need thereof an effective amount of a compound of any of claims 1-56.

64. A method of providing end organ protection in a mammal comprising administering to the mammal in need thereof an effective amount of a compound of any of claims 1-56.

65. A method of treating or preventing hyperaldosteronism in a mammal comprising administering to the mammal in need thereof an effective amount of a compound of any of claims 1-56.

66. A process for preparing a compound of claim 1 comprising the steps of:
a) acylation of a protected para-hydroxy aniline 1, wherein P¹ is an amine protecting group, to afford the intermediate 2 where R²⁰ is halo and R² is as defined in claim 1; 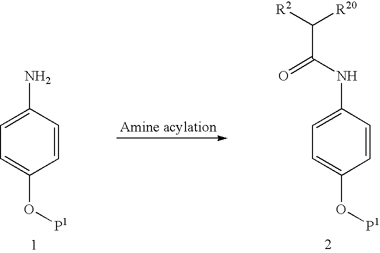
b) contacting 2 with a suitable amine to afford the intermediate 3, where P² is a suitable anime protecting group; 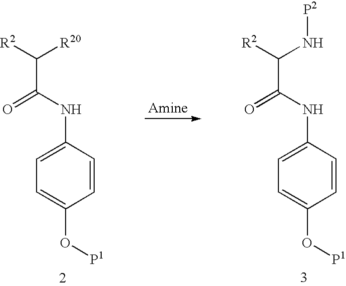
c) contacting 3 with a suitable epoxide to afford the intermediate 4, where R²¹ is halo and R¹ is as defined in claim 1; 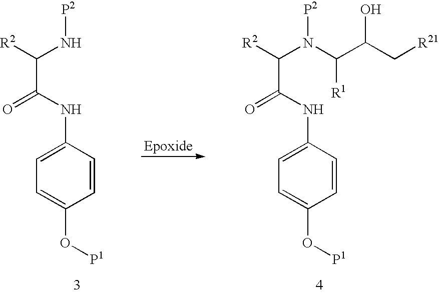
d) cyclization of 4 to afford 5; 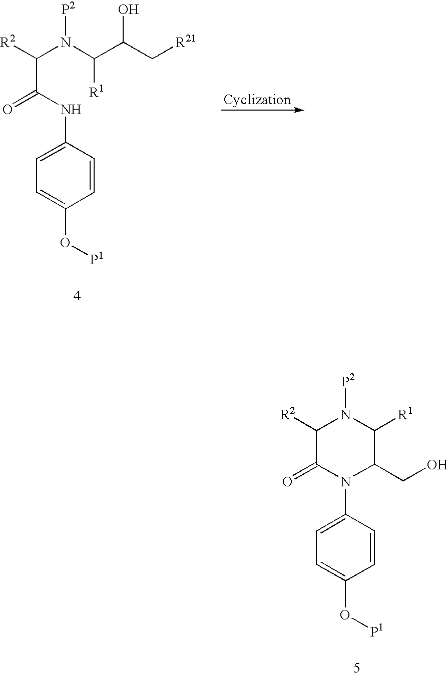
e) deprotection of 5 followed by protection of the piperazinone nitrogen with a suitable amine protecting group, P³, to afford 6; 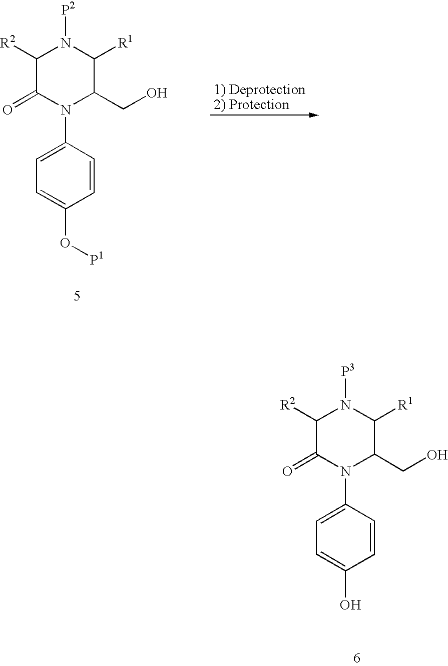
f) alkylation of 6 with a suitable alkylating agent to afford 7 where R²² along with the oxygen at the 4-position of the phenyl ring, is equivalent to -Z-W as is defined above in Formula I; 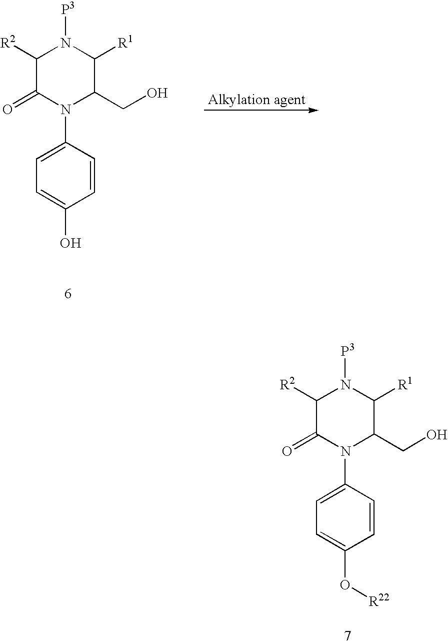
g) contacting 7 with an appropriate alcohol to afford 8, where R²³, along with the hydroxymethyl substituent of the piperazinone, is equivalent to -Q-T as is defined above in Formula I; 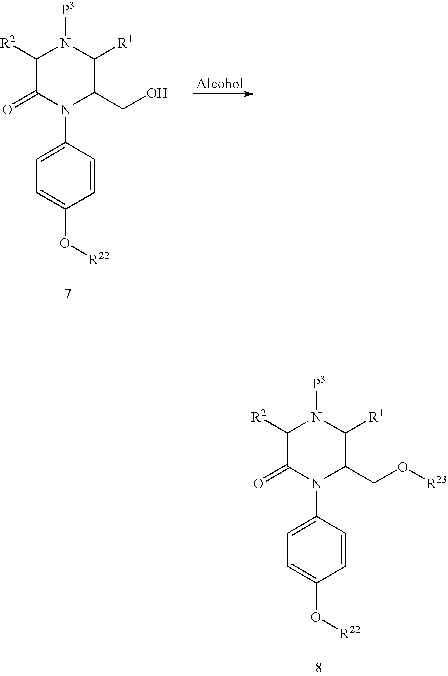
h) deprotection of 8 to afford 9

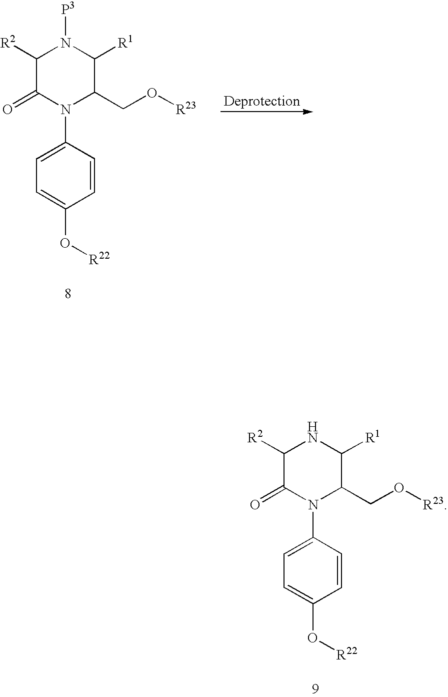

67. The process of claim 66 wherein 8 is reduced to the piperazine prior to deprotection.
